(19) United States
(12) Patent Application Publication
(10) Pub. No.: US 2022/0046948 A1
(43) Pub. Date: Feb. 17, 2022

(54) FORMULATIONS FOR EGG ORAL IMMUNOTHERAPY, METHODS OF MANUFACTURE, AND TREATMENTS FOR EGG ALLERGY

(71) Applicant: Aimmune Therapeutics, Inc., Brisbane, CA (US)

(72) Inventors: Reyna J. SIMON, Brisbane, CA (US); Nicholas William BIRRINGER, San Mateo, CA (US); Matthew SANDER, San Francisco, CA (US); Christina CAPULE, Moraga, CA (US); Kari Rose BROWN, Cary, NC (US); Stephen G. DILLY, San Mateo, CA (US); Daniel ADELMAN, Redwood City, CA (US)

(21) Appl. No.: 17/414,288

(22) PCT Filed: Dec. 17, 2019
(86) PCT No.: PCT/US2019/066922

§ 371 Date: Jun. 15, 2021

Related U.S. Application Data
(60) Provisional application No. 62/780,853, filed on Dec. 17, 2018.
(60) Provisional application No. 62/780,862, filed on Dec. 17, 2018.

(51) Int. Cl. A23J 1/08; A61K 35/57

(52) U.S. Cl. CPC A23J 1/08; A61K 35/57

(57) ABSTRACT

Described herein are egg white protein formulations, dosage containers containing the egg white protein formulations, methods of making the egg white protein formulations and dosage containers, and methods of controlling the quality of the egg white protein formulations, materials for manufacturing the egg white protein formulation (such as dried egg white protein powder), and the dosage containers. Further described herein are oral immunotherapy methods for treating an egg allergy. The method includes orally administering to the patient doses of a pharmaceutical composition comprising egg white protein according to an oral immunotherapy schedule. Also described herein are methods of adjusting a dosage of the pharmaceutical composition during oral immunotherapy if the patient experiences an adverse event related to the administration of a dose or a concurrent factor associated with increased sensitivity to an allergen that is not related to the administration of the dose.

### CROSS-REFERENCE TO RELATED APPLICATIONS

This application claims priority benefit of U.S. Provisional Application No. 62/780,853, filed Dec. 17, 2018; and U.S. Provisional Application No. 62/780,862, filed Dec. 17, 2018; the contents of each of which are incorporated by reference herein in their entirety.

### TECHNICAL FIELD

The present invention relates to formulations for egg oral immunotherapy and methods of manufacturing such formulations, and to oral immunotherapy treatments for egg allergy in a patient.

### BACKGROUND

IgE-mediated hen egg allergy is one of the most common food allergies of childhood and can be associated with severe immediate hypersensitivity reactions such as life-threatening anaphylaxis. The prevalence of egg allergy is estimated to be approximately 0.5% to 2.5% in western countries. While most children typically outgrow egg allergy over time, longitudinal studies have shown this may not occur until the second decade of life, with egg allergy persisting in approximately 20% of individuals aged 18 years.

Two main egg allergy phenotypes are recognized in the clinic: (1) allergy to both raw egg white and baked whole egg, and (2) allergy to raw egg white only. Approximately 20% to 30% of egg-allergic individuals are allergic to both raw egg white and baked whole egg, and 70% to 80% of egg-allergic individuals are allergic to raw egg white only.

The current standard of care for the management of egg allergy is dietary avoidance of egg and education of the patient/family on recognition and management of allergy symptoms and approximate use of rescue medications (e.g., epinephrine auto-injectors (EAIs)). However, avoidance of egg is exceptionally difficult for egg-allergic patients because of the ubiquity of egg as an ingredient in many food products, as well as its importance as a childhood nutrient. The burden of avoidance and constant fear of accidental exposure can negatively affect the health-related quality of life of individuals with egg allergy and their families. Compounding the medical need is that daily carriage and emergency use of EAIs for the treatment of anaphylaxis is thought to be inadequate, which can lead to adverse outcomes, including hospitalization and death.

It is generally believed that ingestion of just milligram quantities of egg protein can trigger allergic reactions, based on studies that have estimated population thresholds. While across the egg-allergic population the threshold levels at which allergic reactions are triggered varies widely, the ED10 (eliciting dose predicted to provoke a reaction in 10% of individuals with a specific food allergy) is estimated to range from 3.7 to 5.8 mg of egg protein. As a result, accidental allergic reactions to egg are common.

Currently available therapies are designed to treat only the symptoms of allergic reactions when they occur. Thus, in the absence of a cure, therapies with the potential to reduce the risk of severe allergic reaction in the event of an accidental exposure represent an urgent unmet medical need. However, the development of carefully calibrated dose forms for delivering egg allergens by oral immunotherapy has remained a challenge, particularly for lower-dose forms. For lower dose amounts, control of the allergenic material in an oral immunotherapy dose is particularly important to limit adverse events related to the therapy. Therefore, a bulk manufacturing process, particularly for when manufacturing low dose amounts, should have sufficient homogeneity throughout the mixed product do ensure adequate dose uniformity in the packaged product.

Oral immunotherapy (OIT) for egg allergy has been studied in recent years, which includes the oral administration of allergenic proteins to the patient in increasing doses to obtain a desensitized state. However, further improvement in the safety and/or efficacy of the treatment is desired.

### SUMMARY OF THE INVENTION

Described herein are egg white protein formulations, dosage forms containing egg white protein formulations, and methods of making such egg white protein formulations and dosage forms.

In some embodiments, a method of making an egg white protein formulation comprises: (a) mixing dried egg white protein powder with a first amount of a first diluent to form a first mixture; (b) passing the first mixture through a mesh screen; (c) mixing the first mixture with a second amount of the first diluent to form a second mixture after steps (a) and (b); (d) mixing the second mixture with a second diluent to form a third mixture; (e) mixing the third mixture at a higher shear force than used for mixing in step (c); and (f) mixing the third mixture with a lubricant to form the egg white protein formulation. In some embodiments, the egg white protein formulation has about 0.05 wt % to about 2.5 wt % of egg white protein. In some embodiments, the egg white protein formulation has about 0.1 wt % to about 0.7 wt % egg white protein. In some embodiments, step (b) comprises passing at least a portion of the second amount of the first diluent through the mesh screen with the first mixture. In some embodiments, step (b) comprises passing the second amount of the first diluent through the mesh screen with the first mixture. In some embodiments, step (c) comprises a plurality of sub-steps, wherein each sub-step comprises (i) adding a portion of the second amount of the first diluent to the first mixture, and (ii) mixing the portion of the second amount of the first diluent and the first mixture. In some embodiments, step (c) comprises three or more sub-steps. In some embodiments, step (c) comprises continuously mixing the first mixture and the second amount of the first diluent as the second amount of the first diluent is added to the first mixture. In some embodiments, the first mixture is mixed with the second amount of the first diluent in a tumble blender. In some embodiments, the second mixture is mixed with the second diluent in a tumble blender. In some embodiments, the third mixture is mixed using a conical mill. In some embodiments, the third mixture is mixed with the lubricant in a tumble blender. In some embodiments, a third amount of the first diluent is mixed with the third mixture. In some embodiments, the third amount of the first diluent and the lubricant are co-mixed with the third mixture. In some embodiments, step (f) comprises (i) passing the third mixture and the lubricant together through a mesh screen and (ii) mixing the third mixture and the lubricant to form the egg white protein formulation. In some embodiments, the egg white protein formulation is substantially free of colloidal silicon dioxide. In some embodiments, the egg white protein formulation is about 40 wt % to about 70 wt % of the first diluent. In some embodiments, the egg white protein formulation is about 30 wt % to about 50 wt % of the second diluent. In some embodiments, the egg white protein formulation is about 0.1 wt % to about 2 wt % of the lubricant.

In some embodiments, a method of making an egg white protein formulation comprises (a) mixing dried egg white protein powder with a first amount of a first diluent to form a first mixture; (b) mixing a second amount of the first diluent with the first mixture to form a second mixture; (c) mixing the second mixture with a second diluent to form a third mixture; (d) mixing the third mixture at a higher shear force than used for mixing in step (b); and (e) mixing the third mixture with a lubricant to form the egg white protein formulation. In some embodiments, the dried egg white protein powder is mixed with the first amount of the first diluent in step (a) at a higher shear force than used for mixing in step (b). In some embodiments, the egg white protein formulation has about 1 wt % to about 70 wt % egg white protein. In some embodiments, the dried egg white protein powder is mixed with the first amount of the first diluent in step (a) using a conical mill. In some embodiments, step (b) comprises two mixing sub-steps, wherein one mixing sub-step is at a higher shear force than the other mixing sub-step. In some embodiments, step (c) comprises mixing the second mixture with an additional amount of the first diluent prior to mixing with the second diluent to form the third mixture. In some embodiments, step (c) further comprises co-mixing the second mixture with an additional amount of the first diluent and the second diluent to form the third mixture. In some embodiments, the third mixture is mixed in step (d) using a conical mill. In some embodiments, the second amount of the first diluent and the first mixture are mixed in a tumble blender. In some embodiments, the second mixture is mixed with the second diluent in a tumble blender. In some embodiments, a third amount of the first diluent is mixed with the third mixture. In some embodiments, the third amount of the first diluent and the lubricant are co-mixed with the third mixture. In some embodiments, step (e) comprises (i) mixing a portion of the third mixture with a lubricant; (ii) passing the mixture of (i) through a mesh screen; and (iii) mixing the mixture of (ii) with an additional portion of the third mixture to form the egg white protein formulation. In some embodiments, the third mixture is mixed with the lubricant in a tumble blender. In some embodiments, the egg white protein formulation is about 9 wt % to about 85 wt % of the first diluent. In some embodiments, the egg white protein formulation is about 10 wt % to about 50 wt % of the second diluent. In some embodiments, the egg white protein formulation is about 10 wt % to about 20 wt % of the second diluent. In some embodiments, the egg white protein formulation is about 0.1 wt % to about 2 wt % of the lubricant. In some embodiments, the egg white protein formulation comprises a glidant. In some embodiments, the glidant is mixed with the egg white protein powder and the first amount of the first diluent during or prior to step (a). In some embodiments, the glidant is colloidal silicon dioxide. In some embodiments, the egg white protein formulation is about 2 wt % to about 70 wt % egg white protein. In some embodiments, the egg white protein formulation is substantially free of colloidal silicon dioxide, and may comprise about 1 wt % to about 5 wt % egg white protein.

In another embodiment, a method of making an egg white protein formulation comprises: (a) mixing dried egg white protein powder, a first diluent, and a glidant to form a first mixture; (b) mixing a second diluent and the first mixture at a higher shear force than used for mixing in step (a) to form a second mixture; (c) mixing the second mixture and a lubricant to form the egg white protein formulation. In some embodiments, the egg white protein formulation has about 50 wt % to about 80 wt % of egg white protein. In some embodiments, the dried egg white protein powder, the first diluent, and the glidant are mixed in a tumble blender. In some embodiments, the second diluent and the first mixture are mixed in step (b) using a conical mill. In some embodiments, the second mixture and the lubricant are mixed in a tumble blender. In some embodiments, a second amount of the first diluent is mixed with the second mixture. In some embodiments, the second amount of the first diluent and the lubricant are co-mixed with the second mixture. In some embodiments, the glidant comprises colloidal silicon dioxide. In some embodiments, step (c) comprises (i) mixing a portion of the second mixture and the lubricant; (ii) passing the portion of the second mixture and the lubricant through a mesh screen; and (iii) mixing the mixture of (ii) with an additional portion of the second mixture to form the egg white protein formulation.

In some embodiments of the above methods, the egg white protein formulation is made in a lot size of about 5 kg or more. In some embodiments, the egg white protein formulation is made in a lot size of about 5 kg to about 50 kg.

In some embodiments of the above methods, the method further comprises determining an egg white protein blend uniformity for the egg white protein formulation. In some embodiments, the egg white protein formulation has an egg white protein blend uniformity relative standard deviation (RSD) of about 15% or less.

In some embodiments of the above methods, the method further comprises packaging the egg white protein formulation in a plurality of dosage containers. In some embodiments, the dosage containers are capsules or sachets. In some embodiments, the method further comprises determining an egg white protein content uniformity for the plurality of dosage containers. In some embodiments, the plurality of dosage containers has an egg white protein content uniformity relative standard deviation (RDS) of about 15% or less.

In some embodiments of the above methods, the first diluent is pregelatinized starch.

In some embodiments of the above methods, the second diluent is microcrystalline cellulose.

In some embodiments of the above methods, the lubricant is magnesium stearate.

In some embodiments of the above methods, the dried egg white protein powder comprises about 50 wt % to about 90 wt % egg white protein.

In some embodiments of the above methods, formation of the dried egg white protein powder comprises spray drying liquid egg whites.

In some embodiments of the above methods, the egg white protein powder has had glucose removed.

In some embodiments of the above methods, the dried egg white powder has been pasteurized.

In some embodiments of the above methods, the dried egg white protein powder is derived from a chicken egg.

In some embodiments of the above methods, the method further comprises characterizing ovomucoid, ovalbumin, ovotransferrin, or lysozyme in the dried egg white protein powder.

In some embodiments of the above methods, the method further comprises characterizing ovomucoid, ovalbumin, ovotransferrin, or lysozyme in the egg white protein formulation.

In some embodiments, characterizing ovomucoid, ovalbumin, ovotransferrin, or lysozyme comprises obtaining a high-performance liquid chromatography (HPLC) profile. In some embodiments, the HPLC profile is a revere-phased HPLC (RP-HPLC) profile. In some embodiments, the HPLC profile is a size-exclusion chromatography HPLC (SEC-HPLC) profile. In some embodiments, the method comprises comparing the obtained HPLC profile to a reference HPLC profile.

In some embodiments, characterizing ovomucoid, ovalbumin, ovotransferrin, or lysozyme comprises quantifying an amount of ovomucoid, ovalbumin, ovotransferrin, or lysozyme. In some embodiments, quantifying the amount of ovomucoid, ovalbumin, ovotransferrin, or lysozyme comprises measuring an amount of ovomucoid, ovalbumin, ovotransferrin, or lysozyme compared to total protein in the egg white protein powder or the egg white protein formulation. In some embodiments, quantifying the amount of ovomucoid, ovalbumin, ovotransferrin, or lysozyme comprises measuring an amount of ovomucoid, ovalbumin, ovotransferrin, or lysozyme compared to a total amount of ovomucoid, ovalbumin, ovotransferrin, and lysozyme in the egg white protein powder or the egg white protein formulation.

In some embodiments, characterizing ovomucoid, ovalbumin, ovotransferrin, or lysozyme comprises measuring a potency of ovomucoid, ovalbumin, ovotransferrin, or lysozyme in the egg white protein powder or the egg white protein formulation. In some embodiments, the potency of ovomucoid, ovalbumin, ovotransferrin, or lysozyme in the egg white protein powder or the egg white protein formulation is measured relative to a potency of ovomucoid, ovalbumin, ovotransferrin, or lysozyme in a reference sample. In some embodiments, the potency of ovomucoid, ovalbumin, ovotransferrin, or lysozyme is measured using an immunoassay. In some embodiments, the immunoassay comprises the use of an antibody that specifically binds ovomucoid, ovalbumin, ovotransferrin, or lysozyme. In some embodiments, the immunoassay comprises the use of a pool of antibodies comprising two or more antibodies selected from the group consisting of an antibody that specifically binds ovomucoid, an antibody that specifically binds ovalbumin, an antibody that specifically binds ovotransferrin, or an antibody that specifically binds lysozyme. In some embodiments, the antibody is an IgE antibody or an IgG antibody. In some embodiments, the potency of ovomucoid, ovalbumin, ovotransferrin, or lysozyme is measured using an enzyme-linked immunosorbent assay (ELISA).

Further described herein is an egg white protein formulation made according to any one of the above methods.

In some embodiments, an egg white protein formulation comprises dried egg white protein powder, a first diluent, a second diluent, and a lubricant, wherein the egg white protein formulation is substantially free of colloidal silicon dioxide. In some embodiments, the egg white protein formulation comprises about 0.1 wt % to about 3.5 wt % egg white protein. In some embodiments, the first diluent is pregelatinized starch. In some embodiments, the second diluent is microcrystalline cellulose. In some embodiments, the lubricant is magnesium stearate. In some embodiments, the egg white protein formulation consists essentially of the egg white protein powder, the first diluent, the second diluent, and the lubricant.

Further described herein are methods of treating an egg allergy in a patient, and methods of adjusting a dosage of a pharmaceutical composition comprising egg white protein during oral immunotherapy for an egg allergy.

In one embodiment, a method of treating an egg allergy in a patient comprises orally administering to the patient a plurality of doses of a pharmaceutical composition comprising egg white protein according to an oral immunotherapy schedule comprising: (a) an up-dosing phase comprising orally administering to the patient a series of escalating doses of about 1 mg to about 300 mg of egg white protein, wherein a given dose is administered to the patient for at least two weeks before the dose is escalated, and wherein the up-dosing phase is about 20 weeks to about 44 weeks in length; and (b) a maintenance phase comprising orally administering to the patient a plurality of maintenance doses comprising egg white protein, wherein the maintenance phase is about 12 weeks in length or more.

In some embodiments, the patient has an egg-white-specific serum IgE (ew-IgE) level of about 7 kU_{A}/L or more at the start of treatment. In some embodiments, the patient has an egg-white-specific serum IgE (ew-IgE) level of about 5 kU_{A}/L or more at the start of treatment.

In some embodiments, the series of escalating doses administered to the patient during the up-dosing phase comprises at least a 1 mg dose of egg white protein and a 300 mg dose of egg white protein. In some embodiments, the series of escalating doses administered to the patient during the up-dosing phase comprises at least 10 different doses of egg white protein. In some embodiments, the series of escalating doses administered to the patient during the up-dosing phase comprises doses of about 1 mg, about 3 mg, about 6 mg, about 12 mg, about 20 mg, about 40 mg, about 80 mg, about 120 mg, about 160 mg, about 200 mg, about 240 mg, and about 300 mg of egg white protein.

In some embodiments, a dose administered during the up-dosing phase is escalated only if the patient tolerates the previous dose.

In some embodiments, the maximum dose administered to the patient during the up-dosing phase is about 300 mg of egg white protein.

In some embodiments, the maintenance dose administered to the patient during the maintenance phase is about 300 mg of egg white protein or more. In some embodiments, the maintenance dose administered to the patient during the maintenance phase is about 300 mg of egg white protein.

In some embodiments, the maintenance dose is administered to the patient only if the patient tolerates the maximum dose administered to the patient during the up-dosing phase.

In some embodiments, the patient tolerates a dose of about 600 mg raw egg white protein at the end of the maintenance phase. In some embodiments, the patient tolerates a dose of about 1000 mg raw egg white protein at the end of the maintenance phase. In some embodiments, the patient tolerates a dose of about 2000 mg raw egg white protein at the end of the maintenance phase. In some embodiments, the patient tolerates a cumulative dose of about 2000 mg cooked egg white protein at the end of the maintenance phase. In some embodiments, the patient tolerates a cumulative dose of about 2000 mg baked egg white protein at the end of the maintenance phase.

In some embodiments, the patient is unable to tolerate a dose of about 300 mg of raw egg white protein prior to the start of treatment. In some embodiments, the patient is unable to tolerate a cumulative dose of about 2000 mg of cooked egg white protein prior to the start of treatment. In some embodiments, the patient tolerates a cumulative dose of about 2000 mg of baked egg white protein prior to the start of treatment. In some embodiments, the patient is unable to tolerate a cumulative dose of about 2000 mg of baked egg white protein prior to the start of treatment. In some embodiments, the patient tolerates a cumulative dose of about 2000 mg of baked egg white protein prior to the start of treatment.

In some embodiments, the oral immunotherapy schedule comprises an initial escalation phase prior to the up-dosing phase, the initial escalation phase comprising orally administering to the patient a series of escalating doses of about 0.2 mg to about 2 mg of egg white protein in a single day, wherein a single administration of any given dose is administered to the patient, and wherein the doses are spaced by at least 15 minutes.

In some embodiments, the patient is treated according to the oral immunotherapy schedule only if the patient tolerates a dose of about 1.2 mg of raw egg white protein on the first day of treatment.

In some embodiments, the patient is about 4 years of age or older prior to the start of treatment. In some embodiments, the patient is about 4 years to about 26 years of age prior to the start of treatment.

Also described herein is a method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising: orally administering to the patient a first dose of the pharmaceutical composition; and orally administering to the patient a second dose of the pharmaceutical composition, wherein the second dose is reduced, skipped, or at least a portion of the dose is delayed if the patient experiences an adverse event related to the administration of the first dose. In some embodiments, the second dose is divided into a first portion and a second portion, wherein the first portion is administered according to a predetermined dosing schedule, and wherein the second portion is delayed relative to the predetermined dosing schedule, if the patient experiences the adverse event related to the administration of the first dose. In some embodiments, the second portion is delayed by about 8 hours to about 12 hours after the first portion is administered. In some embodiments, the second dose is skipped if the patient experiences the adverse event related to the administration of the first dose. In some embodiments, the second dose is reduced relative to the first dose if the patient experiences the adverse event related to the administration of the first dose. In some embodiments, subsequent doses of the pharmaceutical composition are reduced relative to the first dose for about one week or more prior to escalating the subsequent doses. In some embodiments, subsequent doses of the pharmaceutical composition are reduced relative to the first dose for about one week to about two weeks prior to attempting to escalate the subsequent doses. In some embodiments, subsequent doses of the pharmaceutical composition are reduced relative to the first dose for about one week to about two weeks prior to escalating the subsequent doses. In some embodiments, the adverse event related to the administration of the first dose is a mild allergenic adverse event. In some embodiments, the adverse event related to the administration of the first dose is a moderate allergenic adverse event or severe allergenic adverse event. In some embodiments, the first dose and the second dose are administered to the patient during the up-dosing phase of the oral immunotherapy. In some embodiments, the first dose and the second dose are administered to the patient during the maintenance phase of the oral immunotherapy.

Also described herein is a method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising: orally administering to the patient a first dose of the pharmaceutical composition; and orally administering to the patient a second dose of the pharmaceutical composition, wherein the second dose is reduced or skipped if the patient experiences a concurrent factor associated with increased sensitivity to an allergen that is not related to the administration of the first dose. In some embodiments, the concurrent factor associated with increased sensitivity to an allergen is an atopic disease flare-up, inflammation, an illness, or menses. In some embodiments, the second dose is skipped if the patient experiences the concurrent factor associated with increased sensitivity to an allergen not related to the administration of the first dose. In some embodiments, the second dose is reduced relative to the first dose if the patient experiences the concurrent factor associated with increased sensitivity to an allergen not related to the administration of the first dose. In some embodiments, subsequent doses of the pharmaceutical composition are reduced relative to the first dose for about one week or more prior to escalating the subsequent doses. In some embodiments, subsequent doses of the pharmaceutical composition are reduced relative to the first dose for about one week to about two weeks prior to attempting to escalate the subsequent doses. In some embodiments, subsequent doses of the pharmaceutical composition are reduced relative to the first dose for about one week to about two weeks prior to escalating the subsequent doses. In some embodiments, the first dose and the second dose are administered to the patient during the up-dosing phase of the oral immunotherapy. In some embodiments, the first dose and the second dose are administered to the patient during the maintenance phase of the oral immunotherapy. In some embodiments, the concurrent factor associated with increased sensitivity to an allergen is an unintended exposure to a food that the patient is allergic to.

In some embodiments of the above methods, the egg white protein in the pharmaceutical composition is raw egg white protein.

In some embodiments of the above methods, the pharmaceutical composition is mixed with a food vehicle prior to administration.

Further provided herein is a pharmaceutical composition for use in the methods of treating an egg allergy described above, wherein the pharmaceutical composition comprises the egg white protein formulation prepared according to the preceding methods of making an egg white protein formulation.

Further provided herein is a pharmaceutical composition for use in the manufacture of a medicament for a method of treating an egg allergy in a patient according to the methods of treating an egg allergy described above, wherein the pharmaceutical composition comprises the egg white protein formulation prepared according to the preceding methods of making an egg white protein formulation.

### BRIEF DESCRIPTION OF THE DRAWINGS

FIG. 1 shows an exemplary method of making egg white protein formulation, particularly for lower dose levels.

FIG. 2 shows an exemplary method of making egg white protein formulation, particularly for medium dose levels.

FIG. 3 shows an exemplary method of making egg white protein formulation, particularly for higher dose levels.

FIG. 4 shows an exemplary decision tree for adjusting a dosage depending on the severity of an adverse event related to the administration of the egg white protein during oral immunotherapy.

FIG. 5 shows a size-exclusion chromatography HPLC chromatogram of dried egg white protein powder, with ovalbumin, ovomucoid, ovotransferrin, and lysozyme identified.

FIG. 6 shows a reverse-phased HPLC chromatogram of dried egg white protein powder, with ovalbumin, ovomucoid, ovotransferrin, and lysozyme identified.

FIG. 7 shows an SDS-PAGE gel of dried egg white protein powder (lanes 3-5; about 2 μg protein per well) and protein standards (ovomucoid, ovalbumin, lysozyme, and ovotransferrin; lanes 7-10).

FIG. 8 shows an immunoblot of dried egg white protein powder (lanes 3-5) and protein standards (ovomucoid, ovalbumin, lysozyme, and ovotransferrin; lanes 7-10), which was stained to identify ovalbumin, ovomucoid, ovotransferrin, and lysozyme.

FIG. 9 shows a schematic of a clinical study of the pharmaceutical composition containing egg white protein.

FIG. 10 shows a schematic of a clinical study of the pharmaceutical composition containing egg white protein.

FIG. 11 shows a reverse-phased HPLC chromatogram of dried egg white protein powder using an improved RP-HPLC method, with the relative retention time peaks of ovomucoid, lysozyme, ovotransferrin, and ovalbumin identified.

FIG. 12A shows an exemplary method of making egg white protein formulation, particularly for lower dose levels.

FIG. 12B shows another exemplary method of making egg white protein formulation, particularly for lower dose levels.

FIG. 12C shows another exemplary method of making egg white protein formulation, particularly for lower dose levels.

FIG. 13A shows an exemplary method of making egg white protein formulation, particularly for medium dose levels.

FIG. 13B shows another exemplary method of making egg white protein formulation, particularly for medium dose levels.

FIG. 13C shows another exemplary method of making egg white protein formulation, particularly for medium dose levels.

FIG. 14A shows an exemplary method of making egg white protein formulation, particularly for higher dose levels.

FIG. 14B shows another exemplary method of making egg white protein formulation, particularly for higher dose levels.

### DETAILED DESCRIPTION

Described herein are egg white protein formulations, and methods of making such egg white protein formulations. The egg white protein formulations include dried egg white protein powder, along with one or more diluents, a lubricant, and (in some formulations) a glidant, and can be useful in treating egg allergy through an oral immunotherapy. Because highly allergic patients can be susceptible to small variations in the amount of allergen contained in the formulation, it is desirable to carefully calibrate the amount and quality of egg allergens contained within the formulations, for example by using the quality control and/or manufacturing processes described herein. The manufacturing methods described herein allow for the production of egg white protein formulations with carefully controlled amounts of egg allergens.

A significant challenge in manufacturing egg white protein formulations is the scale-up of lot size. Homogeneity in smaller, research bench top lots is more easily obtainable than large-scale manufacture for commercial production. Obtaining sufficiently homogenous formulation blends is particularly challenging, and important, for low-dose formulations, where patients consuming such dosages may be particularly sensitive to egg white allergens. The manufacturing methods described herein provide for scale-up of egg white protein formulation manufacture, including batches larger than 5 kilograms.

In some methods of making an egg white protein formulation, the method includes (a) mixing dried egg white protein powder with a first amount of a first diluent to form a first mixture; (b) passing the first mixture through a mesh screen; (c) mixing the first mixture with a second amount of the first diluent to form a second mixture after steps (a) and (b); (d) mixing the second mixture with a second diluent to form a third mixture; (e) mixing the third mixture at a higher shear force than used for mixing in step (c); and (f) mixing the third mixture with a lubricant to form the egg white protein formulation. In step (e), the third mixture is mixed to dissociate particle agglomerates. Step (c) may occur in a plurality of sub-steps to serially dilute the mixture with two or more different portions of the second amount of the first diluent. This method of manufacturing the formulation is particularly useful for formulations used to manufacture lower-dose dosage containers containing the formulation, such as doses of about 0.1 mg to about 12 mg. The formulation may have, for example, about 0.05 wt % to about 2.5 wt % of egg white protein. A glidant (e.g., colloidal silicon dioxide) need not be included in the lower-dose formulations.

In some methods of making an egg white protein formulation, the method includes (a) mixing dried egg white protein powder with a first amount of a first diluent to form a first mixture; (b) mixing a second amount of the first diluent with the first mixture to form a second mixture; (c) mixing the second mixture with a second diluent to form a third mixture; (d) mixing the third mixture at a higher shear force than used for mixing in step (b); and (e) mixing the third mixture with a lubricant to form the egg white protein formulation. In step (d), the third mixture is mixed to dissociate particle agglomerates in the mixture. Step (a) may also be performed at a higher shear force than used for mixing in step (b), which can dissociate particle agglomerates in the mixture. This method of manufacturing the formulation is particularly useful for formulations used to manufacture mid-dose dosage containers containing the formulation, such as doses of about 3 mg to about 240 mg. The formulation may have, for example, about 1 wt % to about 70 wt % of egg white protein.

In some methods of making an egg white protein formulation, the method includes (a) mixing dried egg white protein powder, a first diluent, and a glidant to form a first mixture; (b) mixing a second diluent and the first mixture at a higher shear force than used for mixing in step (a) to form a second mixture; (c) mixing the second mixture and a lubricant to form the egg white protein formulation. The mixing in step (b) is performed such that the mixing dissociates particle agglomerates. This method of manufacturing the formulation is particularly useful for formulations used to manufacture higher-dose dosage containers containing the formulation, such as doses of about 200 mg and larger (such as up to 600 mg, or up to 300 mg). The formulation may have, for example, about 50 wt % to about 80 wt % of egg white protein. The egg white protein formulations described herein comprise, consist essentially of, or consists of dried egg white protein powder, one or more diluents (e.g., two diluents), and a lubricant. In some embodiments, the egg white protein formulation is substantially free of a glidant or substantially free of colloidal silicon dioxide. Optionally, the egg white protein formulation comprises, consists essentially of, or consist of dried egg white protein powder, one or more diluents (e.g., two diluents), a lubricant, and a glidant (such as colloidal silicon dioxide).

The egg white protein formulation can be packaged in a dosage container, such as a capsule or a sachet. The dose of egg white protein in the dosage container depends on the amount of formulation packaged in the container and the concentration of egg white protein in the formulation. For example, the dose of egg white protein in the dosage container can be 0.1 mg to about 1000 mg of egg white protein.

The dried egg white protein powder used to manufacture the formulations, the manufactured egg white protein formulations, and the manufactured dosage containers containing the egg white protein formulations should be carefully controlled to ensure accurate administration of the egg white protein dose to a patient being treated for an egg allergy by oral immunotherapy. As further described herein, one or more methods can be used to assess the quality of the dried egg white protein powder, the egg white protein formulation, or the dosage containers. Such methods include measuring a protein content of the powder or formulation, characterizing one or more allergenic egg white proteins (e.g., ovalbumin, ovomucoid, ovotransferrin, and/or lysozyme) in the powder or formulation, determining a blend uniformity of a formulation, measuring a deliverable amount of formulation from a lot of dosage containers, or measuring content uniformity in a lot of dosage containers.

Further described herein is a method of treating an egg allergy in a patient, which includes orally administering to the patient a plurality of doses of a pharmaceutical composition comprising egg white protein according to an oral immunotherapy schedule comprising (a) an up-dosing phase comprising orally administering to the patient a series of escalating doses of about 1 mg to about 300 mg of egg white protein, wherein a given dose is administered to the patient for at least two weeks before the dose is escalated, and wherein the up-dosing phase is about 20 weeks to about 44 weeks in length; and (b) a maintenance phase comprising orally administering to the patient a plurality of maintenance doses comprising egg white protein, wherein the maintenance phase is about 12 weeks in length or more.

The dosage of the pharmaceutical composition administered to the patient may be adjusted (for example, by reducing a dose, skipping as dose, delaying (all or a portion) a dose administration, or delaying an increase of a dose) in response to an adverse event related to administration of the pharmaceutical composition, or some other concurrent factor associated with increased sensitivity to an allergen (such as an atopic disease flare-up, inflammation, an illness, menses, or unintended exposure to a food that the patient is allergic to) not related to administration of the pharmaceutical composition to the patient.

The oral immunotherapy (OIT) treatment schedule described herein is designed to safely desensitize a patient allergic to egg proteins so that the patient is able to ingest egg proteins through accidental exposure without a moderate allergenic adverse event or severe allergenic adverse event. The patient may maintain a cooked egg-avoidance diet after treatment, depending on the treatment outcome of the individual patient.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

An “adverse event” is any unfavorable and unintended sign (including an abnormal laboratory finding), symptom, or disease, or worsening thereof, temporally associated with the oral immunotherapy, whether or not related to the oral immunotherapy.

A composition “consisting essentially of” listed materials includes only those materials for at least 99.95 wt % of the composition. Additives, impurities, or other components may make up less than 0.05 wt % of the composition.

The term “cooked” refers to a heat treatment of a composition without burning the composition which results in at least partial modification or denaturation of one or more proteins in the composition. The term “baked” refers to heat treatment of a composition at a temperature of at least 176° C. for at least 20 minutes and without burning of the composition.

The term “desensitized” is used herein to refer to an increased allergic reaction threshold to a food allergen by a subject as a result of an oral immunotherapy for the food allergen. Desensitization to a food allergen can be tested using methods known in the art, including an oral food challenge. Desensitization may be partial, wherein the subject tolerates an increased amount of the food allergen compared to prior to treatment, but still reacts to higher doses of the food allergen; or the desensitization may be complete, wherein the patient tolerates all tested doses of the food allergen.

The terms “effective,” “efficacy,” or “effectiveness” are used herein to refer to the ability of a therapy to induce immune modulation, such as desensitization, or sustain a desired immune state, such as a desensitized state, unless otherwise indicated.

An “escalated dose” is the first dose administered to a patient that is higher than an immediately previous dose administered to the patient during the course of the oral immunotherapy.

As used herein, “maintenance phase” refers to a phase of an egg protein oral immunotherapy that includes administration of egg white protein (i.e., a maintenance dose) to the patient, and occurs after completion of an up-dosing phase.

As used herein, a “mild allergic adverse event” refers to an observed or experienced OIT-treatment-related allergic adverse event associated with transient discomfort, but does not require immediate medical intervention such as hospitalization or epinephrine, and does not substantially interfere with daily activities.

As used herein, a “moderate allergic adverse event” refers to an observed or experienced OIT-treatment-related allergic adverse event that is associated with discomfort of a sufficient degree to interfere with daily activities and that may prompt medical intervention and/or additional observation.

As used herein, the phrase “severe allergic adverse event” refers to an observed or experienced OIT-treatment-related allergic adverse event that requires hospitalization and/or administration of epinephrine or other life-saving medical intervention.

The term “subject” or “patient” is used synonymously herein to describe a human of any age.

A composition is “substantially free” of a material if the composition contains less than 0.005 wt % of that material or is free of that material.

A patient “tolerates” a dose when the dose is administered to the patient and fully consumed by the patient without any moderate or severe allergic adverse event in response to the dose. A patient is considered to tolerate the dose even if a mild allergic adverse event is observed or experienced. A “highest tolerated dose” is the maximum dose administered to the patient during an oral food challenge that is tolerated by the patient without any moderate or severe allergic adverse event. A “cumulative tolerated dose” is the sum of doses administered to the patient during an oral food challenge up to and including the highest tolerated dose, without any moderate or severe allergic adverse event.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom; delay in progression of the disease; delay in recurrence of the disease; inhibition of the disease; or partially or fully reducing a response or reaction to an allergen.

An “up-dosing phase” refers to a phase of an oral immunotherapy characterized by a series of increasing food allergen doses, beginning with administration of a dose of food allergen lower than the highest dose administered to the patient during the oral immunotherapy, and ending when the highest dose administered to the patient during the oral immunotherapy is achieved.

The terms “weight percentage,” “weight percent,” and “wt %” are used synonymously herein, and refer to the percentage of the listed component of a composition compared to the total weight of the composition.

It is understood that aspects and variations of the invention described herein include “consisting” and/or “consisting essentially of” aspects and variations.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some, or all of the properties of the various embodiments described herein may be combined to form other embodiments of the present invention. The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Egg White Protein Formulations

The egg white protein formulations described herein include a dried egg white (DEW) protein powder, and can be formulated with one or more excipients. Typically, the formulation includes one or more different diluents (preferably two different diluents) and a lubricant. Some egg white protein formulations further include a glidant, although the glidant may also be omitted in certain formulations. For example, the glidant is commonly omitted in lower dose formulations (e.g., formulations with less than about 2.5 wt % of egg white protein). Thus, in some formulations, the egg white protein formulation is substantially free of a glidant or substantially free of colloidal silicon dioxide. The excipients and proportion of excipients may be selected depending on the dose of egg white protein when packaged in a dosage container.

The dried egg white protein powder is typically derived from fowl (preferably from chicken (Gallus domesticus)) egg whites. Dried egg white protein powder is frequently used as a food ingredient, although it can be formulated for use as an oral immunotherapy formulation for treating egg allergies. It is inadvisable to use non-formulated dried egg white protein powder for treating oral immunotherapy because safe treatment requires careful dose administration, particularly at lower doses, that is difficult to consistently obtain without formulation. Dried egg white protein powder generally has a protein content of about 80% to about 85%, although the quantity of dried egg white protein powder added to a formulation lot can be adjusted based on the actual protein content of the dried egg white protein powder, with a concomitant adjustment to the quantity of one or more excipients (such as one of the diluents, such as pregelatinized starch) to obtain the desired dosage levels. For example, the protein content of the dried egg white protein powder can be between about 50 wt % and about 90 wt % (such as about 70 wt % to about 90 wt %, about 75 wt % to about 90 wt %, or about 70 wt % to about 85 wt %). The remaining weight of the dried egg white protein powder may include small amounts of fat, carbohydrates, vitamins, minerals, or other components that are naturally present in the egg white. The dried egg white protein powder may further include residual water.

The dried egg white protein powder is derived from liquid egg whites, generally through spray drying the dried egg whites. Preferably, the dried egg white protein powder is pasteurized, or sterilized (for example, by irradiation), although care should be taken such that the allergenic proteins are not denatured during the manufacturing process. Although spray drying and pasteurization are not expected to denature the proteins, quality control processes (such as determining the potency or relative potency of one or more allergenic proteins such as ovalbumin, ovotransferrin, ovomucoid, and/or lysozyme) can be used to assess the dried egg white protein powder and/or the manufactured egg white protein formulation. Polyacrylamide gel electrophoresis (PAGE), e.g., SDS-PAGE, or an immunoblot can also be used to detect degradation of lysozyme or ovotransferrin, which disappear or become faded from the gel when degraded.

Dried egg white protein powder can be manufactured by separating liquid egg white and egg yolk from broken egg. The pH of the egg white can be adjusted, if necessary, using suitable acids or bases known in the art. Glucose can be removed from the liquid egg white, which can decrease browning of the dried egg white protein powder that can occur through a Maillard reaction. Glucose can be removed, for example, by adding glucose oxidase, catalase, and a hydrogen peroxide solution to the liquid egg white; or through fermentation (for example, by adding yeast to the egg white).

To dry the liquid egg white, the liquid egg white can be passed through a spray dryer. Liquid egg white is atomized through a spray dryer nozzle into a chamber and dried using a heated air stream. The amount of heat and length of exposure are set to dry the liquid egg white into a powder, but not denature the allergenic proteins (e.g., lysozyme, ovalbumin, ovomucoid, and ovotransferrin) within the egg white.

The dried egg white protein powder can be pasteurized after the drying process. Pasteurization kills harmful bacteria, such as Salmonella, but does not denature the allergenic proteins in the dried egg white protein powder. To pasteurize the dried egg white protein powder, the powder can be held at about 50° C. to about 60° C. (and preferably at about 54° C. to about 60° C.) for about 7 days or more. The pasteurization process may last for at least until the dried egg white powder is negative for live Salmonella spp. and/or E. coli (either of which can be assessed, for example, by differential culturing or other suitable means).

The dried egg white protein powder may include materials added during the course of processing the powder, such as glucose oxidase, catalase, hydrogen peroxide, an acid or base used to adjust pH, or a vehicle for any of the components (e.g., salts, buffers, stabilizers, etc.). However, these additives are relatively small, and it is generally preferred that the dried egg white protein powder is substantially free of additives prior to manufacture of the egg white protein formulation, wherein formulation excipients are mixed with the dried egg white protein powder.

As the egg white protein formulations are manufactured by combining and blending powders, the particle size of the dried egg white protein powder was identified as a potential factor that could affect blend uniformity of batch-produced formulations and/or content uniformity of batches of dosage containers, especially at the lowest dosage strengths. Powder particle size can affect the blending process, particle adhesion to carrier excipients or processing equipment surfaces, or segregation following the blending process. The median particle diameter (d₅₀) of particles in the dried egg white protein powder used in the formulations described herein is generally about 30 μm to about 150 μm (such as about 30 μm to about 50 μm, about 50 μm to about 80 μm, about 80 μm to about 120 μm, or about 120 μm to about 150 μm). D₁₀ is typically about 3 μm or higher (and less than the median particle diameter). D₉₀ is typically about 250 μm or lower (and more than the median particle diameter), such as about 200 μm or less, or about 175 μm or less. Particle size distribution metrics (d₅₀, d₉₀, d₁₀) refer to the particle diameter at the referenced percentile (i.e., 50th, 90th, or 10th) by cumulative mass (or cumulative volume, which is equivalent to mass for uniformly dense particles) or by cumulative number. Particle size can be measured using standard laser diffraction techniques (e.g., dynamic laser scattering), wherein the particles are suspended in a solvent that does not dissolve the particles (such as methanol, ethanol, or other suitable solvent), or by sieve analysis.

Excipients included in the egg white protein formulations were selected so that the formulations can be packaged (for example, into capsules, sachets, or other suitable packaging) with an accurate and consistent content of egg white protein. The excipients of the formulations include one or more (and preferably two) different diluents, and a lubricant. In some formulations, a glidant is included. However, in some embodiments, the glidant is omitted from the formulation. For example, in some formulations (such as lower-dose formulations having less than about 5 wt % egg white protein), the egg white protein formulation is free or substantially free of colloidal silicon dioxide. Exemplary diluents included in the formulations include pregelatinized starch and microcrystalline cellulose, or a combination thereof. Magnesium stearate was found to be a suitable lubricant, and is generally included in the formulation. The glidant, if included in the formulation, is preferably colloidal silicon dioxide.

The egg white protein formulations are packaged into dosage containers, and the amount of egg white protein formulation and the concentration of egg white protein in the formulation determines the dose of egg white protein in the dosage containers. The dosage containers are not consumed whole, but instead the contents are removed prior to consumption. The egg white protein formulation, once removed, can be mixed with a food vehicle (such as yogurt, oatmeal, pudding, apple sauce, or other suitable food vehicle) to aid consumption. Because dose accuracy is particularly important when administering an oral immunotherapy, the egg white protein formulations should have good flow properties to ensure an adequate portion of the contents of the container are removed and consumed. The formulations described herein allow the egg white protein formulations in the container to adequately flow out of the container prior to consumption.

Pregelatinized starch and microcrystalline cellulose are particularly useful diluents because they are free-flowing powders that mix with the egg white protein powder. A greater percentage of microcrystalline cellulose is included for lower egg white protein dose formulations (for example, doses of about 10 mg or less) than higher doses because it ensures good flowability and bulk density of the egg white protein formulation for packaging in containers and removal from the containers, such as packaging in and removal from capsules. For example, lower doses (e.g., about 10 mg or less) of the egg white protein formulation may include about 25 wt % to about 60 wt % (such as about 25 wt % to about 30 wt %, about 30 wt % to about 40 wt %, about 40 wt % to about 50 wt %, or about 50 wt % to about 60 wt %) microcrystalline cellulose, whereas higher doses (e.g., more than about 10 mg) of the egg white protein formulation may include about 5 wt % to about 25 wt % (such as about 5 wt % to about 10 wt %, about 10 wt % to about 15 wt %, about 15 wt % to about 20 wt %, or about 20 wt % to about 25 wt %).

Magnesium stearate was found to be a useful lubricant to prevent adhesion of the egg white protein to equipment surfaces during the manufacturing process. Generally, the egg white protein formulation includes about 0.2 wt % to about 2 wt % of magnesium stearate, such as about 0.2 wt % to about 0.4 wt %, about 0.4 wt % to about 0.6 wt %, about 0.6 wt % to about 1 wt %, about 1 wt % to about 1.5 wt %, or about 1.5 wt % to about 2 wt %. In some embodiments, the egg white protein formulation includes about 0.5 wt % magnesium stearate.

A glidant, such as colloidal silicon dioxide, can be included in higher doses of the egg white protein formulation (for example, doses of more than about 6 mg egg white protein or doses of more than about 12 mg egg white protein). Omission of the glidant from larger doses was found to result in a portion of the egg white protein formulation remaining in the container, which would result in under-dosing of the patient. Inclusion of the glidant in the egg white formulation, however, allowed substantially all of the egg white protein formulation to be deliverable from the container when poured out. The addition of colloidal silicon dioxide was found to form soft low-density agglomerates. A high-shear mixing step (such as a conical mill) was one solution discovered to improve the content uniformity of formulations containing colloidal silicon dioxide. Preferably, the high-shear mixing step to disrupt these low-density agglomerates does not change the primary particle size of any blend components. The glidant, such as colloidal silicon dioxide, can therefore be included in the egg white protein formulations, for example in an amount of about 0.2 wt % to about 2 wt % (such as about 0.2 wt % to about 0.4 wt %, about 0.4 wt % to about 0.6 wt %, about 0.6 wt % to about 1 wt %, about 1 wt % to about 1.5 wt %, or about 1.5 wt % to about 2 wt %). Inclusion of the glidant can increase the deliverability of the egg white protein powder from the container in higher doses. In some embodiments, about 95 wt % or more (such as about 96 wt % or more, about 97 wt % or more, about 98 wt % or more, about 99 wt % or more, or about 99.5 wt % or more) of the egg white protein formulation in the container is deliverable from the container. Further, the egg white protein formulation can be reliably delivered from the container. For example, in some embodiments, the dosage container is selected from a batch comprising a plurality of dosage containers, average deliverable egg white protein formulation is about 95 wt % or more (such as about 96 wt % or more, about 97 wt % or more, about 98 wt % or more, about 99 wt % or more, or about 99.5 wt % or more).

For lower doses of the egg white protein formulation (e.g., about 6 mg egg white protein or less), it was found that silicon dioxide interfered with quality control analysis, and in particular quantifying protein concentration. As previously discussed, the amount of egg white protein included in dosage containers should be accurate to minimize risk of accidental overdose, particularly when low doses are administered to a subject. It was further discovered that the glidant that was included in the egg white protein formulations for higher dose amounts to ensure sufficient deliverability from dosage containers could be omitted in the egg white protein formulations used in lower dose amounts while maintaining sufficient deliverability from the container. In some embodiments, about 95 wt % or more (such as about 96 wt % or more, about 97 wt % or more, about 98 wt % or more, about 99 wt % or more, or about 99.5 wt % or more) of the egg white protein formulation in a dosage container comprising 6 mg egg white protein or less (such as between about 0.1 mg and about 6 mg egg white protein) is deliverable from the container even when the egg white protein formulation is free of the colloidal silicon dioxide. Further, the egg white protein formulation can be reliably delivered from the container. For example, in some embodiments, the dosage container is selected from a batch comprising a plurality of dosage containers, average deliverable egg white protein formulation is about 95 wt % or more (such as about 96 wt % or more, about 97 wt % or more, about 98 wt % or more, about 99 wt % or more, or about 99.5 wt % or more).

### Egg White Protein Formulation Doses

The egg white protein formulations described herein are packaged in dosage containers, such as capsules or sachets. During the course of oral immunotherapy, different doses of egg white protein are administered to a patient with an egg allergy, and the dose is selected based on the treatment phase and/or tolerability of the egg white protein. In brief, patients orally ingest increasing amounts of egg white protein during an up-dosing phase (usually through daily administration of the egg white protein formulation, with a periodic dose increase (e.g., a dose increase once every two weeks)), which is followed by a maintenance phase at a higher dose level. Further explanation of an exemplary oral immunotherapy dosing schedule is provided herein. For administration, the egg white protein formulation is removed from the dose containers and orally consumed. For example, a capsule containing an egg white protein formulation is not consumed whole, but instead the egg white protein formulation is removed from the capsule prior to oral administration to the patient. Preferably, the egg white protein formulation is mixed with a food vehicle prior to consumption.

The amount of an egg white protein formulation with a given egg white protein concentration packaged in a dosage container sets the amount of egg white protein (i.e., the dose) in that dose container. Therefore, the dose of a container is a function of the egg white protein concentration in the formulation and the amount of formulation added to the container. The intended doses (i.e., label claim) of egg white protein in a dosage container can rage, for example, from about 0.1 mg to about 600 mg egg white protein, or any amount within this range. By way of example, doses (label claim) for a dosage container can be 0.2 mg, 1 mg, 3 mg, 6, mg, 12 mg, 20 mg, 40 mg, 80 mg, 120 mg, 160 mg, 200 mg, 240 mg, or 300 mg egg white protein.

The amount of egg white protein formulation in the container is limited by the size of the container, but generally ranges from about 100 mg to about 1000 mg, such as about 100 mg to about 250 mg, about 250 mg to about 400 mg, about 400 mg to about 600 mg, or about 600 mg to about 1000 mg. By way of example, in some embodiments about 180 mg of egg white protein formulation is included in a container (such as a capsule). In some embodiments, about 500 mg of egg white protein formulation is included in a container (such as a capsule or sachet). Smaller containers may be more suitable for smaller dose sized. For example, egg white protein formulation to obtain a dose of about 0.1 mg to about 10 mg (or about 0.2 mg to about 6 mg) egg white protein may be included in a container with a capacity of about 180 mg (e.g., a capsule size of 2). Larger doses of egg white protein formulation may be included in larger container sizes, for example a dose of about 12 mg to about 300 mg may be included in a container with a capacity of about 500 mg (e.g., a capsule size of 00). Exemplary capsule sizes can be 000, 00, 0, 1, 2, or 3.

Because the dosage container is not itself ingested (the contents of the container are ingested), the material of the container need not be edible. Nevertheless, it can be useful to have an edible container in the event of inadvertent consumption of the container. The container preferably limits exposure of the contents to moisture or air. Exemplary containers may be a hypromellose-based container (such as a capsule), or a foil lined sachet. The container should be readily openable so that the egg white protein formulation in the container can flow from the container.

In one example, the egg white protein formulation comprises, consist essentially of, or consists of about 0.1 wt % to about 0.3 wt % dried egg white protein powder, about 50 wt % to about 70 wt % of a first diluent (such as pregelatinized starch), about 35 wt % to about 45 wt % of a second diluent (such as microcrystalline cellulose), and about 0.25 wt % to about 0.75 wt % of a lubricant (such as magnesium stearate). In some embodiments, the egg white protein formulation is substantially free of a glidant or substantially free of colloidal silicon dioxide. In some embodiments, the egg white protein formulation comprises about 0.08 wt % to about 0.24 wt % egg white protein. The egg white protein formulation may be packaged in a dosage container (such as a capsule, for example a size 2 capsule) with an intended egg white protein dose (i.e., label claim) of 0.2 mg. The dosage container may contain about 170 mg to about 190 mg of the formulation. Because actual fill amounts of the formulation may vary, the actual dose of egg white protein in the dosage container may be between about 0.15 mg to about 0.25 mg (such as about 0.17 mg to about 0.23 mg, about 0.18 mg to about 0.22 mg, or about 0.19 mg to about 0.21 mg).

In another example, the egg white protein formulation comprises, consist essentially of, or consists of about 0.1 wt % to about 0.3 wt % dried egg white protein powder, about 50 wt % to about 70 wt % of pregelatinized starch, about 35 wt % to about 45 wt % of microcrystalline cellulose, and about 0.25 wt % to about 0.75 wt % of magnesium stearate. In some embodiments, the egg white protein formulation is substantially free of a glidant or substantially free of colloidal silicon dioxide. In some embodiments, the egg white protein formulation comprises about 0.08 wt % to about 0.24 wt % egg white protein. The egg white protein formulation may be packaged in a dosage container (such as a capsule, for example a size 2 capsule) with an intended egg white protein dose (i.e., label claim) of 0.2 mg. The dosage container may contain about 170 mg to about 190 mg of the formulation. Because actual fill amounts of the formulation may vary, the actual dose of egg white protein in the dosage container may be between about 0.15 mg to about 0.25 mg (such as about 0.17 mg to about 0.23 mg, about 0.18 mg to about 0.22 mg, or about 0.19 mg to about 0.21 mg).

In another example, the egg white protein formulation comprises, consist essentially of, or consists of about 0.5 wt % to about 0.8 wt % dried egg white protein powder, about 50 wt % to about 70 wt % of a first diluent (such as pregelatinized starch), about 35 wt % to about 45 wt % of a second diluent (such as microcrystalline cellulose), and about 0.25 wt % to about 0.75 wt % of a lubricant (such as magnesium stearate). In some embodiments, the egg white protein formulation is substantially free of a glidant or substantially free of colloidal silicon dioxide. In some embodiments, the egg white protein formulation comprises about 0.4 wt % to about 0.64 wt % egg white protein. The egg white protein formulation may be packaged in a dosage container (such as a capsule, for example a size 2 capsule) with an intended egg white protein dose (i.e., label claim) of 1 mg. The dosage container may contain about 170 mg to about 190 mg of the formulation. Because actual fill amounts of the formulation may vary, the actual dose of egg white protein in the dosage container may be between about 0.75 mg to about 1.25 mg (such as about 0.85 mg to about 1.15 mg, about 0.9 mg to about 1.1 mg, or about 0.95 mg to about 1.05 mg).

In another example, the egg white protein formulation comprises, consist essentially of, or consists of about 0.5 wt % to about 0.8 wt % dried egg white protein powder, about 50 wt % to about 70 wt % of pregelatinized starch, about 35 wt % to about 45 wt % of microcrystalline cellulose, and about 0.25 wt % to about 0.75 wt % of magnesium stearate. In some embodiments, the egg white protein formulation is substantially free of a glidant or substantially free of colloidal silicon dioxide. In some embodiments, the egg white protein formulation comprises about 0.4 wt % to about 0.64 wt % egg white protein. The egg white protein formulation may be packaged in a dosage container (such as a capsule, for example a size 2 capsule) with an intended egg white protein dose (i.e., label claim) of 1 mg. The dosage container may contain about 170 mg to about 190 mg of the formulation. Because actual fill amounts of the formulation may vary, the actual dose of egg white protein in the dosage container may be between about 0.75 mg to about 1.25 mg (such as about 0.85 mg to about 1.15 mg, about 0.9 mg to about 1.1 mg, or about 0.95 mg to about 1.05 mg).

In another example, the egg white protein formulation comprises, consist essentially of, or consists of about 1.8 wt % to about 2.4 wt % dried egg white protein powder, about 50 wt % to about 70 wt % of a first diluent (such as pregelatinized starch), about 35 wt % to about 45 wt % of a second diluent (such as microcrystalline cellulose), and about 0.25 wt % to about 0.75 wt % of a lubricant (such as magnesium stearate). In some embodiments, the egg white protein formulation is substantially free of a glidant or substantially free of colloidal silicon dioxide. In some embodiments, the egg white protein formulation comprises about 1.44 wt % to about 1.92 wt % egg white protein. The egg white protein formulation may be packaged in a dosage container (such as a capsule, for example a size 2 capsule) with an intended egg white protein dose (i.e., label claim) of 3 mg. The dosage container may contain about 170 mg to about 190 mg of the formulation. Because actual fill amounts of the formulation may vary, the actual dose of egg white protein in the dosage container may be between about 2.25 mg to about 3.75 mg (such as about 2.55 mg to about 3.45 mg, about 2.7 mg to about 3.3 mg, or about 2.85 mg to about 3.15 mg).

In another example, the egg white protein formulation comprises, consist essentially of, or consists of about 1.8 wt % to about 2.4 wt % dried egg white protein powder, about 50 wt % to about 70 wt % of pregelatinized starch, about 35 wt % to about 45 wt % of microcrystalline cellulose, and about 0.25 wt % to about 0.75 wt % of magnesium stearate. In some embodiments, the egg white protein formulation is substantially free of a glidant or substantially free of colloidal silicon dioxide. In some embodiments, the egg white protein formulation comprises about 1.44 wt % to about 1.92 wt % egg white protein. The egg white protein formulation may be packaged in a dosage container (such as a capsule, for example a size 2 capsule) with an intended egg white protein dose (i.e., label claim) of 3 mg. The dosage container may contain about 170 mg to about 190 mg of the formulation. Because actual fill amounts of the formulation may vary, the actual dose of egg white protein in the dosage container may be between about 2.25 mg to about 3.75 mg (such as about 2.55 mg to about 3.45 mg, about 2.7 mg to about 3.3 mg, or about 2.85 mg to about 3.15 mg).

In another example, the egg white protein formulation comprises, consist essentially of, or consists of about 4 wt % to about 4.5 wt % dried egg white protein powder, about 45 wt % to about 65 wt % of a first diluent (such as pregelatinized starch), about 35 wt % to about 45 wt % of a second diluent (such as microcrystalline cellulose), and about 0.25 wt % to about 0.75 wt % of a lubricant (such as magnesium stearate). In some embodiments, the egg white protein formulation is substantially free of a glidant or substantially free of colloidal silicon dioxide. In some embodiments, the egg white protein formulation comprises about 1.44 wt % to about 1.92 wt % egg white protein. The egg white protein formulation may be packaged in a dosage container (such as a capsule, for example a size 2 capsule) with an intended egg white protein dose (i.e., label claim) of 6 mg. The dosage container may contain about 170 mg to about 190 mg of the formulation. Because actual fill amounts of the formulation may vary, the actual dose of egg white protein in the dosage container may be between about 4.5 mg to about 7.5 mg (such as about 5.1 mg to about 6.9 mg, about 5.4 mg to about 6.6 mg, about 5.7 mg to about 6.3 mg, or about 5.8 mg to about 6.2 mg).

In another example, the egg white protein formulation comprises, consist essentially of, or consists of about 4 wt % to about 4.5 wt % dried egg white protein powder, about 45 wt % to about 65 wt % of pregelatinized starch, about 35 wt % to about 45 wt % of microcrystalline cellulose, and about 0.25 wt % to about 0.75 wt % of magnesium stearate. In some embodiments, the egg white protein formulation is substantially free of a glidant or substantially free of colloidal silicon dioxide. In some embodiments, the egg white protein formulation comprises about 1.44 wt % to about 1.92 wt % egg white protein. The egg white protein formulation may be packaged in a dosage container (such as a capsule, for example a size 2 capsule) with an intended egg white protein dose (i.e., label claim) of 6 mg. The dosage container may contain about 170 mg to about 190 mg of the formulation. Because actual fill amounts of the formulation may vary, the actual dose of egg white protein in the dosage container may be between about 4.5 mg to about 7.5 mg (such as about 5.1 mg to about 6.9 mg, about 5.4 mg to about 6.6 mg, about 5.7 mg to about 6.3 mg, or about 5.8 mg to about 6.2 mg).

In another example, the egg white protein formulation comprises, consist essentially of, or consists of about 2.5 wt % to about 3.5 wt % dried egg white protein powder, about 70 wt % to about 85 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 2 wt % to about 2.8 wt % egg white protein. The egg white protein formulation may be packaged in a dosage container (such as a capsule, for example a size 00 capsule) with an intended egg white protein dose (i.e., label claim) of 12 mg. The dosage container may contain about 480 mg to about 520 mg of the formulation. Because actual fill amounts of the formulation may vary, the actual dose of egg white protein in the dosage container may be between about 9 mg to about 15 mg (such as about 10.2 mg to about 13.8 mg, about 10.8 mg to about 13.2 mg, about 11.4 mg to about 12.6 mg, or about 11.7 mg to about 12.3 mg).

In another example, the egg white protein formulation comprises, consist essentially of, or consists of about 2.5 wt % to about 3.5 wt % dried egg white protein powder, about 70 wt % to about 85 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 2 wt % to about 2.8 wt % egg white protein. The egg white protein formulation may be packaged in a dosage container (such as a capsule, for example a size 00 capsule) with an intended egg white protein dose (i.e., label claim) of 12 mg. The dosage container may contain about 480 mg to about 520 mg of the formulation. Because actual fill amounts of the formulation may vary, the actual dose of egg white protein in the dosage container may be between about 9 mg to about 15 mg (such as about 10.2 mg to about 13.8 mg, about 10.8 mg to about 13.2 mg, about 11.4 mg to about 12.6 mg, or about 11.7 mg to about 12.3 mg).

In another example, the egg white protein formulation comprises, consist essentially of, or consists of about 4.5 wt % to about 5.5 wt % dried egg white protein powder, about 70 wt % to about 85 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 3.6 wt % to about 4.4 wt % egg white protein. The egg white protein formulation may be packaged in a dosage container (such as a capsule, for example a size 00 capsule) with an intended egg white protein dose (i.e., label claim) of 20 mg. The dosage container may contain about 480 mg to about 520 mg of the formulation. Because actual fill amounts of the formulation may vary, the actual dose of egg white protein in the dosage container may be between about 15 mg to about 25 mg (such as about 17 mg to about 23 mg, about 18 mg to about 22 mg, about 19 mg to about 21 mg, or about 19.5 mg to about 20.5 mg).

In another example, the egg white protein formulation comprises, consist essentially of, or consists of about 4.5 wt % to about 5.5 wt % dried egg white protein powder, about 70 wt % to about 85 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 3.6 wt % to about 4.4 wt % egg white protein. The egg white protein formulation may be packaged in a dosage container (such as a capsule, for example a size 00 capsule) with an intended egg white protein dose (i.e., label claim) of 20 mg. The dosage container may contain about 480 mg to about 520 mg of the formulation. Because actual fill amounts of the formulation may vary, the actual dose of egg white protein in the dosage container may be between about 15 mg to about 25 mg (such as about 17 mg to about 23 mg, about 18 mg to about 22 mg, about 19 mg to about 21 mg, or about 19.5 mg to about 20.5 mg).

In another example, the egg white protein formulation comprises, consist essentially of, or consists of about 8 wt % to about 12 wt % dried egg white protein powder, about 65 wt % to about 85 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 6.4 wt % to about 9.6 wt % egg white protein. The egg white protein formulation may be packaged in a dosage container (such as a capsule, for example a size 00 capsule) with an intended egg white protein dose (i.e., label claim) of 40 mg. The dosage container may contain about 480 mg to about 520 mg of the formulation. Because actual fill amounts of the formulation may vary, the actual dose of egg white protein in the dosage container may be between about 30 mg to about 50 mg (such as about 34 mg to about 46 mg, about 36 mg to about 44 mg, about 38 mg to about 42 mg, or about 39 mg to about 41 mg).

In another example, the egg white protein formulation comprises, consist essentially of, or consists of about 8 wt % to about 12 wt % dried egg white protein powder, about 65 wt % to about 85 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 6.4 wt % to about 9.6 wt % egg white protein. The egg white protein formulation may be packaged in a dosage container (such as a capsule, for example a size 00 capsule) with an intended egg white protein dose (i.e., label claim) of 40 mg. The dosage container may contain about 480 mg to about 520 mg of the formulation. Because actual fill amounts of the formulation may vary, the actual dose of egg white protein in the dosage container may be between about 30 mg to about 50 mg (such as about 34 mg to about 46 mg, about 36 mg to about 44 mg, about 38 mg to about 42 mg, or about 39 mg to about 41 mg).

In another example, the egg white protein formulation comprises, consist essentially of, or consists of about 16 wt % to about 24 wt % dried egg white protein powder, about 55 wt % to about 75 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 12.8 wt % to about 19.2 wt % egg white protein. The egg white protein formulation may be packaged in a dosage container (such as a capsule, for example a size 00 capsule) with an intended egg white protein dose (i.e., label claim) of 80 mg. The dosage container may contain about 480 mg to about 520 mg of the formulation. Because actual fill amounts of the formulation may vary, the actual dose of egg white protein in the dosage container may be between about 60 mg to about 100 mg (such as about 68 mg to about 92 mg, about 72 mg to about 88 mg, about 76 mg to about 84 mg, or about 78 mg to about 82 mg).

In another example, the egg white protein formulation comprises, consist essentially of, or consists of about 16 wt % to about 24 wt % dried egg white protein powder, about 55 wt % to about 75 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 12.8 wt % to about 19.2 wt % egg white protein. The egg white protein formulation may be packaged in a dosage container (such as a capsule, for example a size 00 capsule) with an intended egg white protein dose (i.e., label claim) of 80 mg. The dosage container may contain about 480 mg to about 520 mg of the formulation. Because actual fill amounts of the formulation may vary, the actual dose of egg white protein in the dosage container may be between about 60 mg to about 100 mg (such as about 68 mg to about 92 mg, about 72 mg to about 88 mg, about 76 mg to about 84 mg, or about 78 mg to about 82 mg).

In another example, the egg white protein formulation comprises, consist essentially of, or consists of about 24 wt % to about 36 wt % dried egg white protein powder, about 45 wt % to about 65 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 19.2 wt % to about 28.8 wt % egg white protein. The egg white protein formulation may be packaged in a dosage container (such as a capsule, for example a size 00 capsule) with an intended egg white protein dose (i.e., label claim) of 120 mg. The dosage container may contain about 480 mg to about 520 mg of the formulation. Because actual fill amounts of the formulation may vary, the actual dose of egg white protein in the dosage container may be between about 90 mg to about 150 mg (such as about 102 mg to about 138 mg, about 108 mg to about 132 mg, about 114 mg to about 126 mg, or about 117 mg to about 123 mg).

In another example, the egg white protein formulation comprises, consist essentially of, or consists of about 24 wt % to about 36 wt % dried egg white protein powder, about 45 wt % to about 65 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 19.2 wt % to about 28.8 wt % egg white protein. The egg white protein formulation may be packaged in a dosage container (such as a capsule, for example a size 00 capsule) with an intended egg white protein dose (i.e., label claim) of 120 mg. The dosage container may contain about 480 mg to about 520 mg of the formulation. Because actual fill amounts of the formulation may vary, the actual dose of egg white protein in the dosage container may be between about 90 mg to about 150 mg (such as about 102 mg to about 138 mg, about 108 mg to about 132 mg, about 114 mg to about 126 mg, or about 117 mg to about 123 mg).

In another example, the egg white protein formulation comprises, consist essentially of, or consists of about 32 wt % to about 38 wt % dried egg white protein powder, about 35 wt % to about 55 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 25.6 wt % to about 30.4 wt % egg white protein. The egg white protein formulation may be packaged in a dosage container (such as a capsule, for example a size 00 capsule) with an intended egg white protein dose (i.e., label claim) of 160 mg. The dosage container may contain about 480 mg to about 520 mg of the formulation. Because actual fill amounts of the formulation may vary, the actual dose of egg white protein in the dosage container may be between about 120 mg to about 200 mg (such as about 136 mg to about 184 mg, about 144 mg to about 176 mg, about 152 mg to about 168 mg, or about 156 mg to about 154 mg).

In another example, the egg white protein formulation comprises, consist essentially of, or consists of about 32 wt % to about 38 wt % dried egg white protein powder, about 35 wt % to about 55 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 25.6 wt % to about 30.4 wt % egg white protein. The egg white protein formulation may be packaged in a dosage container (such as a capsule, for example a size 00 capsule) with an intended egg white protein dose (i.e., label claim) of 160 mg. The dosage container may contain about 480 mg to about 520 mg of the formulation. Because actual fill amounts of the formulation may vary, the actual dose of egg white protein in the dosage container may be between about 120 mg to about 200 mg (such as about 136 mg to about 184 mg, about 144 mg to about 176 mg, about 152 mg to about 168 mg, or about 156 mg to about 154 mg).

In another example, the egg white protein formulation comprises, consist essentially of, or consists of about 40 wt % to about 60 wt % dried egg white protein powder, about 25 wt % to about 45 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 32 wt % to about 48 wt % egg white protein. The egg white protein formulation may be packaged in a dosage container (such as a capsule, for example a size 00 capsule) with an intended egg white protein dose (i.e., label claim) of 200 mg. The dosage container may contain about 480 mg to about 520 mg of the formulation. Because actual fill amounts of the formulation may vary, the actual dose of egg white protein in the dosage container may be between about 150 mg to about 250 mg (such as about 170 mg to about 230 mg, about 180 mg to about 220 mg, about 190 mg to about 210 mg, or about 195 mg to about 205 mg).

In another example, the egg white protein formulation comprises, consist essentially of, or consists of about 40 wt % to about 60 wt % dried egg white protein powder, about 25 wt % to about 45 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 32 wt % to about 48 wt % egg white protein. The egg white protein formulation may be packaged in a dosage container (such as a capsule, for example a size 00 capsule) with an intended egg white protein dose (i.e., label claim) of 200 mg. The dosage container may contain about 480 mg to about 520 mg of the formulation. Because actual fill amounts of the formulation may vary, the actual dose of egg white protein in the dosage container may be between about 150 mg to about 250 mg (such as about 170 mg to about 230 mg, about 180 mg to about 220 mg, about 190 mg to about 210 mg, or about 195 mg to about 205 mg).

In another example, the egg white protein formulation comprises, consist essentially of, or consists of about 50 wt % to about 70 wt % dried egg white protein powder, about 15 wt % to about 35 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 40 wt % to about 56 wt % egg white protein. The egg white protein formulation may be packaged in a dosage container (such as a capsule, for example a size 00 capsule) with an intended egg white protein dose (i.e., label claim) of 240 mg. The dosage container may contain about 480 mg to about 520 mg of the formulation. Because actual fill amounts of the formulation may vary, the actual dose of egg white protein in the dosage container may be between about 180 mg to about 300 mg (such as about 204 mg to about 276 mg, about 216 mg to about 264 mg, about 228 mg to about 252 mg, or about 234 mg to about 246 mg).

In another example, the egg white protein formulation comprises, consist essentially of, or consists of about 50 wt % to about 70 wt % dried egg white protein powder, about 15 wt % to about 35 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 40 wt % to about 56 wt % egg white protein. The egg white protein formulation may be packaged in a dosage container (such as a capsule, for example a size 00 capsule) with an intended egg white protein dose (i.e., label claim) of 240 mg. The dosage container may contain about 480 mg to about 520 mg of the formulation. Because actual fill amounts of the formulation may vary, the actual dose of egg white protein in the dosage container may be between about 180 mg to about 300 mg (such as about 204 mg to about 276 mg, about 216 mg to about 264 mg, about 228 mg to about 252 mg, or about 234 mg to about 246 mg).

In another example, the egg white protein formulation comprises, consist essentially of, or consists of about 60 wt % to about 85 wt % dried egg white protein powder, about 5 wt % to about 25 wt % of a first diluent (such as pregelatinized starch), about 10 wt % to about 20 wt % of a second diluent (such as microcrystalline cellulose), about 0.25 wt % to about 0.75 wt % of a lubricant (such as magnesium stearate), and about 0.25 wt % to about 0.75 wt % of a glidant (such as colloidal silicon dioxide). In some embodiments, the egg white protein formulation comprises about 48 wt % to about 68 wt % egg white protein. The egg white protein formulation may be packaged in a dosage container (such as a capsule, for example a size 00 capsule, or a sachet) with an intended egg white protein dose (i.e., label claim) of 300 mg. The dosage container may contain about 480 mg to about 520 mg of the formulation. Because actual fill amounts of the formulation may vary, the actual dose of egg white protein in the dosage container may be between about 225 mg to about 375 mg (such as about 255 mg to about 345 mg, about 270 mg to about 330 mg, about 285 mg to about 315 mg, or about 292 mg to about 308 mg).

In another example, the egg white protein formulation comprises, consist essentially of, or consists of about 60 wt % to about 85 wt % dried egg white protein powder, about 5 wt % to about 25 wt % of pregelatinized starch, about 10 wt % to about 20 wt % of microcrystalline cellulose, about 0.25 wt % to about 0.75 wt % of magnesium stearate, and about 0.25 wt % to about 0.75 wt % of colloidal silicon dioxide. In some embodiments, the egg white protein formulation comprises about 48 wt % to about 68 wt % egg white protein. The egg white protein formulation may be packaged in a dosage container (such as a capsule, for example a size 00 capsule, or a sachet) with an intended egg white protein dose (i.e., label claim) of 300 mg. The dosage container may contain about 480 mg to about 520 mg of the formulation. Because actual fill amounts of the formulation may vary, the actual dose of egg white protein in the dosage container may be between about 225 mg to about 375 mg (such as about 255 mg to about 345 mg, about 270 mg to about 330 mg, about 285 mg to about 315 mg, or about 292 mg to about 308 mg).

The egg white protein formulation, in one aspect, comprises the major egg white allergens Gal d 1, Gal d 2, Gal d 3, and Gal d 4. The amount of each individual allergen may be expressed as a percentage of the total protein in the egg white protein formulation. In one example, a dose of the egg white protein formulation with a label claim of 300 mg comprises approximately 300 mg egg white protein and 78% Gal d 2, which means the dose comprises about 234 mg of Gal d 2. It is understood in the art that egg white proteins may be heavily glycosylated. Therefore, the mass of egg white allergens means the mass of the isolated proteins, which may include a substantial portion of carbohydrates. Ensuring the egg white protein formulation has a consistent and known quantity of the major egg white allergens is important to ensure the suitability of the composition for use in oral immunotherapy of egg allergy.

In some embodiments, the egg white protein formulation comprises between about 5% and about 20% Gal d 1, such as any of about 5% to about 10%, about 10% to about 15%, about 15% to about 20%, about 5% to about 15%, or about 10% to about 20% Gal d 1 as compared to the total egg white protein mass.

In some embodiments, the egg white protein formulation comprises between about 45% to about 90% Gal d 2, such as any of about 45% to about 50%, about 50% to about 60%, about 60% to about 70%, about 70% to about 80%, about 80% to about 90%, about 45% to about 60%, about 60% to about 80%, or about 70% to about 90% Gal d 2 as compared to the total egg white protein mass.

In some embodiments, the egg white protein formulation comprises between about 1% and about 20% Gal d 3, such as any of about 1% to about 5%, about 5% to about 10%, about 10% to about 15%, about 15% to about 20%, about 5% to about 15% Gal d 3 as compared to the total egg white protein mass.

In some embodiments, the egg white protein formulation comprises between about 0.1% and about 10% Gal d 4, such as any of about 0.1% to about 3%, about 3% to about 6%, about 6% to about 9%, about 7% to about 10% Gal d 4 as compared to the total egg white protein mass.

### Methods of Making Egg White Protein Formulations

The method of manufacturing the egg white protein formulations can impact the batch uniformity of a manufactured lot of formulation, the content uniformity of a batch of dosage containers, or the deliverability of the egg white protein formulation from the containers. Because the doses of egg white protein administered during the course of oral immunotherapy can have a wide range (e.g., from about 0.2 mg to about 300 mg), and dosing consistency is important to reduce adverse reactions related to treatment, the formulation manufacturing methods for the different doses was designed to produce dosage containers with consistent doses.

To produce consistent doses, the specific methods of making the formulation can differ between doses. Process development efforts to optimize blend uniformity and content uniformity were focused on the lower doses (e.g., doses less about 3 mg), and process development efforts to ensure adequate formulation flowability were focused on higher doses (e.g., about 300 mg). Processes were also developed for medium-dose strengths to adequately balance the desirable characteristics of the final product.

The methods described herein also overcome challenges that are presented with scaling up manufacturing processes. Ensuring batch uniformity with larger formulation amounts can be challenging, and the methods described herein overcome these challenges. The manufacturing methods can be used even when manufacturing egg white protein formulation lots of about 5 kg or more (such as about 5 kg to about 100 km, for example about 5 kg to about 7 kg, about 7 kg to about 10 kg, about 10 kg to about 15 kg, about 15 kg to about 25 kg, about 25 kg to about 50 kg, or about 50 kg to about 100 kg).

Manufacturing steps such as mixing (for example using a tumble blender or a conical mill), passing one or more powders or mixtures through a mesh screen, and/or serial dilution when adding a diluent to a mixture are used during the manufacturing processes. The specific steps used can depend on the egg white protein concentration in the formulation or the label claim of a dosage form (i.e., the intended amount of egg white protein formulation for a dosage form). Generally, one or more higher-shear force mixing steps (higher shear force relative to other steps in the manufacturing process) will be incorporated into the manufacturing process sequence to uniformly disperse the dried egg white protein powder into the mixture. The higher shear force mixing also disperses the soft low-density agglomerates of colloidal silicon dioxide, if present in the formulation, which are typically observed in colloidal silicon dioxide raw materials. The use of the higher force mixing is not intended to reduce the primary particle size of any mixture components.

After the formulation has been manufactured, the quality of one or more of the allergenic egg white proteins (e.g., ovalbumin, ovomucoid, ovotransferrin, and/or lysozyme) in the formulation and/or the blend uniformity of the formulation can be assessed. Quality of the one or more allergenic proteins can be assessed, for example, by characterizing one or more of ovalbumin, ovomucoid, ovotransferrin, and/or lysozyme (for example, by determining an amount or relative amount of one or more of the proteins, or a potency or a relative potency of one or more of the proteins). Blend uniformity of the formulation can be assessed by determining a protein content of the formulation, which can be compared to an intended protein content (e.g., a label claim or intended concentration) of the formulation. For example, determining blend uniformity of a formulation can include identifying an intended protein content associated with a formulation, measuring the protein content in a sample from the formulation, and comparing the measured protein content with the intended protein content. The blend uniformity can be measured using a plurality of samples from the formulation (e.g., 2, 3, 4, 5, 6, 7, 8, 9, 10 or more samples), and an average blend uniformity and/or relative standard deviation (RSD) can be measured. By using the methods of manufacturing described herein, the blend uniformity RSD of a plurality of samples from the egg white protein formulation is about 15% or less (e.g., about 0.5% to about 15%, or about 1% to about 10%, or about 5% or less (e.g., about 1% to about 5%, or about 2% to about 5%).

The manufactured formulation can be packaged in dosage containers, such as capsules or sachets, in a predetermined amount. The amount of formulation added to each dosage container is intended to provide the amount of egg white protein equal to the label claim for the dosage container. Due to real-world variations that occur during manufacturing and packaging, it is possible there may be some deviation from the label claim and the actual amount of egg white protein in the individual dosage container. Therefore, the content uniformity of a manufactured lot of dosage containers can be measured. Content uniformity is based on the deliverable protein content of the formulation from the dosage container. The dosage containers are associated with an intended amount of egg white protein in the dosage container. Thus, determining content uniformity for a plurality of dosage containers can include identifying an intended protein content associated with the plurality of dosage containers, measuring the deliverable protein content from a sample (e.g., 1, 2, 3, 4, 5, 6, 7, 8, 9, 10, or more dosage containers) taken from the plurality of dosage containers, and comparing the measured protein content to the intended protein content. When a plurality of dosage containers samples are taken from the plurality an average content uniformity and/or content uniformity RSD can be determined. By using the methods of manufacturing described herein, the content uniformity RSD of a plurality of dosage containers is about 12% or less (e.g., about 4% to about 12%, or about 5% to about 12%).

### Low-Dose Manufacturing Processes

Blend uniformity and content uniformity are generally considered to be at greater risks for low dosage strengths corresponding to a low percentage of drug substance in the blend. These lower doses are generally about 12 mg egg white protein or less in a dosage container, although larger doses can be produced using a similar methodology. The formulation manufacturing process generally produces a formulation having about 0.05 wt % to about 4 wt % of egg white protein (such as between about 0.1 wt % to about 0.7 wt %, about 0.7 wt % to about 1.5 wt %, about 1.5 wt % to about 2.5 wt %, or about 2.5 wt % to about 4 wt % egg white protein).

The manufactured egg white protein formulation generally includes two diluents (for example pregelatinized starch and microcrystalline cellulose). One or more of the diluents is mixed with the egg white protein powder in a step-wise manner (i.e., a serial dilution of the egg white protein powder), with the egg white protein powder and a portion of (but not all) the diluent being mixed before an additional amount of the diluent is added to the mixture. Passing the first mixture containing the egg white protein power and the first portion of the diluent through a mesh screen prior to further serial dilution enhances blend uniformity of the fully manufactured egg white protein formulation.

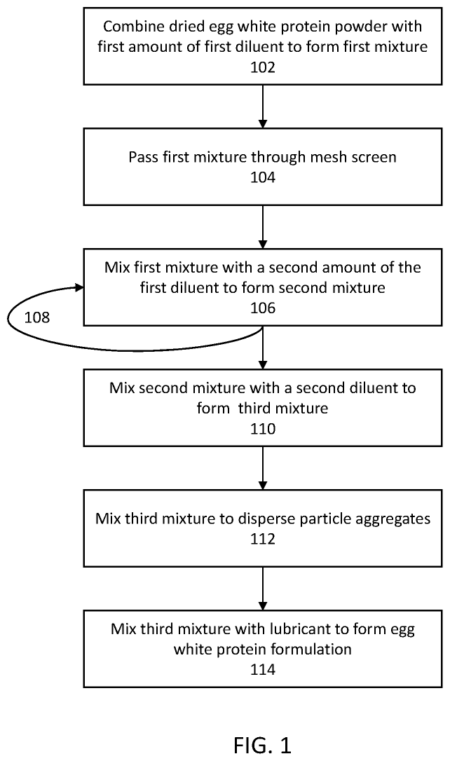

As shown in FIG. 1, dried egg white protein powder is combined with a first amount of a first diluent (e.g., pregelatinized starch) at step 102. The dried egg white protein powder and the first amount of the first diluent can be added to any suitable container, such as a bin or a bag, and can be mixed together before proceeding to the next step. Mixing can be performed, for example, by shaking the container, or by using an impeller, a blender (such as a tumble blender), or any other suitable device.

Once the dried egg white protein powder is combined with the first amount of the first diluent to form a first mixture, the first mixture is passed through a mesh screen (see step 104 of FIG. 1). The size of the mesh screen is generally about 250 μm to about 850 μm (such as about 300 μm to about 710 μm, or about 425 μm to about 600 μm). The size of the mesh screen refers to the average size of the openings in the mesh screen.

Once the first mixture has passed through the mesh screen, the mixture can be further diluted by mixing the first mixture with an additional amount of the first diluent to form a second mixture, as shown at step 106 of FIG. 1. Alternatively, the first mixture and a portion of the additional amount of the first diluent can be co-sieved through the mesh screen corresponding to step 104 of FIG. 1. The first mixture and the additional amount of the first diluent can be mixed by shaking the combined components, or by using an impeller, a blender (such as a tumble blender), or any other suitable device. The additional amount of the first diluent can be added in a step-wise manner to serially dilute the mixture, as indicated by arrow 108 in FIG. 1. For example, first mixture can be mixed with a second amount of the first diluent in a plurality of iterative sub-steps. The sub-steps can include adding a portion of the second amount of the first diluent to the first mixture, and mixing the portion of the second amount of the first diluent and the first mixture. Another portion of the second amount of the first diluent can then be added, which is further mixed. These dilution sub-steps can be performed 1, 2, 3, 4, 5 or more times until the desired amount of the first diluent has been mixed with the dried egg white protein powder. Optionally, the mixture can be passed through a mesh screen before adding an additional portion of the second amount of the first diluent.

Alternatively, the first mixture and the second amount of the first diluent can be mixed together by continuously mixing the first mixture while the second amount of the first diluent is added to the mixture. That is, instead of step-wise adding of the second amount of the first diluent to the first mixture to form the second mixture, the mixture is simultaneously mixed as the second amount of the first diluent is added.

Once the second mixture is formed, the second mixture can be mixed with a second diluent (for example, microcrystalline cellulose) to form a third mixture, as shown in step 110 of FIG. 1. The second mixture and the second diluent can be mixed by shaking the combined components, or by using an impeller, a blender (such as a tumble blender), or any other suitable device. The mixture is also subjected to mixing at a higher shear force than used to form the previous mixtures, as shown in step 112. The higher-shear force mixing may occur after an initial mixing of the second mixture with the second diluent, or can be the mixing force for the mixing of the second mixture with the second diluent. This can be performed, for example, using a conical mill (which may be equipped, for example, using a round mill impeller or a square mill impeller) or other suitable device. In some embodiments, the second mixture and the second diluent are mixed using the higher shear force mixing to initially form the third mixture, and in some embodiments the second mixture and the second diluent are pre-mixed to form the third mixture before the third mixture is subjected to higher-shear force mixing. The higher-shear force mixing increases uniform dispersion of the dried egg white protein powder in the formulation and disperses soft, low-density agglomerates that might form. The use of the higher shear force mixing is not intended to reduce the primary particle size of any mixture components, but only to dissociate agglomerates of particles.

A lubricant (for example, magnesium stearate) is added to the mixture to form the egg white protein formulation, as shown in step 114. The lubricant can be added to any of the mixture during the manufacturing process, but is at some point mixed with the mixture containing the dried egg white protein powder, the first diluent and the second diluent. For example, the lubricant can be mixed with the third mixture before, after, or during the higher-shear force mixing step. A further amount of one or more of the diluents (e.g., the first diluent, such as pregelatinized starch) may be co-added to the mixture with the lubricant. Additionally, the third mixture may be mixed with the lubricant (and optionally an additional amount of the first diluent) and then mixed with an additional amount of the third mixture. In an exemplary embodiment, the third mixture, an additional amount of the first diluent, and the lubricant are mixed, passed through a mesh screen, and then mixed with an additional amount of the third mixture to form the egg white protein formulation. In some embodiments, the lubricant and the additional amount of the first diluent are mixed (and, optionally, the mixture passed through a mesh screen) before being mixed with the mixture containing the egg white protein.

Optionally, the egg white protein formulation can be passed through a mesh screen. The size of the mesh screen is generally about 250 μm to about 850 μm (such as about 300 μm to about 710 μm, or about 425 μm to about 600 μm).

A batch of egg white protein formulation produced using these methods can be assayed for blend uniformity or quality characteristics as further described herein. In some embodiments, the methods further comprise assaying the blend uniformity and/or quality characteristics of the egg white protein formulation.

In an exemplary method of making an egg white protein formulation, the method can include (a) mixing dried egg white protein powder with a first amount of a first diluent (such as pregelatinized starch) to form a first mixture; (b) co-sieving the first mixture with a second portion of the first diluent through a mesh screen; (c) mixing the first mixture with the second portion of the first diluent to form a second mixture after steps (a) and (b); (d) mixing the second mixture with a third portion of the first diluent to form a third mixture; (e) mixing the third mixture with a fourth portion of the first diluent to form a fourth mixture; (f) mixing the fourth mixture with a fifth portion of the first diluent to form a fifth mixture; (g) mixing the fifth mixture with a second diluent (such as microcrystalline cellulose) to form a sixth mixture; (h) mixing the a first portion of sixth mixture with a lubricant (such as magnesium stearate), and optionally a sixth portion of the first diluent, to form a seventh mixture; (j) passing the seventh mixture through a mesh screen; and (k) mixing the screened mixture of (j) with a second portion of the sixth mixture to form the egg white protein formulation. Step (g) may comprise two mixing sub-steps, wherein one mixing sub-step is at a higher shear force than the other mixing sub-step. In an exemplary embodiment, step (g) optionally comprises two sub-steps, wherein the first sub-step is at a lower shear force than the second sub-step (such as with a tumble blender), and the second sub-step is at a higher shear force than the first sub-step (such as with a conical mill). This method of manufacturing the formulation is particularly useful for formulations used to manufacture lower-dose dosage containers containing the formulation, such as doses of about 0.1 mg to about 12 mg (such as about 0.2 mg, about 1 mg, about 3 mg, about 6 mg, or about 12 mg dosage containers, or any dosage therebetween). The formulation may have, for example, about 0.05 wt % to about 2.5% of egg white protein. In some embodiments, the formulation is free or substantially free of a glidant. In some embodiments, the formulation is free or substantially free of colloidal silicon dioxide). A batch of egg white protein formulation produced using these methods can be assayed for blend uniformity or quality characteristics as further described herein. In some embodiments, the methods further comprise assaying the blend uniformity and/or quality characteristics of the egg white protein formulation.

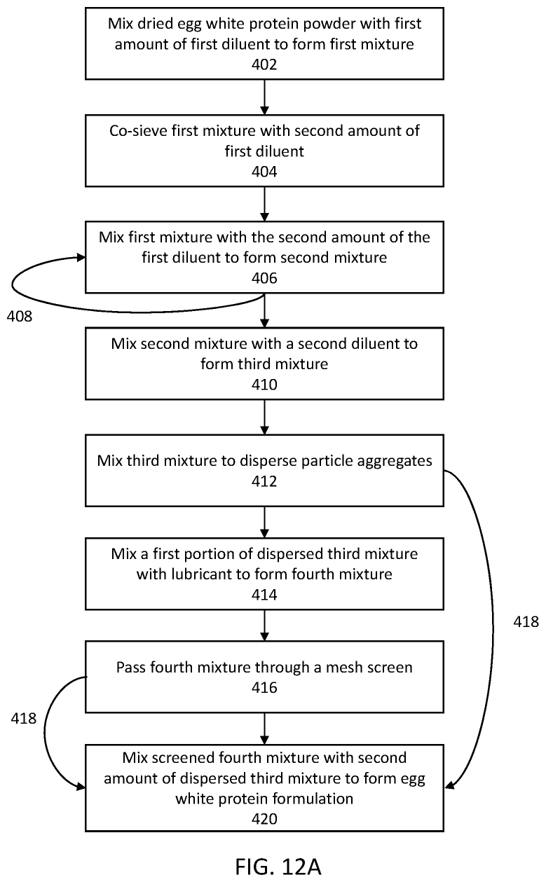

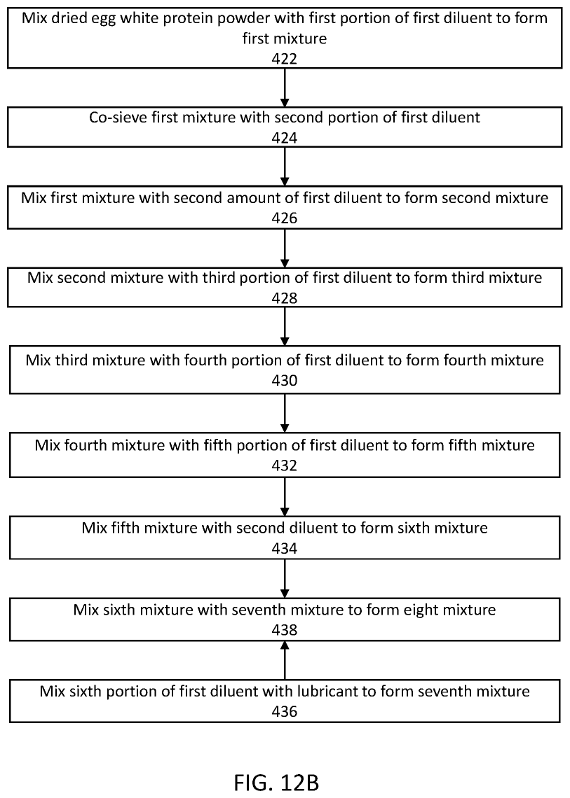

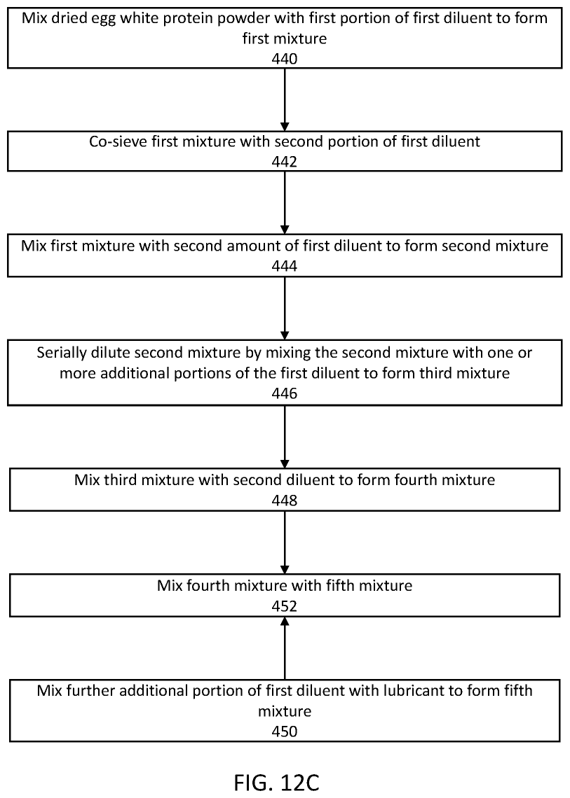

An exemplary method of manufacturing a low-dose formulation of egg white protein is depicted in FIG. 12A. In step 402, dried egg white protein powder (which is preferably characterized to ensure total protein levels and specific allergen levels) is mixed with a first amount of a first diluent (such as pregelatinized starch) to form a first mixture. In some embodiments, step 402 further comprises characterizing the dried egg white protein powder before mixing with a first amount of a first diluent. In step 404, the first mixture is co-sieved with a second amount of the first diluent. In step 406 the co-sieved first mixture and second amount of the first diluent are mixed to form a second mixture. Step 408 allows additional amounts of the first diluent to be added in a step-wise manner to the second mixture. Step 408 may comprise one, two, three, or more sub-steps of adding additional amounts of the first diluent and mixing. Performing these steps in serial allows for greater content uniformity. In step 410, the second mixture is mixed with a second diluent (such as microcrystalline cellulose) to form a third mixture. In step 412, the third mixture is mixed by high-shear mixing (such as by a conical mill) to disperse particle aggregates. In step 414, a portion of the dispersed third mixture is mixed with a lubricant to form a fourth mixture. Step 414 may also comprise mixing the portion of the dispersed third mixture with an additional amount of the first diluent. In step 416, the fourth mixture is passed through a mesh screen. In step 418 the screened fourth mixture and an additional portion of the dispersed third mixture are combined and then subsequently mixed in step 420 to form the egg white protein formulation. A batch of egg white protein formulation produced using these methods can be assayed for blend uniformity or quality characteristics as further described herein. In some embodiments, the methods further comprise assaying the blend uniformity and/or quality characteristics of the egg white protein formulation.

In another exemplary method of making an egg white protein formulation, the method can include (a) mixing dried egg white protein powder with a first amount of a first diluent (such as pregelatinized starch) to form a first mixture; (b) co-sieving the first mixture with a second portion of the first diluent through a mesh screen; (c) mixing the first mixture with the second portion of the first diluent to form a second mixture; (d) mixing the second mixture with a third portion of the first diluent to form a third mixture; (e) mixing the third mixture with a fourth portion of the first diluent to form a fourth mixture; (f) mixing the fourth mixture with a fifth portion of the first diluent to form a fifth mixture; (g) mixing the fifth mixture with a second diluent (such as microcrystalline cellulose) to form a sixth mixture; (h) mixing the sixth mixture, optionally using a higher shear force than the shear force used to mix the fifth mixture with the second diluent in step (g); (i) mixing a lubricant (such as magnesium stearate) and a sixth portion of the first diluent to form a seventh mixture, and optionally passing the seventh mixture through a mesh screen; and (k) mixing the seventh mixture with the sixth mixture to form the egg white protein formulation. This method of manufacturing the formulation is particularly useful for formulations used to manufacture lower-dose dosage containers containing the formulation, such as doses of about 0.1 mg to about 12 mg (such as about 0.2 mg, about 1 mg, about 3 mg, about 6 mg, or about 12 mg dosage containers, or any dosage therebetween). The formulation may have, for example, about 0.05 wt % to about 2.5% of egg white protein. In some embodiments, the formulation is free or substantially free of a glidant. In some embodiments, the formulation is free or substantially free of colloidal silicon dioxide). In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

An exemplary method of manufacturing a low-dose formulation of egg white protein is depicted in FIG. 12B. At step 422, dried egg white protein powder is mixed with a first portion of a first diluent (such as pregelatinized starch) to form a first mixture. At step 424, the first mixture is co-sieved through a mesh screen with a second portion of the first diluent, and the combined first mixture and second portion of the first diluent is mixed at step 426 to form a second mixture. At step 428, the second mixture is mixed (for example, using a blender, such as a tumble blender) with a third portion of the first diluent to form a third mixture. At step 430, the third mixture is mixed (for example using a blender, such as a tumble blender) with a fourth portion of the first diluent to form a fourth mixture. At step 432, the fourth mixture is mixed (for example, using a blender, such as a tumble blender) with a fifth portion of the first diluent to form a fifth mixture. At step 434, the fifth mixture is mixed with a second diluent (such as microcrystalline cellulose) to form a sixth mixture. Optionally, the sixth mixture is mixed using a higher shear force than used at step 434, for example using a conical mill. At step 436, a sixth portion of the first diluent is mixed with a lubricant (such as magnesium stearate) to form a seventh mixture, which is mixed with the sixth mixture at step 438 to form an eight mixture (e.g., the formulated composition.

In another exemplary method of making an egg white protein formulation, the method can include (a) mixing dried egg white protein powder with a first amount of a first diluent (such as pregelatinized starch) to form a first mixture; (b) co-sieving the first mixture with a second portion of the first diluent through a mesh screen, and optionally mixing the co-sieved composition; (c) serially diluting the co-sieved composition using one or more additional portions (e.g., 1, 2, 3, 4, 5 or more additional portions) of the first diluent, and mixing the composition after adding each portion to form a third mixture; (d) mixing the third mixture with a second diluent (such as microcrystalline cellulose) to form a fourth mixture; (e) mixing a further additional portion of the first diluent with a lubricant (such as magnesium stearate) to form a fifth mixture; and (f) mixing the fourth mixture with the fifth mixture to form the formulated composition. This method of manufacturing the formulation is particularly useful for formulations used to manufacture lower-dose dosage containers containing the formulation, such as doses of about 0.1 mg to about 12 mg (such as about 0.2 mg, about 1 mg, about 3 mg, about 6 mg, or about 12 mg dosage containers, or any dosage therebetween). The formulation may have, for example, about 0.05 wt % to about 2.5% of egg white protein. In some embodiments, the formulation is free or substantially free of a glidant. In some embodiments, the formulation is free or substantially free of colloidal silicon dioxide). In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

An exemplary method of manufacturing a low-dose formulation of egg white protein is depicted in FIG. 12C. A step 440, dried egg white protein powder is mixed with a first portion of a first diluent (such as pregelatinized starch) to form a first mixture. At step 442, the first mixture is co-sieved through a mesh screen with a second portion of the first diluent. At step 444, the first mixture is mixed with the second amount of the first diluent to form a second mixture. At step 446, the second mixture is serially diluted by mixing the second mixture with one or more additional portions of the first diluent to form the third mixture. The serially dilution may be performed, for example, in 1, 2, 3, 4, 5, or more sub-steps, wherein a portion of the first diluent is mixed with the composition at each sub-step before adding an additional portion of the first diluent. At step 448, the third mixture is mixed with a second diluent (such as microcrystalline cellulose) to form a fourth mixture. At step 450, another portion of the first diluent is mixed with a lubricant (such as magnesium stearate) to form a fifth mixture, which is mixed with the fourth mixture at step 452 to form the formulated composition.

The egg white protein formulation can then be packaged in a packaging, for example in a capsule or sachet, to obtain the desired dosage amount of egg white protein in a dosage container. About 100 mg to about 1000 mg (such as about 100 mg to about 250 mg, about 250 mg to about 400 mg, about 400 mg to about 600 mg, or about 600 mg to about 1000 mg) of the egg white protein formulation can be included in a dosage container. By way of example, in some embodiments about 180 mg of egg white protein formulation is included in a container (such as a capsule) to obtain the desired amount of egg white protein formulation in the dosage container.

Once the egg white protein formulation is packaged in dosage containers, the dosage containers can be assayed for content uniformity.

### Medium-Dose Manufacturing Processes

Blend uniformity and content uniformity are balanced with flowability of the egg white protein formulation when manufacturing the formulation for certain doses (such as about 3 mg to about 300 mg in a dosage container, although larger doses can be produced using a similar methodology). The formulation manufacturing process generally produces a formulation having about 1.5 wt % to about 60 wt % of egg white protein (such as between about 1.5 wt % to about 3 wt %, about 3 wt % to about 8 wt %, about 8 wt % to about 15 wt %, about 15 wt % to about 30 wt %, or about 30 wt % to about 60 wt % egg white protein).

The manufactured egg white protein formulation generally includes two diluents (for example pregelatinized starch and microcrystalline cellulose), a lubricant, and optionally a glidant (such as colloidal silicon dioxide). However, for some medium-strength dosage forms, the colloidal silicon dioxide is not included in the formulation.

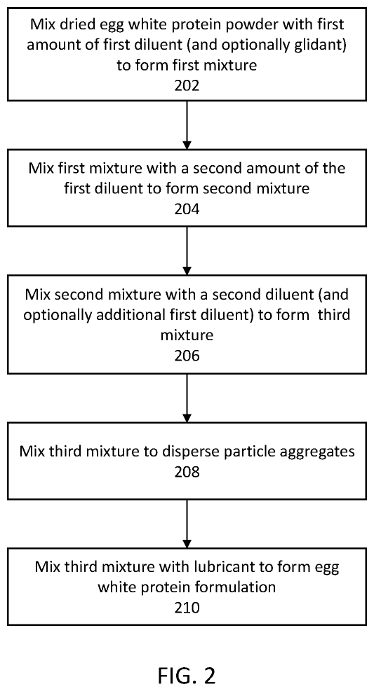

As shown in FIG. 2, dried egg white protein powder is mixed with a first amount of a first diluent (e.g., pregelatinized starch) at step 202 to form a first mixture. Optionally, a glidant (such as colloidal silicon dioxide), can also be mixed with the dried egg white protein powder at this step. Mixing is preferably done using higher-shear forces to disperse particle aggregates. This can be done, for example, using a conical mill mixer (which may be configured with a round mill impeller or a square mill impeller).

The first mixture can be further diluted by mixing the first mixture with an additional amount of the first diluent to form a second mixture, as shown at step 204 of FIG. 2. The first mixture and the additional amount of the first diluent can be mixed by shaking the combined components, or by using an impeller, a blender (such as a tumble blender), or any other suitable device.

The second mixture can be mixed with a second diluent (for example, microcrystalline cellulose) to form a third mixture, as shown in step 206 of FIG. 2. Optionally, an additional amount of the first diluent is added to the second mixture or the third mixture. The additional amount of the first diluent may be mixed with the second mixture prior to mixing with the second diluent. Alternatively, the additional amount of the first diluent may be co-mixed with the second mixture and the second diluent. The second mixture and the second diluent (and optionally the further amount of the first diluent) can be mixed by shaking the combined components, or by using an impeller, a blender (such as a tumble blender), or any other suitable device.

The third mixture is also subjected to mixing at a higher shear force than used to form the second mixtures, as shown in step 208, which can disperse particle aggregates in the mixture. The higher-shear force mixing may occur after an initial mixing of the second mixture with the second diluent, or can be the mixing force for the mixing of the second mixture with the second diluent. This can be done, for example, using a conical mill (which may be equipped, for example, using a round mill impeller or a square mill impeller) or other suitable device. In some embodiments, the second mixture and the second diluent are mixed using the higher shear force mixing to initially form the third mixture, and in some embodiments the second mixture and the second diluent are pre-mixed to form the third mixture before the third mixture is subjected to higher-shear force mixing. The higher-shear force mixing increases uniform dispersion of the dried egg white protein powder in the formulation and disperses soft, low-density agglomerates that might form. The use of the higher shear force mixing is not intended to reduce the primary particle size of any mixture components, but only to dissociate agglomerates of particles.

A lubricant (for example, magnesium stearate) is added to the mixture to form the egg white protein formulation, as shown in step 210. The lubricant can be added to any of the mixture during the manufacturing process, but is at some point mixed with the mixture containing the dried egg white protein powder, the first diluent and the second diluent. For example, the lubricant can be mixed with the third mixture before, after, or during the higher-shear force mixing step. A further amount of one or more of the diluents (e.g., the first diluent, such as pregelatinized starch) may be co-added to the mixture with the lubricant. In some embodiments, the third mixture may be mixed with the lubricant (and optionally an additional amount of the first diluent) and then mixed with an additional amount of the third mixture. In an exemplary embodiment, the third mixture, an additional amount of the first diluent, and the lubricant are mixed, passed through a mesh screen, and then mixed with an additional amount of the third mixture to form the egg white protein formulation.

Optionally, the egg white protein formulation can be passed through a mesh screen. The size of the mesh screen is generally about 250 μm to about 850 μm (such as about 300 μm to about 710 μm, or about 425 μm to about 600 μm).

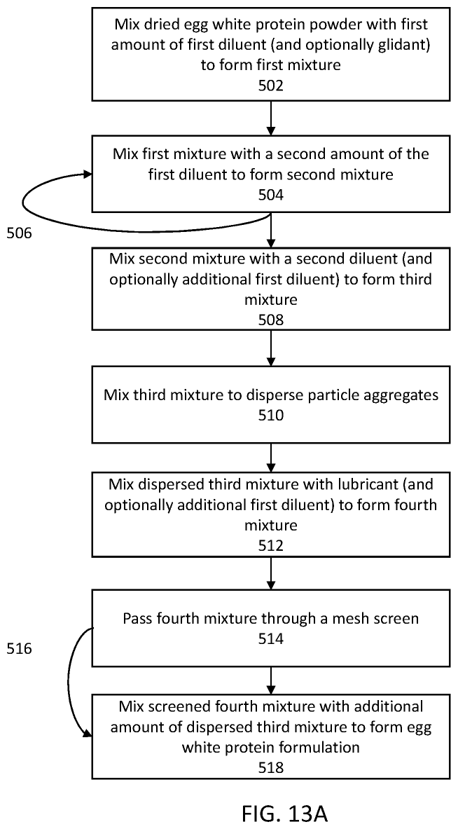

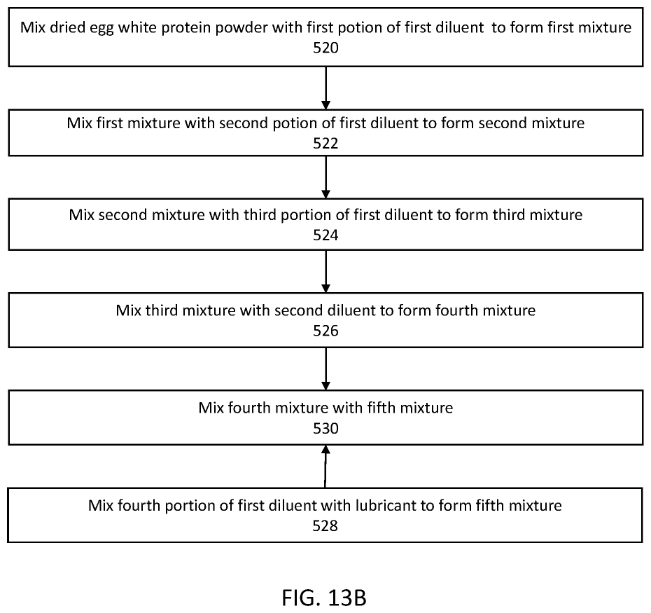

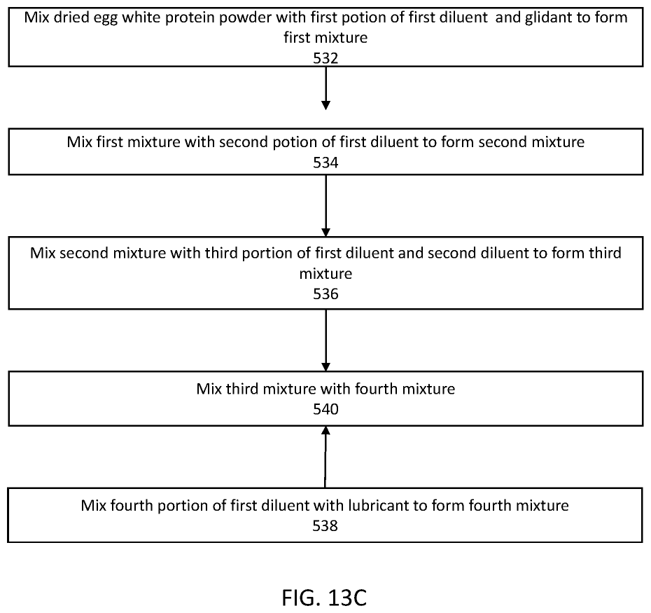

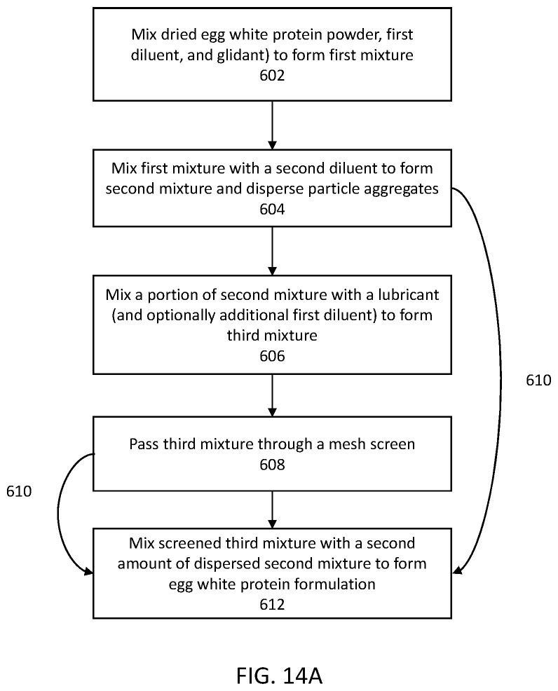

An exemplary method of manufacturing a medium-dose formulation of egg white protein is depicted in FIG. 13A. In step 502, dried egg white protein powder (which is preferably characterized to ensure total protein levels and specific allergen levels) is mixed with a first amount of a first diluent (such as pregelatinized starch) to form a first mixture. Step 502 may also comprise mixing with a glidant (such as colloidal silicon dioxide) to improve flowability. In some embodiments, step 502 further comprises characterizing the dried egg white protein powder before mixing with a first amount of a first diluent. In step 504, the first mixture is mixed with a second amount of the first diluent to form a second mixture. The mixing step of 504 may comprise two sub-steps, wherein one sub-step is at a higher shear force than the other sub-step. In an exemplary embodiment of the method, step 504 comprises two sub-steps, wherein the first sub-step is at a higher shear force than the second sub-step (such as with a conical mill), and the second sub-step is at a lower shear force (such as with a tumble blender). Step 506 is optional and allows for additional amounts of the first diluent to be added. In an exemplary embodiment, step 506 comprises mixing the second mixture with an additional amount of the first diluent. In another exemplary embodiment, step 506 is skipped. In step 508, the second mixture is mixed with a second diluent (such as microcrystalline cellulose) to form a third mixture. Step 508 may also comprise mixing the second mixture with an additional amount of the first diluent. In step 510, the third mixture is mixed with a high shear mixing step (such as a conical mill) to disperse particle aggregates. In step 512, a portion of the dispersed third mixture is mixed with a lubricant (such as magnesium stearate) to form a fourth mixture. Step 512 optionally comprises mixing the third mixture with an additional amount of the first diluent. In step 514, the fourth mixture is passed through a mesh screen. In step 516, the screened fourth mixture is combined with an additional amount of the dispersed third mixture and then subsequently mixed in step 518 to form the egg white protein formulation.

A batch of egg white protein formulation produced using these methods can be assayed for blend uniformity or quality characteristics as further described herein. In some embodiments, the methods further comprise assaying the blend uniformity and/or quality characteristics of the egg white protein formulation.

In some methods of making an egg white protein formulation, the method includes (a) mixing dried egg white protein powder with a first amount of a first diluent to form a first mixture; (b) mixing the first mixture with a second portion of the first diluent to form a second mixture; (c) mixing the second mixture with a third portion of the first diluent to form a third mixture; (d) mixing the third mixture with a second diluent to form a fourth mixture; (e) mixing the fourth mixture with a fourth portion of the first diluent and a lubricant to form a fifth mixture; (f) passing the fifth mixture through a mesh screen; (g) mixing the screened mixture of (f) with an additional amount of the fourth mixture to form the egg white protein formulation. In step (b), the mixing step may comprise two sub-steps, wherein one sub-step is at a higher shear force than the other sub-step. In an exemplary embodiment, step (b) comprises two sub-steps, wherein the first sub-step comprises mixing with a higher shear force than the second sub-step (such as with a conical mill) and the second sub-step comprises mixing with a blender (such as with a tumble blender). In step (d) the mixing step may comprise two sub-steps, wherein one sub-step is at a higher shear force than the other sub-step. In an exemplary embodiment, step (d) comprises two sub-steps, wherein the first sub-step comprises mixing with a lower shear force than the second sub-step. In some embodiments, the formulation is free or substantially free of a glidant. In some embodiments, the formulation is free or substantially free of colloidal silicon dioxide. In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

In another method of making an egg white protein formulation, the method includes (a) mixing dried egg white protein powder with a first amount of a first diluent (such as pregelatinized starch) to form a first mixture; (b) mixing the first mixture with a second portion of the first diluent to form a second mixture, and optionally mixing the second mixture at a lower shear force than the shear force used to mix the first mixture with the second portion of the first diluent; (c) mixing the second mixture with a third portion of the first diluent to form a third mixture; (d) mixing the third mixture with a second diluent (such as microcrystalline cellulose) to form a fourth mixture, and optionally mixing the fourth mixture using a higher shear force than the shear force used to mix the third mixture with the second diluent; (e) mixing a fourth portion of the first diluent with a lubricant (such as magnesium stearate) to form a fifth mixture, and optionally passing the fifth mixture through a mesh screen; and (f) mixing the fourth mixture with the fifth mixture. In some embodiments, the formulation is free or substantially free of a glidant. In some embodiments, the formulation is free or substantially free of colloidal silicon dioxide. In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

FIG. 13B shows another exemplary method of making an egg white protein formulation. Step 520 includes mixing dried egg white protein powder with a first portion of a first diluent (such as pregelatinized starch) to form a first mixture. At step 522, the first mixture is mixed with a second portion of the first diluent to form a second mixture. This step may include two or more sub-steps with different mixing shear forces. For example, the first mixture may be mixed with a second portion of the first diluent using a first mixing shear force (for example, using a conical mill) to form the second mixture, and the second mixture may be further mixed using a second shear force lower than the first shear force (for example, using a blender, such as a tumble blender). At step 524, the second mixture is mixed with a third portion of the first diluent to form a third mixture. At step 526, the third mixture is mixed with a second diluent (such as microcrystalline cellulose) to form a fourth mixture. This step may include two or more sub-steps with different mixing shear forces. For example, the third mixture may be mixed with the second diluent using a first mixing shear force (for example, using a blender, such as a tumble blender) to form the fourth mixture, and the fourth mixture may be further mixed using a second shear force higher than the first shear force (for example, using a conical mill). At step 528, a fourth portion of the first diluent is mixed with a lubricant (such as magnesium stearate) to form a fifth mixture, which is optionally passed through a mesh screen. At step 530, the fourth mixture is mixed with the fifth mixture to form the egg white protein formulation.

In another method of making an egg white protein formulation, the method includes (a) mixing dried egg white protein powder with a first amount of a first diluent (such as pregelatinized starch) and a glidant (such as colloidal silicon dioxide) to form a first mixture; (b) mixing the first mixture with a second portion of the first diluent to form a second mixture, and optionally mixing the second mixture at a lower shear force than the shear force used to mix the first mixture with the second portion of the first diluent; (c) mixing the second mixture with a third portion of the first diluent to form a third mixture; (d) mixing the third mixture with a second diluent (such as microcrystalline cellulose) and a fourth portion of the first diluent to form a fourth mixture, and optionally mixing the fourth mixture using a higher shear force than the shear force used to mix the third mixture with the second diluent; (e) mixing a fifth portion of the first diluent with a lubricant (such as magnesium stearate) to form a fifth mixture, and optionally passing the fifth mixture through a mesh screen; and (f) mixing the fourth mixture with the fifth mixture. In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

FIG. 13C shows another exemplary method of making an egg white protein formulation. Step 532 includes mixing dried egg white protein powder with a first portion of a first diluent (such as pregelatinized starch) and a glidant (such as colloidal silicon dioxide) to form a first mixture. At step 534, the first mixture is mixed with a second portion of the first diluent to form a second mixture. This step may include two or more sub-steps with different mixing shear forces. For example, the first mixture may be mixed with a second portion of the first diluent using a first mixing shear force (for example, using a conical mill) to form the second mixture, and the second mixture may be further mixed using a second shear force lower than the first shear force (for example, using a blender, such as a tumble blender). At step 536, the second mixture is mixed with a third portion of the first diluent and a second diluent (such as microcrystalline cellulose) to form a third mixture. This step may include two or more sub-steps with different mixing shear forces. For example, the third mixture may be mixed with the second diluent using a first mixing shear force (for example, using a blender, such as a tumble blender) to form the fourth mixture, and the fourth mixture may be further mixed using a second shear force higher than the first shear force (for example, using a conical mill) At step 538, a fourth portion of the first diluent is mixed with a lubricant (such as magnesium stearate) to form a fifth mixture, which is optionally passed through a mesh screen. At step 540, the fifth mixture is mixed with the fourth mixture.

The egg white protein formulation can then be packaged in a packaging, for example in a capsule or sachet, to obtain the desired dosage amount of egg white protein in a dosage container. About 100 mg to about 1000 mg (such as about 100 mg to about 250 mg, about 250 mg to about 400 mg, about 400 mg to about 600 mg, or about 600 mg to about 1000 mg) of the egg white protein formulation can be included in a dosage container. By way of example, in some embodiments about 180 mg of egg white protein formulation is included in a container (such as a capsule) to obtain the desired amount of egg white protein formulation in the dosage container. In some embodiments about 500 mg of egg white protein formulation is included in a container (such as a capsule) to obtain the desired amount of egg white protein formulation in the dosage container.

Once the egg white protein formulation is packaged in dosage containers, the dosage containers can be assayed for content uniformity.

### High-Dose Manufacturing Processes

The manufacture of egg white protein formulation for use in certain strength doses (such as about 100 mg or more) is designed to prioritize flowability of the formulation. The egg white protein powder can stick to packaging material, limiting the deliverability of the formulation from the container. To increase the flowability, the formulation is generally manufactured with a glidant (such as colloidal silicon dioxide) in addition to one or more diluents (such as pregelatinized starch and/or microcrystalline cellulose) and a lubricant (such as magnesium stearate). In some embodiments, the egg white protein formulation has about 50 wt % to about 80 wt % of egg white protein (such as about 50 wt % to about 60 wt %, about 60 wt % to about 70 wt %, or about 70 wt % to about 80 wt %).

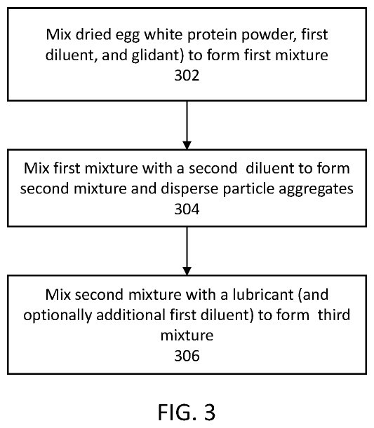

To manufacture the egg white protein formulation, dried egg white protein powder, a first diluent (such as pregelatinized starch), and a glidant (such as colloidal silicon dioxide) are mixed together, as shown at step 302 of FIG. 3. The components can be mixed by shaking the combined components, or by using an impeller, a blender (such as a tumble blender), or any other suitable device.

The first mixture is then mixed with a second diluent (such as microcrystalline cellulose) to form a second mixture, as shown in step 304. The first mixture and the second diluent can be mixed using a higher shear force than used to mix the egg white protein powder with the first diluent and the glidant. The higher-shear force can dissociate particle agglomerates within the mixture.

The second mixture also mixed with a lubricant to form the egg white protein formulation, as shown in step 306. Optionally, an additional amount of the first diluent can also be mixed with the second mixture, before, after, or at the same time that the lubricant is mixed with the second mixture. In some embodiments, the second mixture may be mixed with the lubricant (and optionally an additional amount of the first diluent) and then mixed with an additional amount of the second mixture. In an exemplary embodiment, the second mixture, an additional amount of the first diluent, and the lubricant are mixed, passed through a mesh screen, and then mixed with an additional amount of the second mixture to form the egg white protein formulation.

Optionally, the egg white protein formulation can be passed through a mesh screen. The size of the mesh screen is generally about 250 μm to about 850 μm (such as about 300 μm to about 710 μm, or about 425 μm to about 600 μm).

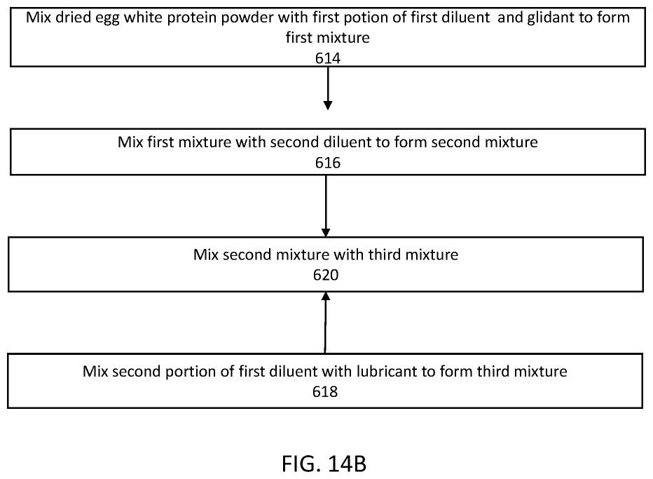

An exemplary method of manufacturing a high-dose formulation of egg white protein is depicted in FIG. 14A. In step 602, dried egg white protein powder (which is preferably characterized to ensure total protein levels and specific allergen levels) is mixed with a first amount of a first diluent (such as pregelatinized starch) and a glidant (such as colloidal silicon dioxide) to form a first mixture. In some embodiments, step 602 further comprises characterizing the dried egg white protein powder before mixing with a first amount of a first diluent. In step 604, the first mixture is then mixed with a second diluent to form a second mixture and then mixed with a high-shear mixing step (such as a conical mill) to disperse particle aggregates. In step 606, a portion of the dispersed second mixture is mixed with a lubricant to form a third mixture. Step 606 may also comprise mixing the second mixture with an additional amount of the first diluent. In step 608, the third mixture is passed through a mesh screen. In step 610, the screened third mixture and a second amount of the dispersed second mixture are combined and subsequently mixed in step 612 to form the egg white protein formulation.

In some methods of making an egg white protein formulation, the method includes (a) mixing dried egg white protein powder with a first amount of a first diluent (such as pregelatinized starch) to form a first mixture; (b) mixing the first mixture with a second diluent (such as microcrystalline cellulose), optionally at a higher shear force than used to form the first mixture in step (a); (c) mixing a second portion of the first diluent with a lubricant (such as magnesium stearate) to form a third mixture, and optionally passing the third mixture through a mesh screen; and (d) mixing the second mixture with the third mixture. In some embodiments the dried egg white protein powder (i.e., before being formulated) and/or egg white protein formulation produced using these methods (i.e., after being formulated) can be assayed for blend uniformity or quality characteristics as further described herein, such as using an HPLC assay (e.g., a RP-HPLC) or ELISA assay to determine the concentration or profile of one or more of ovalbumin, ovomucoid, lysozyme, and/or ovotransferrin, or the total protein content of the composition. The formulated composition may be packaged, for example in a capsule or sachet.

Another exemplary method of manufacturing a high-dose formulation of egg white protein is depicted in FIG. 14B. At step 614, dried egg white protein powder is mixed with a first portion of a first diluent (such as pregelatinized starch) with a glidant (such as colloidal silicon dioxide) to form a first mixture. At step 616, the first mixture is mixed with a second diluent (such as microcrystalline cellulose) to form a second mixture. In some embodiments, the first mixture is mixed with the second diluent at a higher shear force (for example, using a conical mill) that the dried egg white protein powder was mixed with the first portion of the first diluent and the glidant to form the first mixture at step 614, which may have been mixed, for example, using a blender, such as a tumble blender. At step 618, a second portion of the first diluent is mixed with a lubricant (such as magnesium stearate) to form a third mixture, which is optionally passed through a mesh screen. At step 620, the second mixture is mixed with the third mixture.

A batch of egg white protein formulation produced using these methods can be assayed for blend uniformity or quality characteristics as further described herein. In some embodiments, the methods further comprise assaying the blend uniformity and/or quality characteristics of the egg white protein formulation.

The egg white protein formulation can then be packaged in a packaging, for example in a capsule or sachet, to obtain the desired dosage amount of egg white protein in a dosage container. About 100 mg to about 1000 mg (such as about 100 mg to about 250 mg, about 250 mg to about 400 mg, about 400 mg to about 600 mg, or about 600 mg to about 1000 mg) of the egg white protein formulation can be included in a dosage container. By way of example, in some embodiments about 500 mg of egg white protein formulation is included in a container (such as a capsule) to obtain the desired amount of egg white protein formulation in the dosage container.

Once the egg white protein formulation is packaged in dosage containers, the dosage containers can be assayed for content uniformity.

### Quality Control Processes

The dosage forms and egg white protein formulations described herein can be used for oral immunotherapy, wherein increasing doses are administered to a patient allergic to one or more egg proteins to desensitize the patient to those proteins. Because an allergic response to allergenic proteins can be severe and even life threatening, it is desirable that the administered doses be adequately controlled both quantitatively and qualitatively. To ensure quality control of the product administered to a patient, various parameters of the dried egg white protein powder, egg white protein formulation, and dosage containers (and dosage container lots) containing the egg white protein formulation can be monitored. Such quality control processes can include characterizing one or allergenic more egg white proteins (ovomucoid, ovalbumin, ovotransferrin, and/or lysozyme), determining a protein content, determining a blend uniformity of the egg white protein formulation, determining a content uniformity or deliverable mass of a lot of dosage containers containing the egg white protein formulation, or determining a water activity of the dried egg white protein powder or the egg white protein formulation. The quality control methods can be included in a manufacturing process to ensure consistent manufacturing of the egg white protein formulation or dosage containers.

Allergenic proteins in egg white include Gal d 1 (ovomucoid), Gal d 2 (ovalbumin), Gal d 3 (ovotransferrin), and Gal d 4 (lysozyme). One or more (or all) of these proteins in the egg white protein formulation or the dried egg white protein powder can be characterized using the methods described herein. The characterization of the one or more allergenic egg white proteins can include determining the presence of the one or more proteins, an amount or relative amount of one or more immunodominant egg white proteins, or a potency or relative potency of one or more immunodominant egg white proteins.

One method of characterizing ovomucoid, ovalbumin, ovotransferrin and/or lysozyme includes analyzing the dried egg white protein powder or egg white protein formulation using high-performance liquid chromatography (HPLC). Exemplary HPLC techniques include size-exclusion chromatograph (SEC-HPLC) and reversed-phase HPLC (RP-HPLC). RP-HPLC, for example, adequately separates all four allergenic egg white proteins for analysis, either qualitatively or quantitatively. An HPLC profile can be obtained, and peaks representing ovomucoid, ovalbumin, ovotransferrin and/or lysozyme can be identified or compared to a reference standard for qualitative assessment.

Characterizing ovomucoid, ovalbumin, ovotransferrin and/or lysozyme can include determining the presence of one or more of the ovomucoid, the ovalbumin, the ovotransferrin and/or the lysozyme in dried egg white protein powder (the drug substance) and/or an egg white protein formulation (the drug product). This can be done, for example, by polyacrylamide gel electrophoresis (PAGE), such as SDS-PAGE, an immunoblot, or HPLC (such as SEC-HPLC or RP-HPLC). The allergenic proteins generally elute from an HPLC column at consistent time points, and the peaks can be identified using a reference standard (such as a purified commercially available ovomucoid, ovalbumin, ovotransferrin or lysozyme). The allergenic proteins in the dried egg white protein powder or egg white protein formulation can be identified using an HPLC profile, for example based on retention time. An HPLC profile can also be qualitatively compared to a reference standard to determine consistency of the tested dried egg white protein powder or egg white protein formulation.

Characterizing ovomucoid, ovalbumin, ovotransferrin and/or lysozyme can include quantifying an amount of ovomucoid, ovalbumin, ovotransferrin and/or lysozyme in the egg white protein formulation or the dried egg white protein powder. The HPLC (e.g., SEC-HPLC or RP-HPLC) methods can be used to adequately quantify the proteins, for example by determining an area of the peak in the HPLC profile. A concentration of ovomucoid, ovalbumin, ovotransferrin and/or lysozyme in the egg white protein formulation or dried egg white powder can be determined, for example, using this method by comparing the protein peak associate with the ovomucoid, ovalbumin, ovotransferrin and/or lysozyme to a reference. Another example of characterizing the proteins includes determining a relative amount of ovomucoid, ovalbumin, ovotransferrin and/or lysozyme can be determined using the HPLC profile. The relative amount could be compared to total protein in the egg white protein formulation or dried egg white protein formulation (for example by comparing to the total area of all protein peaks) or compared to the total amount of ovomucoid, ovalbumin, ovotransferrin and/or lysozyme. The HPLC profile is established by measuring light absorbance as the proteins elute from the column, such as a light wavelength of about 210 nm to about 280 nm). The peak area due to a protein in the HPLC profile correlates with the amount of that protein in the assayed sample. However, the ratio of peak areas between different proteins may not necessarily reflect the weight ratio of the different proteins due to differences in absorbance of the proteins. A weight ratio of the different proteins can be established by calibrating the peak area to mass for the investigated protein. Accordingly, relative amounts of a protein may be, for example, a relative peak area from an HPLC profile or a relative weight of the proteins. Any basis for the relative amount of the allergenic proteins may be used, as the quality control process is used to ensure consistency between lots.

One or more of ovomucoid, ovalbumin, ovotransferrin and/or lysozyme can be also be characterized to determine the potency or relative potency (relative to a potency of the protein in a reference sample) of the allergenic egg white protein in the dried egg white protein powder or the egg white protein formulation. The potency or relative potency of an allergenic protein may be altered due to denaturation of the allergenic protein, and measuring the potency or relative potency of the allergenic protein in a dried egg white protein powder or an egg white protein formulation can be useful to monitor quality or consistency of the powder and/or formulation. In some embodiments, the potency of one or more of the egg white proteins in the dried egg white protein powder or the egg white protein formulation is measured in vitro. In some embodiments, a single allergenic protein (e.g., ovomucoid) is used to represent the potency or relative potency of the powder or formulation. The potency may be reported, for example, as an EC₅₀ or a ratio of EC₅₀s (i.e., a relative potency) measured by a potency assay. Exemplary immunoassay techniques that can be used to determine potency of one or more allergenic egg white proteins include an enzyme-linked immunosorbent assay (ELISA), a radioimmunoassay (RIA), an immunoblot, surface plasmon resonance (SPR), or a multiplexed immunoassay. Another method of measuring potency of the ovalbumin, ovotransferrin, ovomucoid, or lysozyme in the egg white protein formulation can include a cell-based assay (e.g., a basophil histamine release assay). See, for example, Santos et al., Basophil activation test: food challenge in a test tube or specialist research tool?, Clinical and Translational Allergy, vol. 6, no. 10, pp. 1-9 (2016).

The reporter molecule used in the potency assay binds the assayed allergenic protein with high specificity, and is generally an antibody. The antibody may be a monoclonal antibody or a polyclonal antibody. In some embodiments, the antibody is an IgG antibody or an IgE antibody. The antibody used to determine potency or relative potency specifically binds the assayed protein (e.g., ovalbumin, ovotransferrin, ovomucoid, or lysozyme). An antibody pool that includes antibodies that specifically bind two or more different proteins can also be used to simultaneously determine the potency or relative potency of two or more assay protein. For example, the pool can include two or more of an antibody that specifically binds ovalbumin, an antibody that specifically binds ovotransferrin, an antibody that specifically binds ovomucoid, and/or an antibody that specifically binds lysozyme. The pool of antibodies can be a pool of separately purified antibodies that are mixed together, or can be derived from an animal immunized with whole egg proteins or egg white proteins. The same antibody or pool of antibodies should be used for the test and reference samples when measuring the relative potency.

A protein content of the dried egg white protein powder or the egg white protein formulation, either before or after packaging in a dosage form, can be determined to monitor quality during the manufacturing process. The protein content can be reported as a concentration (e.g., a weight percentage of a composition) or in reference to a target (i.e., intended) amount of protein (e.g., X % of a target dose). The protein content of the dried egg white protein powder is generally measured to determine how much dried egg white protein powder should be included in the manufacturing process to obtain an intended egg white protein concentration in the manufactured egg white protein formulation. The protein content can also be used to measure blend uniformity in an egg white protein formulation or content uniformity in a lot of manufactured dosage containers containing the egg white protein formulation. Exemplary methods of measuring protein content include light absorbance, a Lowry assay, a Bradford assay, a combustion assay, a bicinchoninic acid (BCA) assay, HPLC (such as SEC-HPLC, RP-HPLC, or any other chromatography method that can quantitatively measure an amount of protein), or any other suitable quantitative protein assay.

The deliverable mass of egg white protein formulation from a dosage container can be measured on an individual dosage container basis or as a sample of dosage containers taken from a manufactured lot. The deliverable mass for a lot of dosage containers can be determined by sampling a plurality of dosage containers selected from the lot (for example about 2, 3, 4, 5, 6, 7, 8, 9, 10 or more dosage containers). A composition is deliverable from a container when the composition can be recovered from the container by pouring the composition from the container, shaking the container, or striking the container. The deliverable mass can be determined by weighing the contents of a dosage form that are deliverable from the dosage container under normal use. A composition that can only be removed from the container by inserting a mechanical device into the container or adding a fluid (such as compressed gas or liquid) into the container is not deliverable from the container.

The water activity of the dried egg white protein powder or the egg white protein formulation can be measured. Water activity can be measured by use of a water activity meter, such as an AQUALAB 4TEV. The dried egg protein formulation should be relatively dry to avoid microbial growth and/or degradation of the allergenic egg white proteins.

### Oral Immunotherapy Methods

The patients treated by using the oral immunotherapy methods described herein are human individuals with an egg allergy (preferably a hen egg allergy). The egg allergy may be an allergy to raw egg white proteins, or an allergy to cooked egg proteins (either cooked egg white proteins or cooked whole egg proteins). The cooked egg protein may be baked.

The egg white protein formulations and dosage forms described herein can be used to treat human patients allergic to egg (preferably a hen egg) using oral immunotherapy. The egg allergy may be an allergy to raw egg white proteins, or an allergy to cooked egg proteins (either cooked egg white proteins or cooked whole egg proteins, such as baked egg white proteins or baked whole egg proteins). The most abundant allergenic proteins found in egg white are ovalbumin, ovomucoid, ovotransferrin, and lysozyme, and the patient may be allergic to one or more of these allergenic proteins.

Methods for diagnosing an egg allergy are known in the art. For example, the patient may be diagnosed with a skin-prick test (SPT), an egg-white protein specific IgE (ew-IgE) level, an oral food challenge, or an association of one or more allergic responses with egg protein consumption. For example, in some embodiments, the patient has a serum ew-IgE level of about 0.35 kU_{A}/L or more, about 0.7 kU_{A}/L, about 3.5 kU_{A}/L or more, about 5 kU_{A}/L or more, or about 7 kU_{A}/L or more. The serum ew-IgE level can be determined using a quantitative immunoassay. Quantitative immunoassays are known in the art, and can include, but are not limited to, an enzyme-linked immunosorbent assay (ELISA); an alkaline phosphatase immunoassay auto-analyzer, such as an IMMULITE® system (Siemens Healthcare Diagnostics, Erlangen, Germany); a radioallergosorbent test (RAST), or a fluoroenzyme immunoassay auto-analyzer, such as the ImmunoCAP® system (Thermo Fisher Scientific/Phadia, Uppsala, Sweden). The fluoroenzyme immunoassay auto-analyzer is a preferred method.

The patient is generally about 4 years of age or older, such as between about 4 years of age and 26 years of age.

A baseline highest tolerated dose or baseline cumulative tolerated dose of raw egg white protein or cooked egg protein (either cooked egg white protein or cooked whole egg protein, such as baked egg white protein or baked whole egg protein) can be determined for the patient prior to the start of treatment through an oral food challenge. An oral food challenge involves the oral administration of a series of doses of an allergenic composition (e.g., egg white protein or cooked egg protein, such as baked egg white protein or baked whole egg protein) until a moderate or severe adverse reaction related to the administered egg white protein is elicited. Each administered dose is spaced by a period of time (e.g., about 20 minutes to about 30 minutes) and the patient is monitored for an adverse event. An exemplary food challenge is a double blind, placebo controlled food challenge (DBPCFC) described in Sampson et al., Standardizing double blind, placebo controlled oral food challenges: American Academy of Allergy, Asthma & Immunology European Academy of Allergy and Clinical Immunology PRACTALL consensus report, J. Allergy Clin. Immunol., vol. 130, no. 6, pp. 1260-74 (2012).

An oral food challenge for raw egg white protein to determine a highest tolerated dose or cumulative tolerated dose of raw egg white protein can include oral administration of a series of escalating amounts of raw egg white protein. The doses can range, for example between about 1 mg and about 2000 mg, or between about 1 mg to about 300 mg. The patient need not ingest all doses of the oral food challenge, as the oral food challenge is generally terminated after the patient suffers a moderate or severe allergenic adverse event. An exemplary set of raw egg white doses for an oral food challenge is shown in Table 1. For the exemplary oral food challenge doses shown in Table 1, if the highest dose of egg white protein tolerated by a patient is 30 mg (the highest tolerated dose), the cumulative tolerated dose is 44 mg of egg white protein.

TABLE 1
Raw egg white protein doses for oral food challenge

| Raw Egg White Protein Dose (mg) | Cumulative Dose (mg) |
|---|---|
| 1 | 1 |
| 3 | 4 |
| 10 | 14 |
| 30 | 44 |
| 100 | 144 |
| 300 | 444 |
| 600 | 1044 |
| 1000 | 2044 |
| 2000 | 4044 |

In some embodiments, the highest dose of raw egg white protein tolerated by a patient (i.e., the highest tolerated dose) at the start of treatment is about 2000 mg or less, about 1000 mg or less, about 600 mg or less, about 300 mg or less, about 100 mg or less, about 30 mg or less, about 10 mg or less, or about 3 mg or less. In some embodiments, the highest cumulative dose of raw egg white protein tolerated by a patient (i.e., the highest tolerated dose) at the start of treatment is about 444 mg or less, about 144 mg or less, about 44 mg or less, about 14 mg or less, or about 4 mg or less. As some patients are highly sensitive to raw egg white protein, to lower the risk of a severe adverse event during the course of treatment, in some embodiments the highest tolerated dose is about 0.2 mg of raw egg white protein or more, about 0.4 mg of raw egg white protein or more, about 0.8 mg of raw egg white protein or more, about 1.0 mg of raw egg white protein or more, about 1.2 mg of raw egg white protein or more, or about 2 mg of raw egg white protein or more. In some embodiments the cumulative tolerated dose is about 0.2 mg of raw egg white protein or more, about 0.6 mg of raw egg white protein or more, about 1.4 mg of raw egg white protein or more, about 2.4 mg of raw egg white protein or more, about 2.6 mg of raw egg white protein or more, about 4.4 mg of raw egg white protein or more, or about 4.6 mg of raw egg white protein or more.

An oral food challenge for cooked egg protein or baked egg protein can also be used to determine a highest tolerated dose for cooked or baked egg protein. The cooked or baked egg protein may be from cooked or baked egg white, or cooked or baked whole egg (i.e., egg white and yolk). For an oral food challenge, a series of doses of cooked egg protein (which may be, for example, cooked in a baked food product, such as bread, a muffin, a cookie, or a cake; or fried). An exemplary set of cooked or baked egg doses for an oral food challenge is shown in Table 2, which has doses of cooked whole egg protein in a baked muffin. In some embodiments, the baked egg protein or baked egg white protein is heated at about 176° C. to about 260° C. (such as about 176° C. to about 232° C., about 176° C. to about 220° C., or about 176° C. to about 205° C.) for about 20 minutes to about 60 (such as about 20 minutes to about 45 minutes, about 20 minutes to about 35 minutes, or about 20 minutes to about 30 minutes).

TABLE 2
Cooked whole egg protein doses for oral food challenge

| | Cooked/Baked Egg | Cumulative |
|---|---|---|
| Dose Number | Protein Dose (mg) | Dose (mg) |
| 1 | 125 | 125 |
| 2 | 250 | 375 |
| 3 | 500 | 875 |
| 4 | 500 | 1375 |
| 5 | 625 | 2000 |

Although some patients treated with the oral immunotherapy described herein are able to tolerate cooked egg protein, some patients are unable to tolerate any or large amounts of cooked or baked egg protein. In some embodiments, the highest dose of cooked egg protein or baked egg protein tolerated by a patient (i.e., the highest tolerated dose) at the start of treatment is about 625 mg or less, about 500 mg or less, about 250 mg or less, or about 125 mg or less. In some embodiments, the highest cumulative dose of cooked egg protein or baked egg protein tolerated by a patient (i.e., the highest tolerated dose) at the start of treatment is about 2000 mg or less, about 1375 mg or less, about 875 mg or less, about 375 mg or less, or about 125 mg or less.

### Oral Immunotherapy Treatment Schedule

A patient is treated for an egg allergy by orally administering a plurality of doses of a pharmaceutical composition comprising egg white protein to the patient according to an oral immunotherapy (OIT) schedule. The OIT schedule typically includes an up-dosing phase, which is followed by a maintenance phase. Optionally, the OIT schedule further includes an initial escalation phase, which occurs prior to the start of the up-dosing phase.

The doses can be provided in one or more dosage containers (e.g., capsules or sachets) that contain the pharmaceutical composition (also referred to herein as an “egg white protein formulation”). The dosage containers are typically not ingested whole, but are generally opened prior to administration and the pharmaceutical composition contained therein is orally consumed. The pharmaceutical composition can be mixed with a food vehicle, which is eaten by the patient. Exemplary food vehicles include applesauce, pudding, oatmeal, beverages (e.g., a shake), or any other suitable food product that mixes with the pharmaceutical composition.

### Up-Dosing Phase

The up-dosing phase of the OIT schedule includes orally administering to the patient a series of escalating daily doses of egg white protein. The egg white protein is generally raw, although it may be pasteurized or otherwise treated in a manner that does not affect protein epitope presentation. The egg white protein is contained within the egg white protein formulation, which can be manufactured, for example, according to the methods described herein. The doses of the egg white protein are preferably administered to the patient on a daily basis, although the dose may be skipped, delayed, or a portion of the dose delayed if the patient experiences one or more adverse events related to the administration of the egg white protein or a concurrent factor associated with increased sensitivity to an allergen not related to the administration of the egg white protein (such as an atopic disease flare-up, inflammation, an illness, menses, or unintended exposure to a food that the patient is allergic to).

The series of escalating doses of egg white protein generally range from about 1 mg to about 300 mg. A given dose is administered to a patient on a daily basis (unless an adjustment is made) for a period of at least two weeks before the dose is escalated to a higher dose. Escalation to a higher dose may take longer than two weeks, for example, due to an adverse event related to the egg white protein or some other concurrent factor associated with increased sensitivity to an allergen not related to the egg white protein, which justifies a delay in increasing the dose. The dose administered during the up-dosing phase is escalated only if the patient tolerates the previous dose. Therefore, in some circumstances, the same dose may be administered to the patient for more than two weeks, such as three or more weeks, or four or more weeks. To ensure the dosing is not escalated too quickly, the up-dosing phase generally lasts about 20 weeks or more, for example about 20 weeks to about 44 weeks.

The up-dosing phase may include 4, 5, 6, 7, 8, 9, or 10 or more different doses that are administered to the subject, which can range from about 1 mg to about 300 mg. The different doses are spaced within this dose range, but need not be evenly spaced. Exemplary doses that can be administered to subject can include about 1 mg, about 3 mg, about 6 mg, about 12 mg, about 20 mg, about 40 mg, about 80 mg, about 120 mg, about 160 mg, about 200 mg, about 240 mg, and about 300 mg of egg white protein. In some embodiments, the up-dosing phase includes administration of at least a 1 mg dose and a 300 mg of the egg white protein. In some embodiments, the maximum dose administered to the patient during the up-dosing phase is 300 mg of egg white protein.

An escalated dose is preferably administered in a clinical setting (e.g., a doctor's office, a hospital, or other facility that allows for an immediate medical response if a moderate or severe allergic adverse event occurs). For example, if a patient has completed a two week period of daily administration of an 80 mg dose of egg white protein, a 120 mg dose of the egg white protein may be administered in a clinical setting. If the patient tolerates the escalated dose, the following doses at that dose amount can be administered to the subject outside the clinical setting, such as self-administered by the patient at home.

### Maintenance Phase

Following the up-dosing phase, the OIT schedule includes a maintenance phase. The maintenance phase includes the administration of doses of the egg white protein to the patient for a period of time, and is intended to sustain the state of desensitization after completion of the treatment. Generally, the maintenance phase is about 12 weeks in length or more, although it need not have a definitive end point. In some embodiments, the maintenance dose is administered on a daily basis, although adjustments to the dosage administration can be made, as discussed herein.

The egg white protein dose administered during the maintenance phase (i.e., the “maintenance dose”) is usually the highest tolerated dose obtained by the patient during the up-dosing phase, although the dose may be reduced under certain circumstances. For example, if the patient successfully completes an up-dosing phase that includes administration of 300 mg of egg white protein, the maintenance dose is typically 300 mg of egg white protein, but may be lowered. For example, a maintenance dose may be reduced, skipped, delayed, or a portion delayed if the patient experiences one or more adverse events related to the administration of the egg white protein or a concurrent factor associated with increased sensitivity to an allergen not related to the administration of the egg white protein (such as an atopic disease flare-up, inflammation, an illness, or menses).

### Initial Escalation Phase

Optionally, an initial escalation phase precedes the up-dosing phase in the OIT schedule. The initial escalation phase includes orally administering to the patient as series of escalating dose of egg white protein in a single day. The doses are separated by a period of time, generally about 20 minutes or more (such as about 20 minutes to about 60 minutes, or about 20 minutes to about 30 minutes). The temporal separation allows the patient to be monitored for an adverse event related to the egg white protein before the next dose is administered. Generally, the initial escalation phase occurs in a clinical setting.

The egg white protein doses administered to the subject can be, for example about 0.2 mg of egg white protein to about 2 mg egg white protein. The initial escalation phase can include 2, 3, 4, 5, 6 or more doses. Exemplary doses administered to a subject can include about 0.2 mg, about 0.4 mg, about 0.8 mg, about 1.2 mg, and about 2 mg of egg white protein. In an additional exemplary embodiment, doses administered to a subject can include about 0.2 mg, about 0.4 mg, about 0.8 mg, about 1.0 mg, and about 2 mg of egg white protein.

### Dosage Adjustments

The oral immunotherapy schedule may be adjusted slightly if the patient experiences an adverse event related to administration of a dose of the egg white protein (i.e., an allergic response to the dose) or a concurrent factor associated with increased sensitivity to an allergen that is not related to the administration of the dose of the egg white protein. The adverse event related to the administration of the egg white protein may be a mild allergenic adverse event, a moderate allergenic adverse event, or a severe allergenic adverse event. This dosage adjustment can occur during the up-dosing phase of the OIT schedule or the maintenance phase of the OIT schedule. The dose adjustment can be a reduction of the dose, skipping a scheduled dose administration, delaying a portion of the dose, or delay escalating a dose. By adjusting the dosage schedule, the risk of experiencing a more severe adverse event (if the dosage is adjusted in response to an adverse event related to the administration of the egg white protein), or experiencing an adverse event or the severity of the adverse event (if the dosage is adjusted in response to an adverse event not related to the administration of the egg white protein) is decreased.

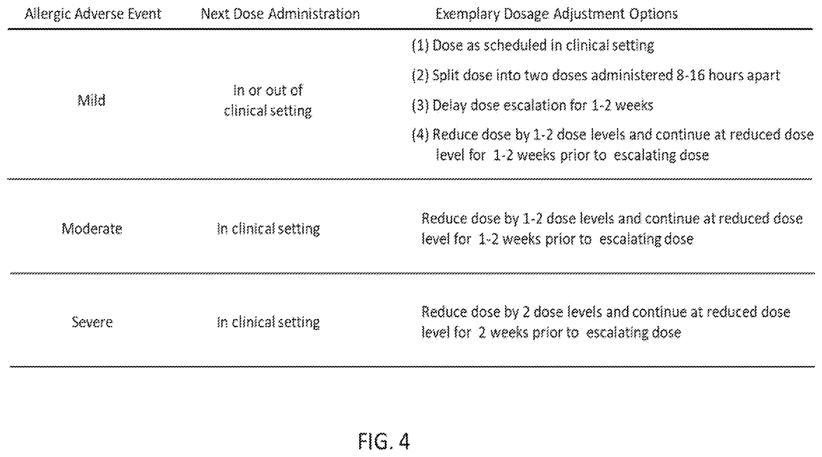

The adverse event related to the administration of the egg white protein that may result in the dosage adjustment may be, for example, a hypersensitivity, anaphylaxis (for example anaphylactic shock), or gastrointestinal symptoms (such as abdominal pain or vomiting). FIG. 4 shows an exemplary decision tree for adjusting the dosage depending on the severity of the adverse event related to the administration of the egg white protein.

The concurrent factor associated with increased sensitivity to an allergen that may result in the dosage adjustment can be any event or patient status that would enhance the sensitivity of the patient to an allergen that concurrently occurs with the administration of the pharmaceutical composition. This can be due, for example, due to an immunological change that results from the concurrent factor associated with increased sensitivity to an allergen. Exemplary concurrent factor associated with increased sensitivity to an allergens include a flare up of an atopic disease (e.g., eczema, asthma, or rhinoconjunctivitis), an allergic reaction to an allergen other than egg white protein, inflammation (for example, due to a surgery or traumatic injury), an illness (such as an infection), and menses. The concurrent factor may also be an unintended exposure (e.g., consumption) to a food that the patient is allergic to. The food that the patient is allergic to may be egg, or may be some other food that the patient is also allergic to, such as milk, peanuts, tree nuts, wheat, shellfish, or soy.

The dosage of the pharmaceutical composition can be adjusted by reducing the dose of the pharmaceutical composition. The dose reduction is temporary, and can be increased after a period of time, for example after the passing of the adverse event or concurrent factor associated with increased sensitivity to an allergen. The dose may be reduced by up to about 50%, or to the next available dose lower than 50%, relative to the previously administered dose. In some embodiments, the dose is reduced by 1 dose level or 2 dose levels below the previously administered dose. In some embodiments, one or more (such as the first) reduced dose is administered in a clinical setting. Once a dose is reduced, the reduced dose can be administered to the patient for about one week or more (such as two weeks or more, between about one week and about four weeks, or between about one week and about two weeks) prior to escalating subsequent doses. The escalated dose is preferably administered in a clinical setting so that the patient can be monitored for an adverse event related to the administration of the pharmaceutical composition. Administration of the escalated dose should be attempted, but if an allergic response results, the reduced dose can be continued to be administered to the patient.

In some embodiments, the dosage is adjusted by skipping one or more scheduled doses administration. For example, if the dose is scheduled to be administered on a daily basis, one, two, three or more daily doses may be skipped prior to resuming administration of the pharmaceutical composition. Administration of the pharmaceutical composition may be resumed at the same dose as previously administered, or at a reduced dose. If more than one dose is skipped, it is generally preferred that the next dose administered to the patient be administered in a clinical setting.

Adjusting the dosage of the pharmaceutical composition could include delaying administration of a portion of the pharmaceutical composition. For example, a first portion could be administered to the patient as scheduled, and a second portion is administered to the patient at a time prior to the next scheduled dose administration. In some embodiments, the dose is split into two approximately equal portions. The second portion may be administered to the patient about 8 hours to about 16 hours, or about 8 hours to about 12 hours, after administration of the first portion.

Dosage administration can also be adjusted during the up-dosing phase by delaying a scheduled escalation of the dose administered to the patient. The OIT schedule generally has pre-set escalation times, such as every two weeks. However, if the patient is experiencing an adverse event or a concurrent factor associated with increased sensitivity to an allergen, escalating the dose level can increase the risk of an adverse event or more severe adverse event. Therefore, the escalation can be delayed, for example until the adverse event or concurrent factor subsides. In some embodiments, the escalation is delayed for about one week or more, about two weeks or more, or about three weeks or more.

In one example, a method of adjusting a dosage of a pharmaceutical composition comprising egg white protein during oral immunotherapy for an egg allergy in a subject, comprising orally administering to the patient a first dose of the pharmaceutical composition; and orally administering to the patient a second dose of the pharmaceutical composition, wherein the second dose is reduced, skipped, or at least a portion of the dose is delayed if the patient experiences an adverse event related to the administration of the first dose; wherein the oral immunotherapy comprises (i) an up-dosing phase comprising orally administering to the patient a series of escalating doses of the egg white protein, and (ii) a maintenance phase comprising orally administering to the patient a plurality of maintenance doses comprising the egg white protein; the method comprising. Delaying the second dose can including dividing the second dose into a first portion and a second portion, wherein the first portion is administered according to a predetermined dosing schedule, and wherein the second portion is delayed relative to the predetermined dosing schedule (for example, by about 8 hours to about 16 hours, or about 8 hours to about 12 hours), if the patient experiences the adverse event related to the administration of the first dose. In some embodiments, the adverse event related to the administration of the first dose is a mild allergenic adverse event, a moderate allergenic adverse event, or a severe allergenic adverse event. In some embodiments, the first dose and the second dose are administered to the patient during the up-dosing phase, and in some embodiments the first dose and the second dose are administered to the patient during the maintenance phase.

In another example, a method of adjusting a dosage of a pharmaceutical composition comprising egg white protein during oral immunotherapy for an egg allergy in a subject, comprises orally administering to the patient a first dose of the pharmaceutical composition; and orally administering to the patient a second dose of the pharmaceutical composition, wherein the second dose is reduced or skipped if the patient experiences a concurrent factor associated with increased sensitivity to an allergen not related to the administration of the first dose, wherein the oral immunotherapy comprises (i) an up-dosing phase comprising orally administering to the patient a series of escalating doses of the egg white protein, and (ii) a maintenance phase comprising orally administering to the patient a plurality of maintenance doses comprising the egg white protein; the method comprising. The concurrent factor associated with increased sensitivity to an allergen may be, for example, an atopic disease flare-up, inflammation, an illness, or menses. In some embodiments, the first dose and the second dose are administered to the patient during the up-dosing phase of the oral immunotherapy, and in some embodiments, the first dose and the second dose are administered to the patient during the maintenance phase of the oral immunotherapy.

### Oral Immunotherapy Treatment Endpoints

The oral immunotherapy described herein is intended to desensitize the patient to egg allergens to lower the risk of an adverse allergic event due to accidental exposure to egg white proteins. The degree of desensitization to raw egg white proteins and/or cooked egg proteins (either cooked egg white proteins or whole egg proteins, such as baked egg white protein or baked whole egg protein) can be measured using a food challenge, as described above in reference to the baseline characteristics of the patient. Successful treatment can be indicated by an increase in the highest tolerated dose or an increase in the cumulative tolerated dose for raw egg white protein or cooked (or baked) egg protein after treatment compared to at the start of treatment.

In some embodiments, the patient is able to tolerate a dose of about 300 mg of raw egg white protein, about 600 mg of raw egg white protein, about 1000 mg of raw egg white protein, or about 2000 mg raw egg white protein after the end of the up-dosing phase. In some embodiments, the patient is able to tolerate a dose of about 300 mg of raw egg white protein, about 600 mg of raw egg white protein, about 1000 mg of raw egg white protein, or about 2000 mg raw egg white protein after the end of the maintenance phase. In some embodiments, the patient is able to tolerate a cumulative dose of about 444 mg of raw egg white protein, about 1044 mg of raw egg white protein, about 2044 mg of raw egg white protein, or about 4044 mg of raw egg white protein at the end of the up-dosing phase. In some embodiments, the patient is able to tolerate a cumulative dose of about 444 mg of raw egg white protein, about 1044 mg of raw egg white protein, about 2044 mg of raw egg white protein, or about 4044 mg of raw egg white protein at the end of the maintenance phase.

In some embodiments, the patient is able to tolerate a dose of about 625 mg of cooked (or baked) egg protein at the end of the up-dosing phase, when the patient was unable to tolerate a dose of about 625 mg of cooked (or baked) egg protein, about 500 mg of cooked (or baked) egg protein, about 250 mg of cooked (or baked) egg protein, or about 125 mg of cooked (or baked) egg protein, at the start of treatment. In some embodiments, the patient is able to tolerate a dose of about 625 mg of cooked (or baked) egg protein at the end of the maintenance phase, when the patient was unable to tolerate a dose of about 625 mg of cooked (or baked) egg protein, about 500 mg of cooked (or baked) egg protein, about 250 mg of cooked (or baked) egg protein, or about 125 mg of cooked (or baked) egg protein, at the start of treatment. In some embodiments, the patient is able to tolerate a cumulative dose of about 2000 mg cooked (or baked) egg protein at the end of the up-dosing phase, wherein the patient was unable to tolerate a cumulative dose of about 2000 mg of cooked (or baked) egg protein, about 1375 mg of cooked (or baked) egg protein, about 875 mg of cooked (or baked) egg protein, about 375 mg of cooked (or baked) egg protein, or about 125 mg of cooked (or baked) egg protein, at the start of treatment. In some embodiments, the patient is able to tolerate a cumulative dose of about 2000 mg cooked (or baked) egg protein at the end of the maintenance phase, wherein the patient was unable to tolerate a cumulative dose of about 2000 mg of cooked (or baked) egg protein, about 1375 mg of cooked (or baked) egg protein, about 875 mg of cooked (or baked) egg protein, about 375 mg of cooked (or baked) egg protein, or about 125 mg of cooked (or baked) egg protein, at the start of treatment.

### EXEMPLARY EMBODIMENTS

The following embodiments are exemplary and should be not considered as limiting to the inventions described herein.

Embodiment 1. A method of making an egg white protein formulation, comprising:

(a) mixing dried egg white protein powder with a first amount of a first diluent to form a first mixture;

(b) passing the first mixture through a mesh screen;

(c) mixing the first mixture with a second amount of the first diluent to form a second mixture after steps (a) and (b);

(d) mixing the second mixture with a second diluent to form a third mixture;

(e) mixing the third mixture at a higher shear force than used for mixing in step (c); and

(f) mixing the third mixture with a lubricant to form the egg white protein formulation.

Embodiment 2. The method of embodiment 1, wherein the egg white protein formulation has about 0.05 wt % to about 2.5 wt % of egg white protein.

Embodiment 3. The method of embodiment 1 or 2, wherein the egg white protein formulation has about 0.1 wt % to about 0.7 wt % egg white protein.

Embodiment 4. The method of any one of embodiments 1-3, wherein step (b) comprises passing at least a portion of the second amount of the first diluent through the mesh screen with the first mixture.

Embodiment 5. The method of any one of embodiments 1-3, wherein step (b) comprises passing the second amount of the first diluent through the mesh screen with the first mixture.

Embodiment 6. The method of any one of embodiments 1-5, wherein step (c) comprises a plurality of sub-steps, wherein each sub-step comprises (i) adding a portion of the second amount of the first diluent to the first mixture, and (ii) mixing the portion of the second amount of the first diluent and the first mixture.

Embodiment 7. The method of embodiment 6, wherein step (c) comprises three or more sub-steps.

Embodiment 8. The method of any one of embodiments 1-5, wherein step (c) comprises continuously mixing the first mixture and the second amount of the first diluent as the second amount of the first diluent is added to the first mixture.

Embodiment 9. The method of any one of embodiments 1-8, wherein the first mixture is mixed with the second amount of the first diluent in a tumble blender.

Embodiment 10. The method of any one of embodiments 1-9, wherein the second mixture is mixed with the second diluent in a tumble blender.

Embodiment 11. The method of any one of embodiments 1-10, wherein the third mixture is mixed using a conical mill.

Embodiment 12. The method of any one of embodiments 1-11, wherein the third mixture is mixed with the lubricant in a tumble blender.

Embodiment 13. The method of any one of embodiments 1-12, wherein a third amount of the first diluent is mixed with the third mixture.

Embodiment 14. The method of embodiment 13, wherein the third amount of the first diluent and the lubricant are co-mixed with the third mixture.

Embodiment 15. The method of any one of embodiments 1-14, comprising mixing the lubricant with an additional portion of the first diluent or the second diluent before mixing the lubricant with the third mixture.

Embodiment 16. The method of embodiment 15, wherein the mixture of the lubricant and the additional portion of the first diluent or the second diluent is passed through a mesh screen before the lubricant is mixed with the third mixture.

Embodiment 17. A method of making an egg white protein formulation, comprising:

(a) mixing dried egg white protein powder with a first amount of a first diluent to form a first mixture;

(b) co-sieving the first mixture with a second portion of the first diluent through a mesh screen to form a second mixture;

(c) serially diluting the second mixture using one or more additional portions of the first diluent to form a third mixture;

(d) mixing the third mixture with a second diluent to form a fourth mixture;

(e) mixing an additional portion of the first diluent or the second diluent with a lubricant to form a fifth mixture; and

(f) mixing the fourth mixture with the fifth mixture.

Embodiment 18. The method of embodiment 17, comprising mixing the second mixture prior to step (c).

Embodiment 19. The method of embodiment 17 or 18, comprising, prior to step (e), mixing the fourth mixture using a higher shear force than used to mix the third mixture with the second diluent.

Embodiment 20. The method of any one of embodiments 17-19, comprising, prior to step (f), sieving the fifth mixture.

Embodiment 21. The method of any one of embodiments 1-20, wherein the egg white protein formulation is substantially free of colloidal silicon dioxide.

Embodiment 22. The method of any one of embodiments 1-21, wherein the egg white protein formulation is about 40 wt % to about 70 wt % of the first diluent.

Embodiment 23. The method of any one of embodiments 1-22, wherein the egg white protein formulation is about 30 wt % to about 50 wt % of the second diluent.

Embodiment 24. The method of any one of embodiments 1-23, wherein the egg white protein formulation is about 0.1 wt % to about 2 wt % of the lubricant.

Embodiment 25. A method of making an egg white protein formulation, comprising:

(a) mixing dried egg white protein powder with a first amount of a first diluent to form a first mixture;

(b) mixing a second amount of the first diluent with the first mixture to form a second mixture;

(c) mixing the second mixture with a second diluent to form a third mixture;

(d) mixing the third mixture at a higher shear force than used for mixing in step (b); and

(e) mixing the third mixture with a lubricant to form the egg white protein formulation.

Embodiment 26. The method of embodiment 25, wherein the dried egg white protein powder is mixed with the first amount of the first diluent in step (a) at a higher shear force than used for mixing in step (b).

Embodiment 27. The method of embodiment 25 or 26, wherein the egg white protein formulation has about 1 wt % to about 70 wt % egg white protein.

Embodiment 28. The method of any one of embodiments 25-27, the dried egg white protein powder is mixed with the first amount of the first diluent in step (a) using a conical mill.

Embodiment 29. The method of any one of embodiments 25-28, wherein step (b) comprises two mixing sub-steps, wherein one mixing sub-step is at a higher shear force than the other mixing sub-step.

Embodiment 30. The method of any one of embodiments 25-29, wherein step (c) further comprises mixing the second mixture with an additional amount of the first diluent prior to mixing with the second diluent to form the third mixture.

Embodiment 31. The method of any one of embodiments 25-29, wherein step (c) further comprises co-mixing the second mixture with an additional amount of the first diluent and the second diluent to form the third mixture.

Embodiment 32. The method of any one of embodiments 25-31, the third mixture is mixed in step (d) using a conical mill.

Embodiment 33. The method of any one of embodiments 25-32, wherein the second amount of the first diluent and the first mixture are mixed in a tumble blender.

Embodiment 34. The method of any one of embodiments 25-33, wherein the second mixture is mixed with the second diluent in a tumble blender.

Embodiment 35. The method of any one of embodiments 25-33, wherein a third amount of the first diluent is mixed with the third mixture.

Embodiment 36. The method of embodiment 35, wherein the third amount of the first diluent and the lubricant are co-mixed with the third mixture.

Embodiment 37. The method of any one of embodiments 25-36, wherein step (e) comprises: (i) mixing a portion of the third mixture with a lubricant; (ii) passing the mixture of (i) through a mesh screen; and (iii) mixing the mixture of (ii) with an additional portion of the third mixture to form the egg white protein formulation.

Embodiment 38. The method of any one of embodiments 25-37, wherein the third mixture is mixed with the lubricant in a tumble blender.

Embodiment 39. The method of any one of embodiments 25-38, wherein the egg white protein formulation is about 9 wt % to about 85 wt % of the first diluent.

Embodiment 40. The method of any one of embodiments 25-39, wherein the egg white protein formulation is about 10 wt % to about 50 wt % of the second diluent.

Embodiment 41. The method of any one of embodiments 25-40, wherein the egg white protein formulation is about 10 wt % to about 20 wt % of the second diluent.

Embodiment 42. The method of any one of embodiments 25-41, wherein the egg white protein formulation is about 0.1 wt % to about 2 wt % of the lubricant.

Embodiment 43. The method of any one of embodiments 25-42, wherein the egg white protein formulation comprises a glidant.

Embodiment 44. The method of embodiment 43, wherein the glidant is mixed with the egg white protein powder and the first amount of the first diluent during or prior to step (a).

Embodiment 45. The method of embodiment 43 or 44, wherein the glidant is colloidal silicon dioxide.

Embodiment 46. The method of any one of embodiments 25-45, wherein the egg white protein formulation is about 2 wt % to about 70 wt % egg white protein.

Embodiment 47. The method of any one of embodiments 25-44, wherein the egg white protein formulation is substantially free of colloidal silicon dioxide.

Embodiment 48. The method of embodiment 47, wherein the egg white protein formulation is about 1 wt % to about 5 wt % egg white protein.

Embodiment 49. A method of making an egg white protein formulation, comprising:

(a) mixing dried egg white protein powder, a first diluent, and a glidant to form a first mixture;

(b) mixing a second diluent and the first mixture at a higher shear force than used for mixing in step (a) to form a second mixture;

(c) mixing the second mixture and a lubricant to form the egg white protein formulation.

Embodiment 50. The method of embodiment 49, wherein the egg white protein formulation has about 50 wt % to about 80 wt % of egg white protein.

Embodiment 51. The method of embodiment 49 or 50, wherein the dried egg white protein powder, the first diluent, and the glidant are mixed in a tumble blender.

Embodiment 52. The method of any one of embodiments 49-51, wherein the second diluent and the first mixture are mixed in step (b) using a conical mill.

Embodiment 53. The method of any one of embodiments 49-52, wherein the second mixture and the lubricant are mixed in a tumble blender.

Embodiment 54. The method of any one of embodiments 49-53, wherein a second amount of the first diluent is mixed with the second mixture.

Embodiment 55. The method of embodiment 54, wherein the second amount of the first diluent and the lubricant are co-mixed with the second mixture.

Embodiment 56. The method of any one of embodiments 49-55, wherein the glidant comprises colloidal silicon dioxide.

Embodiment 57. The method of any one of embodiments 49-56, wherein step (c) comprises: (i) mixing a portion of the second mixture and the lubricant; (ii) passing the portion of the second mixture and the lubricant through a mesh screen; and (iii) mixing (ii) with an additional portion of the second mixture to form the egg white protein formulation.

Embodiment 58. The method of any one of embodiments 1-57, wherein the egg white protein formulation is made in a lot size of about 5 kg or more.

Embodiment 59. The method of embodiment 58, wherein the egg white protein formulation is made in a lot size of about 5 kg to about 50 kg.

Embodiment 60. The method of any one of embodiments 1-59, comprising determining an egg white protein blend uniformity for the egg white protein formulation.

Embodiment 61. The method of any one of embodiments 1-60, wherein the egg white protein formulation has an egg white protein blend uniformity relative standard deviation (RSD) of about 15% or less.

Embodiment 62. The method of any one of embodiments 1-61, comprising packaging the egg white protein formulation in a plurality of dosage containers.

Embodiment 63. The method of embodiment 62, wherein the dosage containers are capsules or sachets.

Embodiment 64. The method of embodiment 62 or 63, comprising determining an egg white protein content uniformity for the plurality of dosage containers.

Embodiment 65. The method of any one of embodiments 62-64, wherein the plurality of dosage containers has an egg white protein content uniformity relative standard deviation (RDS) of about 15% or less.

Embodiment 66. The method of any one of embodiments 1-65, wherein the first diluent is pregelatinized starch.

Embodiment 67. The method of any one of embodiments 1-66, wherein the second diluent is microcrystalline cellulose.

Embodiment 68. The method of any one of embodiments 1-67, wherein the lubricant is magnesium stearate.

Embodiment 69. The method of any one of embodiments 1-68, wherein the dried egg white protein powder comprises about 50 wt % to about 90 wt % egg white protein.

Embodiment 70. The method of any one of embodiments 1-69, wherein formation of the dried egg white protein powder comprises spray drying liquid egg whites.

Embodiment 71. The method of any one of embodiments 1-70, wherein the egg white protein powder has had glucose removed.

Embodiment 72. The method of any one of embodiments 1-71, wherein the dried egg white powder has been pasteurized.

Embodiment 73. The method of any one of embodiments 1-72, wherein the dried egg white protein powder is derived from a chicken egg.

Embodiment 74. The method of anyone of embodiments 1-73, further comprising characterizing ovomucoid, ovalbumin, ovotransferrin, or lysozyme in the dried egg white protein powder.

Embodiment 75. The method of anyone of embodiments 1-74, further comprising characterizing ovomucoid, ovalbumin, ovotransferrin, or lysozyme in the egg white protein formulation.

Embodiment 76. The method of embodiment 74 or 75, wherein characterizing ovomucoid, ovalbumin, ovotransferrin, or lysozyme comprises obtaining a high-performance liquid chromatography (HPLC) profile.

Embodiment 77. The method of embodiment 76, wherein the HPLC profile is a revere-phased HPLC (RP-HPLC) profile.

Embodiment 78. The method of embodiment 76, wherein the HPLC profile is a size-exclusion chromatography HPLC (SEC-HPLC) profile.

Embodiment 79. The method of any one of embodiments 76-78, comprising comparing the obtained HPLC profile to a reference HPLC profile.

Embodiment 80. The method of any one of embodiments 74-79, wherein characterizing ovomucoid, ovalbumin, ovotransferrin, or lysozyme comprises quantifying an amount of ovomucoid, ovalbumin, ovotransferrin, or lysozyme.

Embodiment 81. The method of embodiment 80, wherein quantifying the amount of ovomucoid, ovalbumin, ovotransferrin, or lysozyme comprises measuring an amount of ovomucoid, ovalbumin, ovotransferrin, or lysozyme compared to total protein in the egg white protein powder or the egg white protein formulation.

Embodiment 82. The method of embodiment 80, wherein quantifying the amount of ovomucoid, ovalbumin, ovotransferrin, or lysozyme comprises measuring an amount of ovomucoid, ovalbumin, ovotransferrin, or lysozyme compared to a total amount of ovomucoid, ovalbumin, ovotransferrin, and lysozyme in the egg white protein powder or the egg white protein formulation.

Embodiment 83. The method of any one of embodiments 74-82, wherein characterizing ovomucoid, ovalbumin, ovotransferrin, or lysozyme comprises measuring a potency of ovomucoid, ovalbumin, ovotransferrin, or lysozyme in the egg white protein powder or the egg white protein formulation.

Embodiment 84. The method of embodiment 83, wherein the potency of ovomucoid, ovalbumin, ovotransferrin, or lysozyme in the egg white protein powder or the egg white protein formulation is measured relative to a potency of ovomucoid, ovalbumin, ovotransferrin, or lysozyme in a reference sample.

Embodiment 85. The method of embodiment 83 or 84, wherein the potency of ovomucoid, ovalbumin, ovotransferrin, or lysozyme is measured using an immunoassay.

Embodiment 86. The method of embodiment 85, wherein the immunoassay comprises the use of one or more of an antibody that specifically binds ovomucoid, an antibody that specifically binds ovalbumin, an antibody that specifically binds ovotransferrin, or an antibody that specifically binds lysozyme.

Embodiment 87. The method of embodiment 85, wherein the immunoassay comprises the use of a pool of antibodies comprising two or more antibodies selected from the group consisting of an antibody that specifically binds ovomucoid, an antibody that specifically binds ovalbumin, an antibody that specifically binds ovotransferrin, and an antibody that specifically binds lysozyme.

Embodiment 88. The method of embodiment 86 and 87, wherein the antibody is an IgE antibody or an IgG antibody.

Embodiment 89. The method of any one of embodiments 84-88, wherein the potency of ovomucoid, ovalbumin, ovotransferrin, or lysozyme is measured using an enzyme-linked immunosorbent assay (ELISA).

Embodiment 90. An egg white protein formulation made according to the method of any one of embodiments 1-89.

Embodiment 91. An egg white protein formulation, comprising dried egg white protein powder, a first diluent, a second diluent, and a lubricant, wherein the egg white protein formulation is substantially free of colloidal silicon dioxide.

Embodiment 92. The egg white protein formulation of embodiment 91, wherein the egg white protein formulation comprises about 0.1 wt % to about 3.5 wt % egg white protein.

Embodiment 93. The egg white protein formulation of embodiment 91 or 92, wherein the first diluent is pregelatinized starch.

Embodiment 94. The egg white protein formulation of any one of embodiments 91-93, wherein the second diluent is microcrystalline cellulose.

Embodiment 95. The egg white protein formulation of any one of embodiments 91-94, wherein the lubricant is magnesium stearate.

Embodiment 96. The egg white protein formulation of any one of embodiments 91-95, wherein the egg white protein formulation consists essentially of the egg white protein powder, the first diluent, the second diluent, and the lubricant.

Embodiment 97. A method of treating an egg allergy in a patient, comprising:

orally administering to the patient a plurality of doses of a pharmaceutical composition comprising egg white protein according to an oral immunotherapy schedule comprising:

(a) an up-dosing phase comprising orally administering to the patient a series of escalating doses of about 1 mg to about 300 mg of egg white protein, wherein a given dose is administered to the patient for at least two weeks before the dose is escalated, and wherein the up-dosing phase is about 20 weeks to about 44 weeks in length; and

(b) a maintenance phase comprising orally administering to the patient a plurality of maintenance doses comprising egg white protein, wherein the maintenance phase is about 12 weeks in length or more.

Embodiment 98. The method of embodiment 97, wherein the patient has an egg-white-specific serum IgE (ew-IgE) level of about 7 kUA/L or more at the start of treatment.

Embodiment 99. The method of embodiment 97, wherein the patient has an egg-white-specific serum IgE (ew-IgE) level of about 5 kUA/L or more at the start of treatment.

Embodiment 100. The method of any one of embodiments 97-99, wherein the series of escalating doses administered to the patient during the up-dosing phase comprises at least a 1 mg dose of egg white protein and a 300 mg dose of egg white protein.

Embodiment 101. The method of any one of embodiments 97-100, wherein the series of escalating doses administered to the patient during the up-dosing phase comprises at least 10 different doses of egg white protein.

Embodiment 102. The method of any one of embodiments 97-101, wherein the series of escalating doses administered to the patient during the up-dosing phase comprises doses of about 1 mg, about 3 mg, about 6 mg, about 12 mg, about 20 mg, about 40 mg, about 80 mg, about 120 mg, about 160 mg, about 200 mg, about 240 mg, and about 300 mg of egg white protein.

Embodiment 103. The method of any one of embodiments 97-102, wherein a dose administered during the up-dosing phase is escalated only if the patient tolerates the previous dose.

Embodiment 104. The method of any one of embodiments 97-103, wherein the maximum dose administered to the patient during the up-dosing phase is about 300 mg of egg white protein.

Embodiment 105. The method of any one of embodiments 97-104, wherein the maintenance dose administered to the patient during the maintenance phase is about 300 mg of egg white protein or more.

Embodiment 106. The method of any one of embodiments 97-105, wherein the maintenance dose administered to the patient during the maintenance phase is about 300 mg of egg white protein.

Embodiment 107. The method of any one of embodiments 97-106, wherein the maintenance dose is administered to the patient only if the patient tolerates the maximum dose administered to the patient during the up-dosing phase.

Embodiment 108. The method of any one of embodiments 97-107, wherein the patient tolerates a dose of about 600 mg raw egg white protein at the end of the maintenance phase.

Embodiment 109. The method of any one of embodiments 97-108, wherein the patient tolerates a dose of about 1000 mg raw egg white protein at the end of the maintenance phase.

Embodiment 110. The method of any one of embodiments 97-109, wherein the patient tolerates a dose of about 2000 mg raw egg white protein at the end of the maintenance phase.

Embodiment 111. The method of any one of embodiments 97-110, wherein the patient tolerates a cumulative dose of about 2000 mg cooked egg white protein at the end of the maintenance phase.

Embodiment 112. The method of any one of embodiments 97-111, wherein the patient tolerates a cumulative dose of about 2000 mg baked egg white protein at the end of the maintenance phase.

Embodiment 113. The method of any one of embodiments 97-112, wherein the patient is unable to tolerate a dose of about 300 mg of raw egg white protein prior to the start of treatment.

Embodiment 114. The method of any one of embodiments 97-113 wherein the patient is unable to tolerate a cumulative dose of about 2000 mg of cooked egg white protein prior to the start of treatment.

Embodiment 115. The method of any one of embodiments 97-114, wherein the patient is unable to tolerate a cumulative dose of about 2000 mg of baked egg white protein prior to the start of treatment.

Embodiment 116. The method of any one of embodiments 97-113, wherein the patient tolerates a cumulative dose of about 2000 mg of cooked egg white protein prior to the start of treatment.

Embodiment 117. The method of any one of embodiments 97-113 and 116, wherein the patient tolerates a cumulative dose of about 2000 mg of baked egg white protein prior to the start of treatment.

Embodiment 118. The method of any one of embodiments 97-117, wherein the oral immunotherapy schedule comprises an initial escalation phase prior to the up-dosing phase, the initial escalation phase comprising orally administering to the patient a series of escalating doses of about 0.2 mg to about 2 mg of egg white protein in a single day, wherein a single administration of any given dose is administered to the patient, and wherein the doses are spaced by at least 15 minutes.

Embodiment 119. The method of embodiment 118, wherein the patient is treated according to the oral immunotherapy schedule only if the patient tolerates a dose of about 1.0 mg of raw egg white protein on the first day of treatment.

Embodiment 120. The method of any one of embodiments 97-119, wherein the patient is about 4 years of age or older prior to the start of treatment.

Embodiment 121. The method of any one of embodiments 97-120, wherein the patient is about 4 years to about 26 years of age prior to the start of treatment.

Embodiment 122. A method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising:

orally administering to the patient a first dose of the pharmaceutical composition; and

orally administering to the patient a second dose of the pharmaceutical composition, wherein the second dose is reduced, skipped, or at least a portion of the dose is delayed if the patient experiences an adverse event related to the administration of the first dose.

Embodiment 123. The method of embodiment 122, wherein the second dose is divided into a first portion and a second portion, wherein the first portion is administered according to a predetermined dosing schedule, and wherein the second portion is delayed relative to the predetermined dosing schedule, if the patient experiences the adverse event related to the administration of the first dose.

Embodiment 124. The method of embodiment 123, wherein the second portion is delayed by about 8 hours to about 12 hours after the first portion is administered.

Embodiment 125. The method of embodiment 122, wherein the second dose is skipped if the patient experiences the adverse event related to the administration of the first dose.

Embodiment 126. The method of embodiment 122, wherein the second dose is reduced relative to the first dose if the patient experiences the adverse event related to the administration of the first dose.

Embodiment 127. The method of embodiment 126, wherein subsequent doses of the pharmaceutical composition are reduced relative to the first dose for about one week or more prior to escalating the subsequent doses.

Embodiment 128. The method of embodiment 126 or 127, wherein subsequent doses of the pharmaceutical composition are reduced relative to the first dose for about one week to about two weeks prior to attempting to escalate the subsequent doses.

Embodiment 129. The method of any one of embodiments 126-128, wherein subsequent doses of the pharmaceutical composition are reduced relative to the first dose for about one week to about two weeks prior to escalating the subsequent doses.

Embodiment 130. The method of any one of embodiments 122-129, wherein the adverse event related to the administration of the first dose is a mild allergenic adverse event.

Embodiment 131. The method of any one of embodiments 122-130, wherein the adverse event related to the administration of the first dose is a moderate allergenic adverse event or a severe allergenic adverse event.

Embodiment 132. The method of any one of embodiments 122-131, wherein the first dose and the second dose are administered to the patient during the up-dosing phase of the oral immunotherapy.

Embodiment 133. The method of any one of embodiments 122-131, wherein the first dose and the second dose are administered to the patient during the maintenance phase of the oral immunotherapy.

Embodiment 134. A method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising:

orally administering to the patient a first dose of the pharmaceutical composition; and

orally administering to the patient a second dose of the pharmaceutical composition, wherein the second dose is reduced or skipped if the patient experiences a concurrent factor associated with increased sensitivity to an allergen that is not related to the administration of the first dose.

Embodiment 135. The method of embodiment 134, wherein the concurrent factor associated with increased sensitivity to an allergen is an atopic disease flare-up, inflammation, an illness, or menses.

Embodiment 136. The method of embodiment 134 or 135, wherein the second dose is skipped if the patient experiences the concurrent factor associated with increased sensitivity to an allergen not related to the administration of the first dose.

Embodiment 137. The method of embodiment 134 or 135, wherein the second dose is reduced relative to the first dose if the patient experiences the concurrent factor associated with increased sensitivity to an allergen not related to the administration of the first dose.

Embodiment 138. The method of embodiment 137, wherein subsequent doses of the pharmaceutical composition are reduced relative to the first dose for about one week or more prior to escalating the subsequent doses.

Embodiment 139. The method of embodiment 137 or 138, wherein subsequent doses of the pharmaceutical composition are reduced relative to the first dose for about one week to about two weeks prior to attempting to escalate the subsequent doses.

Embodiment 140. The method of any one of embodiments 137-139, wherein subsequent doses of the pharmaceutical composition are reduced relative to the first dose for about one week to about two weeks prior to escalating the subsequent doses.

Embodiment 141. The method of any one of embodiments 134-140, wherein the first dose and the second dose are administered to the patient during the up-dosing phase of the oral immunotherapy.

Embodiment 142. The method of any one of embodiments 134-141, wherein the first dose and the second dose are administered to the patient during the maintenance phase of the oral immunotherapy.

Embodiment 143. The method of any one of embodiments 134-142, wherein the concurrent factor associated with increased sensitivity to an allergen is an unintended exposure to a food that the patient is allergic to.

Embodiment 144. The method of any one of embodiments 97-143, wherein the egg white protein in the pharmaceutical composition is raw egg white protein.

Embodiment 145. The method of any one of embodiments 97-144, wherein the pharmaceutical composition is mixed with a food vehicle prior to administration.

Embodiment 146. A pharmaceutical composition for use in the method of any one of embodiments 97-145.

Embodiment 147. The pharmaceutical composition of embodiment 146, wherein the pharmaceutical composition comprises the egg white protein formulation prepared according to the method of any one of embodiments 1-96.

Embodiment 148. A pharmaceutical composition for use in the manufacture of a medicament for a method of treating an egg allergy in a patient according to the method of any one of embodiments 97-145.

Embodiment 149. The pharmaceutical composition of embodiment 148, wherein the pharmaceutical composition comprises the egg white protein formulation prepared according to the method of any one of embodiments 1-96.

### Examples

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1: Characterization of Dried Egg White Protein Powder

Lots of dried egg white protein powder were obtained from a commercial provider and analyzed to assess the quality of the powder for use in the manufacture of egg white protein formulations. Certain methods of characterization include comparing samples from one or more lots of the dried egg white protein powder to a Reference Standard. The Reference Standard is a sample of dried egg white protein powder monitored over time and found to have stable characteristics.

Protein content in the dried egg white protein powder was measured by a bicinchoninic acid (BCA) assay, a total protein quantitation method. Samples were prepared in water, centrifuged, and the supernatant filtered. The supernatants were then added to a 96-well microplate, and BCA reagent was added to each well. The plates, which contain aliquots of the sample and several dilutions of a reference standard was developed at 37° C. Absorbance was measured at 562 nm, and the sample absorbance was evaluated against a reference standard curve to determine the concentration of protein in the dried egg white protein powder samples. Protein content reported in a percentage of weight of protein in the dried egg white protein powder compared to total weight of dried egg white protein powder is reported in Table 3.

TABLE 3
Total Protein in Drug Substance Lots

| | Lot A | Lot B |
|---|---|---|
| Protein Content (wt %) | 75% | 75% |

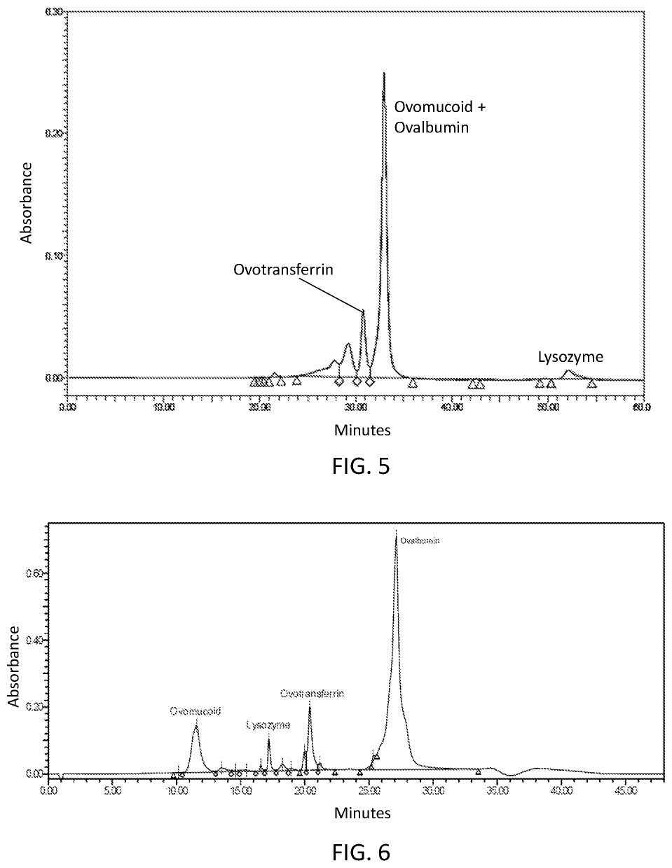

Size exclusion chromatography (SEC-HPLC) was used as one method to establish an HPLC profile for the allergenic proteins, as well as an orthogonal method to determine protein content of the dried egg white protein powder. The mobile phase was a phosphate buffered saline solution (pH 6.8) that was also used to solubilize the dried egg white powder samples. Absorbance of the column elution was measured at 220 nm. The identity of the peaks had previously been determined by injection of commercially available, purified reference products for the four egg white allergenic proteins: ovalbumin (OVA), ovomucoid (OVM), ovotransferrin (OVT), and lysozyme (LYS). The SEC-HPLC chromatogram is shown in FIG. 5, with ovotransferrin, ovomucoid, ovalbumin, and lysozyme peaks identified. The remaining peaks have not been identified. Table 4 gives the peak area percent for the four egg white allergen peaks for two lots dried egg white protein powder. While ovomucoid and ovalbumin have different molecular weights as determined from their amino acid sequence, they co-elute using this SEC-HPLC method. This may result from the protein glycosylation and protein structure giving them a similar apparent molecular weight and retention time.

TABLE 4
Peak Area Percentage in SEC-HPLC chromatogram

| Egg White Protein | Lot C | Lot D |
|---|---|---|
| Ovotransferrin | 11 | 13 |
| Ovalbumin and Ovomucoid | 66 | 75 |
| Lysozyme | 3.4 | Not integrated |

To enhance resolution and allow physical separation of the allergens in egg white, a reversed-phase HPLC (RP-HPLC) method was developed. The RP-HPLC method was based on a reversed phase separation using a wide pore 200 Å C4 column. A binary mobile phase gradient (mobile phase A: 0.05% TFA in water; mobile phase B: 0.05% TFA and 5% water in acetonitrile) was employed to accomplish baseline separation of the allergenic proteins in the dried egg white protein powder. Data were collected with a UV detector at a wavelength of 210 nm. Chromatographic peaks were identified by comparing the chromatograph peaks from the dried egg white protein powder sample against commercially purified ovotransferrin, ovalbumin, ovomucoid and lysozyme protein standards. As shown in FIG. 6, the RP-HPLC resolved the ovotransferrin, ovalbumin, ovomucoid, and lysozyme chromatographic peaks. The relative amounts of each of ovomucoid, lysozyme, ovotransferrin, and ovalbumin (compared to the amount of total protein) in a dried egg white protein powder Reference Standard, long with Lot A and Lot B of the dried egg white protein powder, as determined by chromatographic peak area are show in in Table 5.

TABLE 5
Relative Amount of
Allergenic Egg White Proteins (Percent Peak Area)

| | Reference | | |
|---|---|---|---|
| | Standard | LOT A | LOT B |
| Ovomucoid | 12% | 12% | 11% |
| Lysozyme | 2% | 2% | 2% |
| Ovotransferrin | 8% | 7% | 8% |
| Ovalbumin | 72% | 73% | 73% |

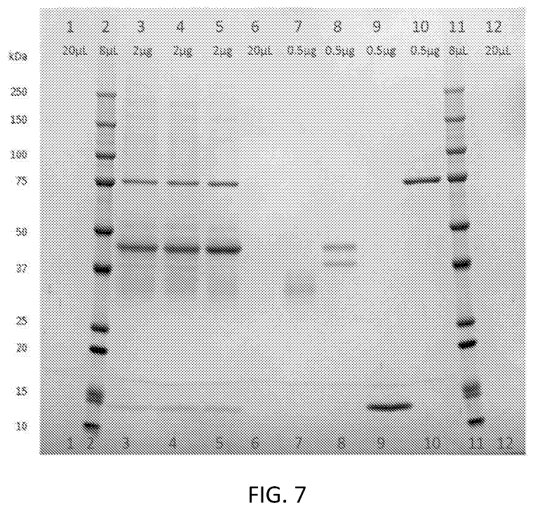

The allergenic egg white proteins were further characterized by SDS-PAGE. Proteins in the dried egg white protein powder were extracted with water, reduced using dithiothreitol, and separated using a tris-glycine gel (4-20% gradient). Protein bands were detected using Coomassie blue stain. Purified egg white protein allergens (ovomucoid, ovalbumin, lysozyme, and ovotransferrin) were obtained from commercial sources and used as comparison standards for the analysis. The SDS-PAGE data (FIG. 7) confirmed the presence of the 4 egg white protein allergen components in the analyzed samples, providing additional protein profile data to the SEC-HPLC and RP-HPLC data. Additionally, the staining pattern and intensity of all bands were consistent between the lots. The sample load for the SDS-PAGE gel shown in FIG. 7 is listed in Table 6.

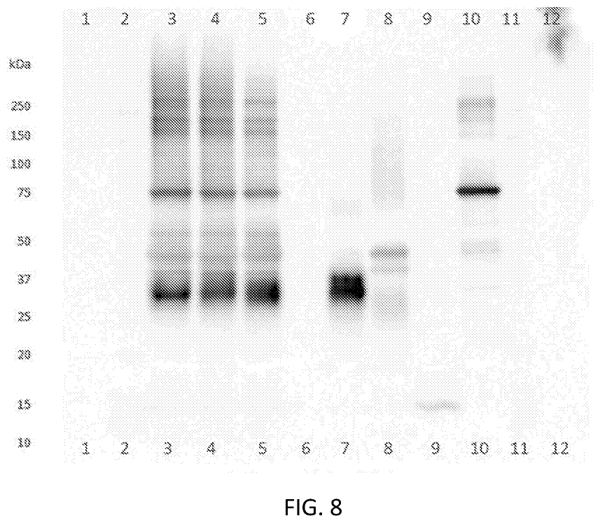

TABLE 6
Samples Loaded into SDS-PAGE gel of FIG. 7 and Immunoblot
of FIG. 8

| | | | | | | |
|---|---|---|---|---|---|---|
| Lane | 1 | 2 | 3 | 4 | 5 | 6 |
| Sample | Buffer | MWM | Lot A | Ref. Std. | Lot D | Buffer |
| Lane | 7 | 8 | 9 | 10 | 11 | 12 |
| Sample | OVM | OVA | LYS | OVT | MWM | Buffer |

MWM, molecular weight marker; Ref. Std., Reference Standard; OVM, ovomucoid; OVA, ovalbumin; LYS, lysozyme OVT, ovotransferrin.

An immunoblot analysis of the dried egg white protein powder was also performed to further characterize the allergenic egg white proteins in the dried egg white protein powder. As SDS-PAGE gel was formed as discussed above (see Table 6). The separated protein bands were transferred from the gel to a PVDF membrane, and the membrane was blocked with a 5% skim milk blocking buffer. The membrane was then treated with pooled sera from rabbits immunized with one of ovalbumin, ovomucoid, ovotransferrin, or lysozyme. As seen in FIG. 8 for the three lots of dried egg white protein powder, the protein band distribution and intensity are consistent among the lots, with bands at the expected molecular weight and with a similar pattern of immunoreactivity for the egg white protein allergens.

An ELISA was used to determine the relative potency of ovomucoid in the dried egg white protein powder from Lot A and Lot B, relative to the Reference Standard. The protein in the dried egg white protein powder samples were extracted in phosphate buffered saline (PBS) at room temperature, vortexed, centrifuged, and filtered prior to serial dilution and adsorption onto the ELISA plate. The plates were then blocked with 5% skim milk, washed, and incubated with primary antibodies (rabbit anti-ovomucoid IgG). Sequentially, the plates are again washed, a secondary antibody conjugated to horseradish peroxidase was introduced, followed by TMB substrate added. The extent of anti-ovomucoid binding to ovomucoid in the samples was determined by a colorimetric reaction. Data was fit with a logistic curve-fitting equation, e.g., 4-parameter curve fit, and EC₅₀ values were determined. The EC₅₀ value reflects the potency of the ovomucoid in the dried egg white protein powder lots, and relative potency of dried egg white protein powder was calculated based on the ratio of the EC₅₀ values (Table 7). Specifically, the 4-parameter curve fitting equation used was y=(A−D)/(1+(X/C)^{B})+D, wherein A is the minimum asymptote, B is the change in the slope of the curve, C is the inflection point of the curve (C represents the half maximal effective concentration (EC₅₀) corresponding to a response midway between the lower and upper asymptotes) and D is the maximum asymptote. The relative potency of a sample is found by dividing the EC₅₀ of a reference standard by the EC₅₀ of the sample. The reference standard may be, for example, from a previously-validated lot of dried egg white protein powder or a previously-validated lot of the pharmaceutical composition.

TABLE 7
Relative Potency of Dried Egg White Protein Powder Lots

| Lot | Relative Potency |
|---|---|
| A | 1.0 |
| B | 1.2 |

Particle size distribution of the dried egg white protein powder lots was measured using laser diffraction. The dried egg white protein powder was dispersed in methanol, pumped through a flow cell, and analyzed by laser diffraction. D₅₀ (median particle diameter, by particle volume), D₁₀ (the particle diameter at the 10th percentile of particles, by particle volume), and D₉₀ (the particle diameter at the 90th percentile of particles, by particle volume) was determined, and is presented in Table 8.

TABLE 8
Particle Size Distribution of Dried Egg White Protein
Powder

| Particle | Reference | | |
|---|---|---|---|
| Size | Standard | Lot A | Lot B |
| D₁₀ (μm) | 15 | 14 | 12 |
| D₅₀ (μm) | 77 | 74 | 66 |
| D₉₀ (μm) | 170 | 166 | 162 |

Water activity of the dried egg white protein powder was measured by placing 5 g of the dried egg white protein powder in a sample cup and placing it in a water activity instrument (AquaLab 4TEV). Within the sealed chamber of the instrument, the liquid phase and the vapor phase of water in the sample are equilibrated to give a_{w}, the relative humidity of the headspace, at 25° C. Results of this assay are shown in Table 9.

TABLE 9
Water Activity of Dried Egg White Protein Powder

| | Reference | | |
|---|---|---|---|
| | Standard | Lot A | Lot B |
| Water | 0.354 | 0.371 | 0.381 |
| Activity | | | |
| (a_{w} at 25° C.) | | | |

### Example 2: Assessment of Excipients for Egg White Protein Formulations

During formulation and process development, five lots of egg white protein formulations were manufactured and encapsulated in capsule dosage forms. The formulations were manufactured to produce 200 mg or 300 mg dosages in capsules sized 0 or 00. The contents of each formulation and measured characteristics are shown in Table 10. The percent deliverable mass of egg white protein formulation from a sampling of capsules.

TABLE 10
Summary of Egg White Protein Formulations and Capsule
Characterization

| | | Lot Number | | | | | |
|---|---|---|---|---|---|---|---|
| | | 1 | 2 | 3 | 4 | 5 | 6 |
| Nominal Dose Strength (mg) | | 200 | 300 | 300 | 300 | 300 | 300 |
| Capsule Shell Size | | #0 | #0 | #0 | #00 | #00 | #00 |
| Composition | Dried Egg White | YES | YES | YES | YES | YES | YES |
| | Microcrystalline | YES | YES | NO | YES | YES | YES |
| | Cellulose | | | | | | |
| | Pregelatinized | YES | YES | YES | YES | YES | YES |
| | Starch | | | | | | |
| | Mannitol | NO | NO | YES | NO | NO | NO |
| | Magnesium | YES | YES | YES | YES | YES | YES |
| | Stearate | | | | | | |
| | Colloidal Silicon | NO | NO | NO | YES | YES | YES |
| | Dioxide | | | | | | |

| | | | | | | |
|---|---|---|---|---|---|---|
| Average Deliverable Mass (%) | 92 | 91 | 94 | 99 | 99 | 100 |

When reopening capsules to deliver the dried egg white protein formulation contents for content uniformity testing, an average of approximately 6 wt % to 9 wt % of the formulation remain adhered to the interior of the reopened capsules when colloidal silicon dioxide was not included in the formulation (Lots 1-3). Additionally, at-line observations were recorded during encapsulation of these lots that dried egg white protein formulation remained adhered within both ends of reopened capsule shells when re-opening and shaking capsules to simulate delivery of powder contents.

Colloidal silicon dioxide was subsequently added to the blend formulation for the 300 mg dosage strengths as a glidant to improve powder flow and deliverable mass from the capsule shells during administration. The addition of colloidal silicon dioxide resulted in significant improvement in the ease with which powder can be delivered from the pull-apart capsule shells. Average deliverable mass ranged from 99 wt % to 100 wt % for subsequent formulation and process development lots of the 300 mg dosage strength (Lots 4-6). Delivered powder from reopened capsules was free-flowing with some soft agglomerates (from tamping during encapsulation) which turn to powder when gently pinched between two fingers.

### Example 3: Manufacture of Egg White Protein Formulations

Dried egg white protein formulations for dosage forms with a label claim of 0.2 mg, 1 mg, 6 mg, 12 mg, and 300 mg were manufactured, and dried egg white protein formulations for dosage forms with a label claim of 3 mg, 20 mg, 40 mg, 80 mg, 120 mg, 160 mg, 200 mg, and 240 mg will be manufactured in 7 kg lots. The nominal protein content of the dried egg white protein formulation was 80 wt %, with adjustments made to the amount of pregelatinized starch for variations in the protein content of the dried egg white protein formulation.

To assess the adequacy of the manufacturing process, blend uniformity was assessed after the final mixing step for the representative lots manufactured at the 7 kg scale. A sample thief was used to take formulation samples equivalent to 1 to 3 capsules from the manufactured formulation at 10 different locations. The protein content of each sample from the set was determined using the bicinchoninic acid (BCA) assay. Results are summarized in Table 11.

TABLE 11
Blend Uniformity Results for Representative Lots

| Dosage Strength | 0.2 mg | 1 mg | 6 mg | 12 mg | 300 mg |
|---|---|---|---|---|---|
| Average (% LC) | 83 | 90 | 101 | 104 | 102 |
| RSD (%) | 14 | 2 | 6 | 2 | 1 |
| Minimum (% LC) | 68 | 86 | 84 | 102 | 101 |
| Maximum (% LC) | 113 | 94 | 105 | 108 | 103 |

% LC: Percent of Label Claim

The egg white protein formulations for 0.2 mg, 1 mg, 6 mg, 12 mg, and 300 mg label claims were filled into pull-apart hypromellose-based capsule shells to supply individual measured doses. Size #2 pull-apart capsule shells with a target powder fill weight of 180 mg were selected for the 0.2 to 6 mg dosage strengths. Size #00 capsule shells with a target fill weight of 500 mg were selected for the higher dosage strengths to accommodate up to 300 mg of egg white protein with sufficient diluent, lubricant, and glidant to allow for robust processing and deliverable mass. A Bosch GKF701 encapsulator was used to encapsulate the egg white protein formulation in capsules at production rates up to 700 capsules per minute. The formulation was fed to the encapsulator and tamped into a dosing disk using a series of tamping pins. The dosing disk thickness and tamping pin positions can be adjusted to modulate the capsule fill weight. Empty capsule shells were opened by the encapsulator, a tamped slug of powder is inserted into the body of each opened capsule shell, and then the capsule shell was closed.

The percent deliverable mass of the capsules was measured by sampling a set of ten manufactured capsules at 0.2 mg, 1 mg, 6 mg, 12 mg, and 300 mg dose claims. The capsules were weighed, opened, and the formulation contained within the capsule was poured out. The mass of the formulation deliverable from the capsule was measured. Compressed air was then blown into the capsule to remove residual formulation, and the empty capsule was weighed. The percent delivered mass and the RSD for each dose claim are summarized in Table 12.

TABLE 12
Deliverable Mass Summary for Representative Lots

| Nominal Dose (mg) | | |
|---|---|---|
| (Label Claim) | % Delivered (n = 10) | % RSD (n = 10) |
| 0.2 | 100.0 | 0.4 |
| 1 | 99.0 | 0.5 |
| 6 | 98.6 | 0.7 |
| 12 | 95.5 | 0.2 |
| 300 | 100.0 | 0.2 |

Content uniformity of the capsules was also determined by comparing the protein content in the deliverable mass to the nominal dose (i.e., label claim). The protein content was determined using the BCA assay. The content uniformity of the lots of dosage containers at the various doses is summarized in Table 13.

TABLE 13
Content Uniformity Summary for Representative Lots

| Nominal Dose (mg) | | |
|---|---|---|
| (Label Claim) | % Delivered (n = 10) | % RSD (n = 10) |
| 0.2 | 92 | 9 |
| 1 | 93 | 5 |
| 6 | 100 | 4 |
| 12 | 97 | 3 |
| 300 | 106 | 3 |

### Example 4: Oral Immunotherapy Study for the Treatment of an Egg Allergy

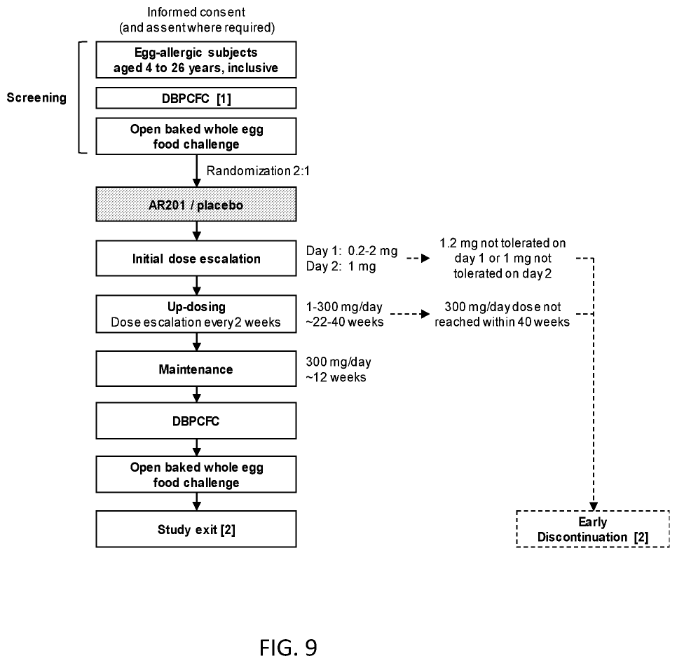

A randomized, double-blind, placebo-controlled study of the efficacy and safety of a pharmaceutical composition containing raw, dried egg white protein powder derived from hen eggs and excipients in an oral immunotherapy regimen will be undertaken. A placebo formulated with an inactive compound and the corresponding excipients will be used for a placebo arm of the study. A schematic of the study is provided in FIG. 9.

Eligible subjects are human individuals that (1) are aged 4 to 26 years of age; (2) have a physician-diagnosed IgE-mediated hen egg allergy that develop an allergenic reaction within 2 hours of known oral exposure to egg or egg-containing food; (3) have a serum egg white specific IgE level of >7 kU_{A}/L; (4) develop dose-limiting allergy symptoms after consuming single doses of <300 mg dried egg white protein in a screening double-blind placebo-controlled food challenge (DBPCFC); (5) for female individuals of childbearing potential, are using a reliable birth control method; and (6) are not otherwise excluded according to exclusion criteria.

Exclusion criteria include (1) a history of severe or life-threatening anaphylaxis or anaphylactic shock within 60 days before screening; (2) a history of eosinophilic esophagitis (EoE) or other eosinophilic GI disease; chronic, recurrent, or severe gastroesophageal reflux disease (GERD); symptoms of dysphagia; recurrent GI symptoms of any etiology; (3) a history of a mast cell disorder (e.g., systemic mastocytosis, urticaria pigmentosa, chronic idiopathic or chronic physical urticaria beyond simple dermatographism [e.g., cold urticaria, cholinergic urticaria], or hereditary or idiopathic angioedema); (4) have mild or moderate asthma that is uncontrolled or difficult to control, or severe persistent asthma; (5) a history of high-dose corticosteroid medication use (e.g., >3 days at 1-2 mg/kg of prednisone or equivalent); (6) a history of cardiovascular disease (including uncontrolled or inadequately controlled hypertension); (7) a history of chronic disease (except asthma, atopic dermatitis, or allergic rhinitis) that is or is at significant risk of becoming unstable or requiring a change in a chronic therapeutic regimen, including malignancies within 5 years before screening and autoimmune diseases; (8) a history of cardiovascular disease including uncontrolled or inadequately controlled hypertension; (9) use of beta-blockers (oral), angiotensin-converting enzyme inhibitors, angiotensin receptor blockers, calcium channel blockers, or tricyclic antidepressants; (10) unable to discontinue antihistamines and other medications that could interfere with the assessment of an allergic reaction for 5 half-lives of the medication before the screening and exit skin prick tests (SPTs) and food challenges, and the first day of dose escalation; (11) lack of an available palatable vehicle food to which the subject is not allergic; (12) a hypersensitivity to wheat or oat; (13) a hypersensitivity to epinephrine or any of the excipients in the epinephrine auto-injector; (14) use of any therapeutic antibody or any immunomodulatory therapy (including immunosuppressive medications (except aeroallergen or venom immunotherapy used in the maintenance phase within 6 months before screening); (15) currently receiving or received within 5 years before screening any type of egg or other food allergen immunotherapy; (16) participation in another clinical study within 30 days or 5 half-lives of the investigational product, whichever is longer, before screening; (17) in the build-up/up-dosing phase of immunotherapy for any non-egg allergen; and (18) pregnancy or currently breastfeeding.

Initial screening will include a dried egg white double-blind placebo controlled food challenge (DBPCFC) and an open baked whole egg food challenge. The DBPCFC is a procedure performed under medical supervision by feeding dried egg white and placebo in measured, increasing doses on 2 separate days with the subject, parent/caregiver, and study site staff blinded to the randomized order of the challenge days. Study site personnel will not be unblinded to the order of the challenge until after completion of both days of the DBPCFC. The food challenge material will be prepared by a designated unblinded person who is not involved in dosing, monitoring, or assessing the outcome of the DBPCFC. The DBPCFC conducted in this study will follow procedures consistent with the Practical Allergy (PRACTALL) guidelines for safety, assessment, and scoring (Sampson et al., J. Allergy Clin. Immunol., vol. 130, pp. 1260-1274 (2012)). The DBPCFC procedure is performed during screening and also at study exit. A summary of the screening and exit DBPCFC dosing schedule is provided in Table 14 below.

TABLE 14
Screening and Exit DBPCFC Challenge Doses

| | Dried Egg White | Cumulative Dose (mg) | |
|---|---|---|---|
| Timing | Protein Dose (mg) | Screening | Exit¹ |
| Screening | 1 | 1 | 0 (or 1) |
| Screening/Exit | 3 | 4 | 3 (or 4) |
| Screening/Exit | 10 | 14 | 13 (or 14) |
| Screening/Exit | 30 | 44 | 43 (or 44) |
| Screening/Exit | 100 | 144 | 143(or 144) |
| Screening/Exit | 300 | 444 | 443 (or 444) |
| Exit | 600 | N/A | 1043 (or 1044) |
| Exit | 1,000 | N/A | 2043 (or 2044) |
| Exit | 2,000 | N/A | 4043 (or 4044) |

¹The 1 mg challenge dose may be administered at the exit DBPCFC per investigator decision.

The open baked whole egg food challenge is a procedure performed under medical supervision by feeding a test food product (baked food product with egg) in measured, increasing doses. The challenge conducted under this study will follow procedures consistent with the PRACTALL guidelines for safety, assessment, and scores. The open baked whole egg food challenge is performed during screening and also at study exit. During the challenge, subjects will attempt to consume one entire muffin according to a dosing schedule, wherein one muffin contains approximately one-third of one whole egg, which is equivalent to approximately 2000 mg egg protein. A summary of the screening and exit open baked whole egg food challenge dosing schedule is provided in Table 15 below.

TABLE 15
Screening and Exit Open Baked Whole Egg Food Challenge Doses

| | | Approximate | Approximate |
|---|---|---|---|
| | | Baked | Cumulative Baked |
| | Approximate | Egg Protein | Egg Protein Dose E |
| Timing | Amount of Muffin | Dose (mg) | (mg) |
| Screening/Exit | 1/16 | 125 | 125 |
| Screening/Exit | ⅛ | 250 | 375 |
| Screening/Exit | ¼ | 500 | 875 |
| Screening/Exit | ¼ | 500 | 1375 |
| Screening/Exit | ⅓ (remainder of | 625 | 2000 |
| | muffin) | | |

Eligible individuals will be randomly assigned 2:1 to blinded treatment with the pharmaceutical composition or placebo. Randomization will be stratified by baseline reactivity to baked egg in an open baked whole egg food challenge at screening. Subjects who tolerate approximately 2000 mg cumulative baked egg protein (one muffin which contains approximately one-third of one whole egg) will be allowed to consume baked egg products during the course of the study. Subjects who have dose-limiting allergy symptoms during the open baked whole egg food challenge will be considered baked egg intolerant and will be instructed to avoid all forms of hen egg during the study.

Subjects will begin initial dose escalations under medical supervision at the study site on day 1 with a stepwise dose escalation of study product (up to 5 single doses of 0.2 mg, 0.4 mg, 0.8 mg, 1.2 mg, and 2 mg) administered at 20- to 30-minute intervals as tolerated. Subjects who tolerate at least the 1.2 mg single dose on day 1 will return on day 2 for a single confirmatory 1 mg dose. Subjects who tolerate the confirmatory 1 mg dose with no more than mild allergy symptoms that are not dose-limiting will begin the up-dosing period. Subjects who do not tolerate the 1.2 mg dose on day 1 or 1 mg dose on day 2 will discontinue early from the study.

Up-dosing will be approximately 6 months (22-40 weeks), with dose escalation occurring approximately every 2 weeks. Daily doses of study product during up-dosing will be 1 mg, 3 mg, 6 mg, 12 mg, 20 mg, 40 mg, 80 mg, 120 mg, 160 mg, 200 mg, 240 mg, and 300 mg. The first dose of study product at each new dose level will be administered under medical supervision at the study site; the remaining doses at each dose level will be administered daily at home as tolerated.

Subjects who reach the 300 mg/day dose within 40 weeks and tolerate the first 300 mg dose with no more than mild allergy symptoms that are not dose-limiting will begin the maintenance period. Subjects who do not reach the 300 mg/day dose within 40 weeks of day 1 will discontinue early from the study.

Subjects who begin maintenance treatment will continue daily dosing with study product at 300 mg/day for approximately 12 weeks, which may be extended by up to an additional 4 weeks to accommodate dose adjustments during the last 2 weeks of maintenance. Study site visits will occur approximately every 4 weeks. Study product will be administered under medical supervision at the study site during maintenance visits; subsequent maintenance doses will be administered daily at home as tolerated.

Dose adjustments during the up-dosing phase or maintenance phase will be allowed, as approved by the investigator, depending on the subject experiencing an adverse event or other concurrent factor. During either the up-dosing or maintenance phase, the dose may be adjusted in response to a dose-related allergic reaction in accordance with one of the following: (1) administer the next dose of study product at the study site under medical supervision; (2) delay the study product dose escalation an additional 1 to 2 weeks; (3) reduce the study product dose level by 1 or 2 dose levels; (4) temporarily withhold study product; or (5) stop study product dosing and discontinue the subject early from the study. The severity of symptoms will guide study product dose reductions for both acute and chronic or recurrent symptoms.

The dose may also be adjusted for reasons other than an allergic reaction caused by the pharmaceutical composition, such as a flare up of asthma or other atopic disease, an illness, or menses. The amount of dose reduction may range from 1 dose level (i.e., the previous dose level) to approximately 50% (rounded down to the nearest feasible whole dose) at the discretion of the investigator. The lowest dose level is 1 mg. If the dose is reduced for reasons other than allergic reactions to the pharmaceutical composition, the reduced dose will be given for 2 weeks and the subject is to be fully recovered (i.e., baseline status) for at least 3 days, depending on the severity of the illness per investigator assessment, before attempting dose re-escalation at the study site. Dosing will stop and the subject will discontinue early if any of the following conditions are met for dose adjustment: the dose level cannot be escalated after 3 consecutive failed attempts with at least 2 weeks between each escalation attempt; or the dose reduction cannot be tolerated after 3 attempts to reduce the dose level.

At the end of maintenance, subjects will have an exit DBPCFC up to a single highest challenge dose of 2000 mg dried egg white protein (4043 or 4044 mg cumulative), followed by an open baked whole egg food challenge within 7 days after the second day of the exit DBPCFC. All subjects must tolerate the 300 mg daily dose of study product for at least 2 consecutive weeks before having the exit DBPCFC.

Subjects who complete the exit DBPCFC and open baked whole egg food challenge will exit (complete) the study. Study treatment assignment will be unblinded for a subject after study exit and after all major data queries for the subject are resolved. For a subject who discontinues early from the study, study treatment assignment will be unblinded after the study is completed.

The studied endpoints of the study include (1) the proportion of subjects treated with the pharmaceutical composition compared with placebo who tolerate a single highest dose of at least 300 mg raw egg white protein, at least 600 mg raw egg white protein, at least 1000 mg raw egg white protein, or at least 2000 mg raw egg white protein, with no more than mild allergy symptoms at the exit DBPCFC; (2) the proportion of subjects that could not tolerate a cumulative dose of about 2000 mg baked egg protein at screening and subsequently tolerate a cumulative dose of about 2000 mg baked egg protein at study exit; and (3) safety summaries of the treatment.

### Example 5: Oral Immunotherapy Study for the Treatment of Egg Allergy

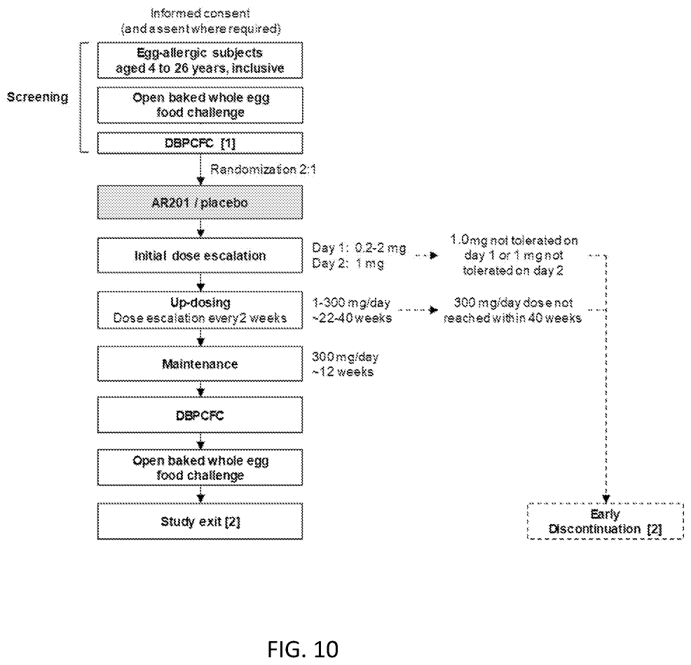

A randomized, double-blind, placebo-controlled study of the efficacy and safety of a pharmaceutical composition containing raw, dried egg white protein powder derived from hen eggs and excipients in an oral immunotherapy regimen will be undertaken. A placebo formulated with an inactive compound and the corresponding excipients will be used for a placebo arm of the study. A schematic of the study is provided in FIG. 10.

Eligible subjects are human individuals that (1) are aged 4 to 26 years of age; (2) have a physician-diagnosed IgE-mediated hen egg allergy that develop an allergenic reaction within 2 hours of known oral exposure to egg or egg-containing food; (3) have a serum egg white specific IgE level of >5 kU_{A}/L; (4) develop dose-limiting allergy symptoms after consuming single doses of <300 mg dried egg white protein in a screening double-blind placebo-controlled food challenge (DBPCFC); (5) for female individuals of childbearing potential, are using a reliable birth control method; and (6) are not otherwise excluded according to exclusion criteria.

Exclusion criteria include (1) a history of severe or life-threatening anaphylaxis or anaphylactic shock within 60 days before screening; (2) a history of eosinophilic esophagitis (EoE) or other eosinophilic GI disease; chronic, recurrent, or severe gastroesophageal reflux disease (GERD); symptoms of dysphagia; recurrent GI symptoms of any etiology; (3) a history of a mast cell disorder (e.g., systemic mastocytosis, urticaria pigmentosa, chronic idiopathic or chronic physical urticaria beyond simple dermatographism [e.g., cold urticaria, cholinergic urticaria], or hereditary or idiopathic angioedema); (4) have mild or moderate asthma that is uncontrolled or difficult to control, or severe persistent asthma; (5) a history of high-dose corticosteroid medication use (e.g., >3 days at 1-2 mg/kg of prednisone or equivalent); (6) a history of cardiovascular disease (including uncontrolled or inadequately controlled hypertension); (7) a history of chronic disease (except asthma, atopic dermatitis, or allergic rhinitis) that is or is at significant risk of becoming unstable or requiring a change in a chronic therapeutic regimen, including malignancies within 5 years before screening and autoimmune diseases; (8) a history of cardiovascular disease including uncontrolled or inadequately controlled hypertension; (9) use of beta-blockers (oral), angiotensin-converting enzyme inhibitors, angiotensin receptor blockers, calcium channel blockers, or tricyclic antidepressants; (10) unable to discontinue antihistamines and other medications that could interfere with the assessment of an allergic reaction for 5 half-lives of the medication before the screening and exit skin prick tests (SPTs) and food challenges, and the first day of dose escalation; (11) lack of an available palatable vehicle food to which the subject is not allergic; (12) a hypersensitivity to wheat or oat; (13) a hypersensitivity to epinephrine or any of the excipients in the epinephrine auto-injector; (14) use of any therapeutic antibody or any immunomodulatory therapy (including immunosuppressive medications (except aeroallergen or venom immunotherapy used in the maintenance phase within 6 months before screening); (15) currently receiving or received within 5 years before screening any type of egg or other food allergen immunotherapy; (16) participation in another clinical study within 30 days or 5 half-lives of the investigational product, whichever is longer, before screening; (17) in the build-up/up-dosing phase of immunotherapy for any non-egg allergen; and (18) pregnancy or currently breastfeeding.

Initial screening will include a dried egg white double-blind placebo controlled food challenge (DBPCFC) and an open baked whole egg food challenge. The DBPCFC is a procedure performed under medical supervision by feeding dried egg white and placebo in measured, increasing doses on 2 separate days with the subject, parent/caregiver, and study site staff blinded to the randomized order of the challenge days. Study site personnel will not be unblinded to the order of the challenge until after completion of both days of the DBPCFC. The food challenge material will be prepared by a designated unblinded person who is not involved in dosing, monitoring, or assessing the outcome of the DBPCFC. The DBPCFC conducted in this study will follow procedures consistent with the Practical Allergy (PRACTALL) guidelines for safety, assessment, and scoring (Sampson et al., J. Allergy Clin. Immunol., vol. 130, pp. 1260-1274 (2012)). The DBPCFC procedure is performed during screening and also at study exit. A summary of the screening and exit DBPCFC dosing schedule is provided in Table 3 below.

TABLE 16
Screening and Exit DBPCFC Challenge Doses

| | Dried Egg White | Cumulative Dose (mg) | |
|---|---|---|---|
| Timing | Protein Dose (mg) | Screening | Exit¹ |
| Screening | 1 | 1 | 0 (or 1) |
| Screening/Exit | 3 | 4 | 3 (or 4) |
| Screening/Exit | 10 | 14 | 13 (or 14) |
| Screening/Exit | 30 | 44 | 43 (or 44) |
| Screening/Exit | 100 | 144 | 143(or 144) |
| Screening/Exit | 300 | 444 | 443 (or 444) |
| Exit | 600 | N/A | 1043 (or 1044) |
| Exit | 1,000 | N/A | 2043 (or 2044) |
| Exit | 2,000 | N/A | 4043 (or 4044) |

¹The 1 mg challenge dose may be administered at the exit DBPCFC per investigator decision.

The open baked whole egg food challenge is a procedure performed under medical supervision by feeding a test food product (baked food product with egg) in measured, increasing doses. The challenge conducted under this study will follow procedures consistent with the PRACTALL guidelines for safety, assessment, and scores. The open baked whole egg food challenge is performed during screening and also at study exit. During the challenge, subjects will attempt to consume one entire muffin according to a dosing schedule, wherein one muffin contains approximately one-third of one whole egg, which is equivalent to approximately 2000 mg egg protein. A summary of the screening and exit open baked whole egg food challenge dosing schedule is provided in Table 4 below.

TABLE 17
Screening and Exit Open Baked Whole Egg Food Challenge Doses

| | | Approximate | Approximate |
|---|---|---|---|
| | Approximate | Baked Egg | Cumulative Baked |
| | Amount of | Protein Dose | Egg Protein Dose |
| Timing | Muffin | (mg) | (mg) |
| Screening/Exit | 1/16 | 125 | 125 |
| Screening/Exit | ⅛ | 250 | 375 |
| Screening/Exit | ¼ | 500 | 875 |
| Screening/Exit | ¼ | 500 | 1375 |
| Screening/Exit | ⅓ (remainder | 625 | 2000 |
| | of muffin) | | |

Eligible individuals will be randomly assigned 2:1 to blinded treatment with the pharmaceutical composition or placebo. Randomization will be stratified by baseline reactivity to baked egg in an open baked whole egg food challenge at screening. Subjects who tolerate approximately 2000 mg cumulative baked egg protein (one muffin which contains approximately one-third of one whole egg) will be allowed to consume baked egg products during the course of the study. Subjects who have dose-limiting allergy symptoms during the open baked whole egg food challenge will be considered baked egg intolerant and will be instructed to avoid all forms of hen egg during the study.

Subjects will begin initial dose escalations under medical supervision at the study site on day 1 with a stepwise dose escalation of study product (up to 5 single doses of 0.2 mg, 0.4 mg, 0.8 mg, 1.0 mg, and 2 mg) administered at 20- to 30-minute intervals as tolerated. Subjects who tolerate at least the 1.0 mg single dose on day 1 will return on day 2 for a single confirmatory 1.0 mg dose. Subjects who tolerate the confirmatory 1.0 mg dose with no more than mild allergy symptoms that are not dose-limiting will begin the up-dosing period. Subjects who do not tolerate the 1.0 mg dose on day 1 or 1 mg dose on day 2 will discontinue early from the study.

Up-dosing will be approximately 6 months (22-40 weeks), with dose escalation occurring approximately every 2 weeks. Daily doses of study product during up-dosing will be 1 mg, 3 mg, 6 mg, 12 mg, 20 mg, 40 mg, 80 mg, 120 mg, 160 mg, 200 mg, 240 mg, and 300 mg. The first dose of study product at each new dose level will be administered under medical supervision at the study site; the remaining doses at each dose level will be administered daily at home as tolerated.

Subjects who reach the 300 mg/day dose within 40 weeks and tolerate the first 300 mg dose with no more than mild allergy symptoms that are not dose-limiting will begin the maintenance period. Subjects who do not reach the 300 mg/day dose within 40 weeks of day 1 will discontinue early from the study.

Subjects who begin maintenance treatment will continue daily dosing with study product at 300 mg/day for approximately 12 weeks, which may be extended by up to an additional 4 weeks to accommodate dose adjustments during the last 2 weeks of maintenance. Study site visits will occur approximately every 4 weeks. Study product will be administered under medical supervision at the study site during maintenance visits; subsequent maintenance doses will be administered daily at home as tolerated.

Dose adjustments during the up-dosing phase or maintenance phase will be allowed, as approved by the investigator, depending on the subject experiencing an adverse event or other concurrent factor. During either the up-dosing or maintenance phase, the dose may be adjusted in response to a dose-related allergic reaction in accordance with one of the following: (1) administer the next dose of study product at the study site under medical supervision; (2) delay the study product dose escalation an additional 1 to 2 weeks; (3) reduce the study product dose level by 1 or 2 dose levels; (4) temporarily withhold study product; or (5) stop study product dosing and discontinue the subject early from the study. The severity of symptoms will guide study product dose reductions for both acute and chronic or recurrent symptoms.

The dose may also be adjusted for reasons other than an allergic reaction caused by the pharmaceutical composition, such as a flare up of asthma or other atopic disease, an illness, or menses. The amount of dose reduction may range from 1 dose level (i.e., the previous dose level) to approximately 50% (rounded down to the nearest feasible whole dose) at the discretion of the investigator. The lowest dose level is 1 mg. If the dose is reduced for reasons other than allergic reactions to the pharmaceutical composition, the reduced dose will be given for 2 weeks and the subject is to be fully recovered (i.e., baseline status) for at least 3 days, depending on the severity of the illness per investigator assessment, before attempting dose re-escalation at the study site. Dosing will stop and the subject will discontinue early if any of the following conditions are met for dose adjustment: the dose level cannot be escalated after 3 consecutive failed attempts with at least 2 weeks between each escalation attempt; or the dose reduction cannot be tolerated after 3 attempts to reduce the dose level.

At the end of maintenance, subjects will have an exit DBPCFC up to a single highest challenge dose of 2000 mg dried egg white protein (4043 or 4044 mg cumulative), followed by an open baked whole egg food challenge within 7 days after the second day of the exit DBPCFC. All subjects must tolerate the 300 mg daily dose of study product for at least 2 consecutive weeks before having the exit DBPCFC.

Subjects who complete the exit DBPCFC and open baked whole egg food challenge will exit (complete) the study. Study treatment assignment will be unblinded for a subject after study exit and after all major data queries for the subject are resolved. For a subject who discontinues early from the study, study treatment assignment will be unblinded after the study is completed.

The studied endpoints of the study include (1) the proportion of subjects treated with the pharmaceutical composition compared with placebo who tolerate a single highest dose of at least 300 mg raw egg white protein, at least 600 mg raw egg white protein, at least 1000 mg raw egg white protein, or at least 2000 mg raw egg white protein, with no more than mild allergy symptoms at the exit DBPCFC; (2) the proportion of subjects that could not tolerate a cumulative dose of about 2000 mg baked egg protein at screening and subsequently tolerate a cumulative dose of about 2000 mg baked egg protein at study exit; and (3) safety summaries of the treatment.

### Example 6: RP-HPLC Assay

A RP-HPLC method was developed for the higher resolution identification and protein profile of dried egg white powder (the drug substance) and pharmaceutical compositions of egg white protein (the drug product).

To prepare the drug substance for assay, 50 mg of the drug substance (corresponding to about 40 mg egg white protein) is weighed into a 1000 ml flask and diluted with 800 ml of water. The sample is then shaken on an orbital shaker for 30 minutes and then diluted with water to 0.04 mg/ml egg white protein concentration. The diluted sample is then filtered through a 0.45 micron syringe filter, discarding the first 2 ml.

To prepare the pharmaceutical composition (i.e., the drug product) for assay, the volume of diluent depends on the dosage strength of the sample being tested. 10 capsules from a particular dose level may be blended together and diluted with water to about 0.05 mg/ml egg white protein concentration. The diluted drug product sample may then be shaken on an orbital shaker for 30 minutes and diluted to the working concentration of about 0.038 mg/ml to about 0.042 mg/ml egg white protein concentration. The samples may then be transferred to 15 ml conical centrifuge tubes and centrifuged at 3,200 rpm for 30 minutes. The clarified supernatant can then be filtered through a 0.45 micron syringe filter, discarding the first 2 ml.

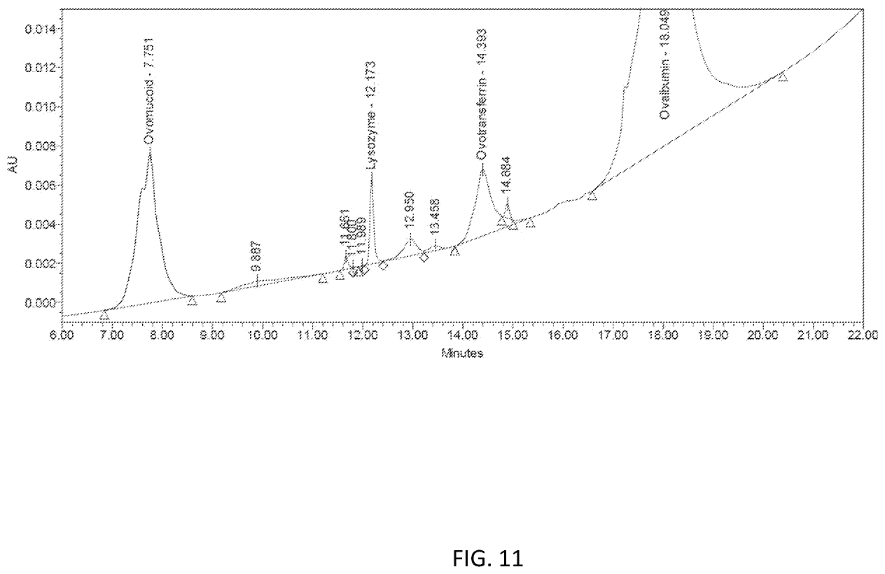

Using the improved RP-HPLC method, major egg white protein components were well-resolved with typical retentions times of about 15.5 minutes (myoglobin), 16.1 minutes (carbonic anhydrase), 7.8 minutes (ovomucoid), 12.2 minutes (lysozyme), 14.4 minutes (ovotransferrin), and 18.1 minutes (ovalbumin). A sample RP-HPLC chromatogram of the pharmaceutical formulation using the improved RP-HPLC method is depicted in FIG. 11. Analytical samples of the drug substance or drug product may be compared to reference standards of the individual proteins (i.e., purified allergens or other egg white proteins) and/or previously-validated samples of the drug substance or drug product. The diluent of the pharmaceutical formulated exhibited a broad retention profile with a maximum amplitude after 22 minutes. The peak areas for each individual protein can calculated by subtracting the area attributable to the diluent by comparison to a reference standard of the diluent and, optionally, any other excipient alone.

### Example 7: Stability Study

A stability study was undertaken with samples of the pharmaceutical formulation of egg white protein. Samples were tested under normal, accelerated, and stressed stability conditions, such as storage between 25-40° C.

In one experiment, 0.2 mg capsules were stored in high-density polyethylene bottles with a 1-g desiccant pouch. The bottles were stored at 5±3° C., 25° C./60% RH, 30° C./65% RH, or 40° C./75% RH.

At the various time points, samples were assayed for appearance, deliverable mass, protein profile (by RP-HPLC), total protein by BCA, and relative potency by ELISA. Appearance was assessed by opening individual capsules and placing a small amount of the product on a clean watch glass. The material was observed against a white background for the presence of foreign matter. Deliverable mass was assessed by weighing the weight of the intact capsule, emptying the capsule into a tared container, using compressed air to blow out remaining contents from the capsule shell, weighing the empty capsule, and then determining what percentage of the mass removed from the capsule was delivered to the tared container. Protein profile was assessed by measuring the proportion of RP-HPLC peak area was attributable to Gal d 1, Gal d 2, Gal d 3, and Gal d 4. For the earlier time points (between 1 and 6 months), the first RP-HPLC method described in Example 1 was used. For the later time points, the improved RP-HPLC method described in Example 6 was used. Both RP-HPLC methods were used for the 6-month time points. Relative potency by ELISA and total protein by BCA were assessed as described in Example 1. BCA is reported as percent of label claim of total egg white protein.

The results of the 0.2 mg capsule after 2-8° C. storage are summarized in the table, below. For the 6-month time point, both the older RP-HPLC method of Example 1 and the improved RP-HPLC of Example 6 were employed. The first value in the 6-month time point corresponds to the first RP-HPLC method, and the second value in the 6-month time point corresponds to the improved RP-HPLC method.

TABLE 18
Stability Data for Pharmaceutical Formulation (0.2 mg capsules; 2-8° C.
storage)

| | Test | | | | |
|---|---|---|---|---|---|
| | T = 0 | T = 3 | T = 6 | T = 9 | T = 12 |
| Appearance | Conforms | Conforms | Conforms | Conforms | Conforms |
| Deliverable | 100% | 98% | 99% | 99% | 100% |
| mass | | | | | |
| % Gal d 1 | 13% | 14% | 14% / 11% | 12% | 11% |
| % Gal d 2 | 78% | 77% | 77% / 80% | 79% | 79% |
| % Gal d 3 | 4% | 5% | 5% / 3% | 4% | 5% |
| % Gal d 4 | 0.5% | 0.2% | 0.3% / 2% | 2% | 1% |
| Relative | 0.8 | 1.1 | 1.2 | 1.5 | 1.0 |
| potency | | | | | |
| Total protein | 91% | 83% | 99% | 85% | 87% |
| (BCA) | | | | | |

The major allergen levels and the relative potency of the pharmaceutical formulation were stable between 0 and 12 months after storage at between 2-8° C.

The results of the 0.2 mg capsule after 25° C./60% RH accelerated stability storage condition are summarized in the table, below. For the 6-month time point, both the older RP-HPLC method and the improved RP-HPLC method described were employed. The first value in the 6-month time point corresponds to the first RP-HPLC method, and the second value in the 6-month time point corresponds to the improved RP-HPLC method.

TABLE 19
Stability Data for Pharmaceutical Formulation (0.2 mg capsules;
25° C./60% storage)

| | Test | | | | |
|---|---|---|---|---|---|
| | T = 0 | T = 3 | T = 6 | T = 9 | T = 12 |
| Appearance | Conforms | Conforms | Conforms | Conforms | Conforms |
| Deliverable | 100% | 99% | 99% | 99% | 100% |
| mass | | | | | |
| % Gal d 1 | 13% | 14% | 14% / 11% | 12% | 11% |
| % Gal d 2 | 78% | 77% | 77% / 80% | 81% | 80% |
| % Gal d 3 | 4% | 5% | 5% / 3% | 3% | 4% |
| % Gal d 4 | 0.5% | 0.3% | 0.3% / 1% | 1% | 1% |
| Relative | 0.8 | 1.4 | 1.2 | 1.2 | 1.7 |
| potency | | | | | |
| Total protein | 91% | 77% | 83% | 86% | 86% |
| (BCA) | | | | | |

The major allergen levels and the relative potency of the pharmaceutical formulation were stable between 0 and 12 months after storage at 25° C./60% RH.

The results of the 0.2 mg capsule after 30° C./65% RH accelerated stability storage condition are summarized in the table, below. For the 6-month time point, both the older RP-HPLC method and the improved RP-HPLC method described in the preceding example were employed, but only the older RP-HPLC values are reported for comparison the T=0 time point.

TABLE 20
Stability Data for Pharmaceutical Formulation (0.2 mg capsules;
30° C./65% RH storage)

| | Test | |
|---|---|---|
| | T = 0 | T = 6 |
| Appearance | Conforms | Conforms |
| Deliverable mass | 100% | 100% |
| % Gal d 1 | 13% | 15% |
| % Gal d 2 | 78% | 77% |
| % Gal d 3 | 4% | 4% |
| % Gal d 4 | 0.5% | 0.5% |
| Relative potency | 0.8 | 1.2 |
| Total protein (BCA) | 91% | 91% |

The major allergens and the relative potency of the pharmaceutical formulation was stable between 0 and 6 months after accelerated stability conditions of 30° C./65% RH storage.

The results of the 0.2 mg capsule after 40° C./75% RH stressed stability storage condition are summarized in the table, below. For the 6-month time point, both the older RP-HPLC method and the improved RP-HPLC method described in the preceding example were employed, but only the older RP-HPLC method values are reported for comparison the T=0, 1, and 3 month time points.

TABLE 21
Stability Data for Pharmaceutical Formulation (0.2 mg capsules;
40° C./75% RH storage)

| | Test | | | |
|---|---|---|---|---|
| | T = 0 | T = 1 | T = 3 | T = 6 |
| Appearance | Conforms | Conforms | Conforms | Conforms |
| Deliverable | 100% | 100% | 99% | 100% |
| mass | | | | |
| % Gal d 1 | 13% | 16% | 15% | 14% |
| % Gal d 2 | 78% | 73% | 76% | 78% |
| % Gal d 3 | 4% | 4% | 4% | 4% |
| % Gal d 4 | 0.5% | 0.3% | 0.3% | 0.5% |
| Relative potency | 0.8 | 1.3 | 1.3 | 1.3 |
| Total protein | 91% | 73% | 75% | 80% |
| (BCA) | | | | |

The major allergens and the relative potency of the pharmaceutical formulation was stable between 0 and 6 months, even under the stressed stability storage condition of 40° C./75% RH.

The stability study was completed with lots of capsules comprising doses of 1 mg, 6 mg, 12 mg, and 300 mg drug product. The results demonstrated that the pharmaceutical formulations are stable under different storage conditions across dosage levels.

### Example 8: Oral Immunotherapy of a Patient for Egg Allergy

A patient is treated for a diagnosed egg allergy with a pharmaceutical composition of egg white protein. The patient undergoes an initial dose escalation, an up-dosing phase, and a maintenance phase according to an oral immunotherapy schedule. An indicated dose during either the up-dosing phase or the maintenance phase may be adjusted if the subject experiences an adverse allergic reaction to a previous dose and/or experiences a concurrent factor associated with increased sensitivity to an allergen that is not related to the administration of a dose of the pharmaceutical formulation.

The patient's serum total IgE, egg-white-specific IgE, total IgG4, and egg-white-specific IgG4 levels may be assessed before the first dose of the pharmaceutical composition, during the oral immunotherapy, and/or after the oral immunotherapy. The patient's reactivity to doses of raw egg white and/or baked egg white may be assessed before, during, and/or after the oral immunotherapy.

### Example 9: Low-Dose Manufacturing of an Egg White Protein Formulation

A formulated pharmaceutical composition used for the manufacture of low doses, including 0.2 mg and 1 mg doses, for oral immunotherapy can be made as follows. The starting drug substance is dried egg white protein powder. The dried egg white protein powder is characterized to ensure consistent total protein levels and allergen levels, such as levels of Gal d 1, Gal d 2, Gal d 3, and Gal d 4, by comparison to a reference standard or predefined acceptance criteria. The relative potency of the egg white protein powder is also confirmed, such as by an ELISA against ovomucoid, and compared to a predefined acceptance criteria.

The dried egg white protein powder is manually mixed with a first portion of pregelatinized starch to form a first mixture. The first mixture is then co-sieved through a mesh screen with a second portion of pregelatinized starch, and the co-sieved material is then manually mixed to form a second mixture. The second mixture is then mixed with a third portion of pregelatinized starch by a blender (e.g., a tumble blender) to form a third mixture. The third mixture is then mixed with a fourth portion of pregelatinized starch by a blender (e.g., a tumble blender) to form a fourth mixture. The fourth mixture is then mixed with a fifth portion of pregelatinized starch by a blender (e.g., a tumble blender) to form a fifth mixture. The fifth mixture is then mixed with microcrystalline cellulose by a blender (e.g., a tumble blender), and then further mixed by high-shear mixing (e.g., in a conical mill) to form a sixth mixture. Separately, a sixth portion of pregelatinized starch and magnesium stearate are mixed together to form a seventh mixture, which is then passed through a mesh screen to sieve the seventh mixture. The seventh mixture is then mixed in a blender (e.g., a tumble blender) with the sixth mixture to form the egg white protein formulation.

Once formulated, the bulk formulation is characterized by assessment of total protein, content uniformity, individual allergen levels (such as levels of Gal d 1, Gal d 2, Gal d 3, and Gal d 4), the relative potency of the allergens, such as by ELISA against ovomucoid, and the water activity. Each assessment is judged against predetermined acceptance criteria. Once the bulk formulation is validated, it is distributed into containers, such as capsules or sachets. Containers from the same manufacturing batch form a lot. Individual containers, or multiple containers from the same lot, are also assessed. Validated lots comprising a plurality of containers are then distributed for use in oral immunotherapy of egg allergy.

### Example 10: Low-Dose Manufacturing of an Egg White Protein Formulation

A formulated pharmaceutical composition used for the manufacture of low doses, including 3 mg and 6 mg doses, for oral immunotherapy can be made as follows. The starting drug substance is dried egg white protein powder. The dried egg white protein powder is characterized to ensure consistent total protein levels and allergen levels, such as levels of Gal d 1, Gal d 2, Gal d 3, and Gal d 4, by comparison to a reference standard or predefined acceptance criteria. The relative potency of the egg white protein powder is also confirmed, such as by an ELISA against ovomucoid, and compared to a predefined acceptance criteria.

The dried egg white protein powder is mixed with a first portion of pregelatinized starch to form a first mixture. The first mixture is mixed with a second portion of pregelatinized starch by high-shear mixing (for example, a conical mill), and then further mixed in a blender (such as a tumble blender) to form a second mixture. The second mixture is then mixed with a third portion of pregelatinized starch to form a third mixture. The third mixture is then mixed with microcrystalline cellulose in a blender (such as a tumble blender) to form a fourth mixture, which is then further mixed by high-shear mixing (e.g., in a conical mill). Separately, a fourth portion of pregelatinized starch and magnesium stearate are mixed together to form a fifth mixture, which is passed through a mesh screen. The sieved fifth mixture is then mixed with the fourth mixture to form the egg white protein formulation.

Once formulated, the bulk formulation is characterized by assessment of total protein, content uniformity, individual allergen levels (such as levels of Gal d 1, Gal d 2, Gal d 3, and Gal d 4), the relative potency of the allergens, such as by ELISA against ovomucoid, and the water activity. Each assessment is judged against predetermined acceptance criteria. Once the bulk formulation is validated, it is distributed into containers, such as capsules or sachets. Containers from the same manufacturing batch form a lot. Individual containers, or multiple containers from the same lot, are also assessed. Validated lots comprising a plurality of containers are then distributed for use in oral immunotherapy of egg allergy.

### Example 11: Medium-Dose Manufacturing of an Egg White Protein Formulation

A formulated pharmaceutical composition used for the manufacture of medium doses, including 12 mg doses, for oral immunotherapy can be made as follows. The starting drug substance is dried egg white protein powder. The dried egg white protein powder is characterized to ensure consistent total protein levels and allergen levels, such as levels of Gal d 1, Gal d 2, Gal d 3, and Gal d 4, by comparison to a reference standard or predefined acceptance criteria. The relative potency of the egg white protein powder is also confirmed, such as by an ELISA against ovomucoid, and compared to a predefined acceptance criteria.

The dried egg white protein powder is manually mixed with a first portion of pregelatinized starch and colloidal silicon dioxide to form a first mixture. The first mixture is then mixed with a second portion of pregelatinized starch using high-shear mixing (for example, in a conical mill) to form a second mixture, and the second mixture is then further mixed in a blender (e.g., a tumble blender) at a lower shear force. The second mixture is then mixed (using, for example, a blender, such as a tumble blender) with a third portion of pregelatinized starch to form a third mixture. The third mixture is then mixed with a fourth portion of pregelatinized starch and microcrystalline cellulose in a blender (such as a tumble blender) to form a fourth mixture, which is further mixed by high-shear mixing (for example, using a conical mill). Separately, a fifth portion of pregelatinized starch is mixed with magnesium stearate to form a fifth mixture, which is passed through a mesh screen. The sieved fifth mixture is then mixed with the fourth mixture to form the egg white protein formulation.

Once formulated, the bulk formulation is characterized by assessment of total protein, content uniformity, individual allergen levels (such as levels of Gal d 1, Gal d 2, Gal d 3, and Gal d 4), the relative potency of the allergens, such as by ELISA against ovomucoid, and the water activity. Each assessment is judged against predetermined acceptance criteria. Once the bulk formulation is validated, it is distributed into containers, such as capsules or sachets. Containers from the same manufacturing batch form a lot. Individual containers, or multiple containers from the same lot, are also assessed. Validated lots comprising a plurality of containers are then distributed for use in oral immunotherapy of egg allergy.

### Example 12: Medium-Dose Manufacturing of an Egg Protein Formulation

A formulated pharmaceutical composition used for the manufacture of medium doses, including 20 mg and 40 mg doses, for oral immunotherapy can be made as follows. The starting drug substance is dried egg white protein powder. The dried egg white protein powder is characterized to ensure consistent total protein levels and allergen levels, such as levels of Gal d 1, Gal d 2, Gal d 3, and Gal d 4, by comparison to a reference standard or predefined acceptance criteria. The relative potency of the egg white protein powder is also confirmed, such as by an ELISA against ovomucoid, and compared to a predefined acceptance criteria.

The dried egg white protein powder is manually mixed with a first portion of pregelatinized starch and colloidal silicon dioxide to form a first mixture. The first mixture is then mixed with a second portion of pregelatinized starch by high-shear mixing (e.g., in a conical mill) to form a second mixture, which is then further mixed in a blender (e.g., a tumble blender). The second mixture is then mixed with a third portion of pregelatinized starch and microcrystalline cellulose in a blender (such as a tumble blender) to form a third mixture, which is then further mixed by high-shear mixing (for example, in a conical mill). Separately, a fourth portion of pregelatinized starch and magnesium stearate are mixed to form a fourth mixture, which is then passed through a mesh screen. The sieved fourth mixture is then mixed with the third mixture to form the egg white protein formulation.

Once formulated, the bulk formulation is characterized by assessment of total protein, content uniformity, individual allergen levels (such as levels of Gal d 1, Gal d 2, Gal d 3, and Gal d 4), the relative potency of the allergens, such as by ELISA against ovomucoid, and the water activity. Each assessment is judged against predetermined acceptance criteria. Once the bulk formulation is validated, it is distributed into containers, such as capsules or sachets. Containers from the same manufacturing batch form a lot. Individual containers, or multiple containers from the same lot, are also assessed. Validated lots comprising a plurality of containers are then distributed for use in oral immunotherapy of egg allergy.

### Example 13: High-Dose Manufacturing of an Egg White Protein Formulation

A formulated pharmaceutical composition used for the manufacture of high doses, including 120 mg, 160 mg, 200 mg, 240 mg, and 300 mg doses, for oral immunotherapy can be made as follows. The dried egg white protein powder is characterized to ensure consistent total protein levels and allergen levels, such as levels of Gal d 1, Gal d 2, Gal d 3, and Gal d 4, by comparison to a reference standard or predefined acceptance criteria. The relative potency of the egg white protein powder is also confirmed, such as by an ELISA against ovomucoid, and compared to a predefined acceptance criteria.

The dried egg white protein powder is manually mixed with a first portion of pregelatinized starch and colloidal silicon dioxide to form a first mixture. The first mixture is then mixed with microcrystalline cellulose by high-shear mixing (for example, in a conical mill) to form a second mixture, which is then further mixed in a blender (such as a tumble blender). Separately, a second portion of pregelatinized starch is mixed with magnesium stearate to form a third mixture, which is then passed through a mesh screen. The sieved third mixture is then mixed with the second mixture in a blender (such as a tumble blender) to form the egg white protein formulation.

Once formulated, the bulk formulation is characterized by assessment of total protein, content uniformity, individual allergen levels (such as levels of Gal d 1, Gal d 2, Gal d 3, and Gal d 4), the relative potency of the allergens, such as by ELISA against ovomucoid, and the water activity. Each assessment is judged against predetermined acceptance criteria. Once the bulk formulation is validated, it is distributed into containers, such as capsules or sachets. Containers from the same manufacturing batch form a lot. Individual containers, or multiple containers from the same lot, are also assessed. Validated lots comprising a plurality of containers are then distributed for use in oral immunotherapy of egg allergy.

What is claimed is:

1. A method of making an egg white protein formulation, comprising:
(a) mixing dried egg white protein powder with a first amount of a first diluent to form a first mixture;
(b) passing the first mixture through a mesh screen;
(c) mixing the first mixture with a second amount of the first diluent to form a second mixture after steps (a) and (b);
(d) mixing the second mixture with a second diluent to form a third mixture;
(e) mixing the third mixture at a higher shear force than used for mixing in step (c); and
(f) mixing the third mixture with a lubricant to form the egg white protein formulation.

2. The method of claim 1, wherein the egg white protein formulation has about 0.05 wt % to about 2.5 wt % of egg white protein.

3. The method of claim 1 or 2, wherein the egg white protein formulation has about 0.1 wt % to about 0.7 wt % egg white protein.

4. The method of any one of claims 1-3, wherein step (b) comprises passing at least a portion of the second amount of the first diluent through the mesh screen with the first mixture.

5. The method of any one of claims 1-3, wherein step (b) comprises passing the second amount of the first diluent through the mesh screen with the first mixture.

6. The method of any one of claims 1-5, wherein step (c) comprises a plurality of sub-steps, wherein each sub-step comprises (i) adding a portion of the second amount of the first diluent to the first mixture, and (ii) mixing the portion of the second amount of the first diluent and the first mixture.

7. The method of claim 6, wherein step (c) comprises three or more sub-steps.

8. The method of any one of claims 1-5, wherein step (c) comprises continuously mixing the first mixture and the second amount of the first diluent as the second amount of the first diluent is added to the first mixture.

9. The method of any one of claims 1-8, wherein the first mixture is mixed with the second amount of the first diluent in a tumble blender.

10. The method of any one of claims 1-9, wherein the second mixture is mixed with the second diluent in a tumble blender.

11. The method of any one of claims 1-10, wherein the third mixture is mixed using a conical mill.

12. The method of any one of claims 1-11, wherein the third mixture is mixed with the lubricant in a tumble blender.

13. The method of any one of claims 1-12, wherein a third amount of the first diluent is mixed with the third mixture.

14. The method of claim 13, wherein the third amount of the first diluent and the lubricant are co-mixed with the third mixture.

15. The method of any one of claims 1-14, comprising mixing the lubricant with an additional portion of the first diluent or the second diluent before mixing the lubricant with the third mixture.

16. The method of claim 15, wherein the mixture of the lubricant and the additional portion of the first diluent or the second diluent is passed through a mesh screen before the lubricant is mixed with the third mixture.

17. A method of making an egg white protein formulation, comprising:
(a) mixing dried egg white protein powder with a first amount of a first diluent to form a first mixture;
(b) co-sieving the first mixture with a second portion of the first diluent through a mesh screen to form a second mixture;
(c) serially diluting the second mixture using one or more additional portions of the first diluent to form a third mixture;
(d) mixing the third mixture with a second diluent to form a fourth mixture;
(e) mixing an additional portion of the first diluent or the second diluent with a lubricant to form a fifth mixture; and
(f) mixing the fourth mixture with the fifth mixture.

18. The method of claim 17, comprising mixing the second mixture prior to step (c).

19. The method of claim 17 or 18, comprising, prior to step (e), mixing the fourth mixture using a higher shear force than used to mix the third mixture with the second diluent.

20. The method of any one of claims 17-19, comprising, prior to step (f), sieving the fifth mixture.

21. The method of any one of claims 1-20, wherein the egg white protein formulation is substantially free of colloidal silicon dioxide.

22. The method of any one of claims 1-21, wherein the egg white protein formulation is about 40 wt % to about 70 wt % of the first diluent.

23. The method of any one of claims 1-22, wherein the egg white protein formulation is about 30 wt % to about 50 wt % of the second diluent.

24. The method of any one of claims 1-23, wherein the egg white protein formulation is about 0.1 wt % to about 2 wt % of the lubricant.

25. A method of making an egg white protein formulation, comprising:
(a) mixing dried egg white protein powder with a first amount of a first diluent to form a first mixture;
(b) mixing a second amount of the first diluent with the first mixture to form a second mixture;
(c) mixing the second mixture with a second diluent to form a third mixture;
(d) mixing the third mixture at a higher shear force than used for mixing in step (b); and
(e) mixing the third mixture with a lubricant to form the egg white protein formulation.

26. The method of claim 25, wherein the dried egg white protein powder is mixed with the first amount of the first diluent in step (a) at a higher shear force than used for mixing in step (b).

27. The method of claim 25 or 26, wherein the egg white protein formulation has about 1 wt % to about 70 wt % egg white protein.

28. The method of any one of claims 25-27, the dried egg white protein powder is mixed with the first amount of the first diluent in step (a) using a conical mill.

29. The method of any one of claims 25-28, wherein step (b) comprises two mixing sub-steps, wherein one mixing sub-step is at a higher shear force than the other mixing sub-step.

30. The method of any one of claims 25-29, wherein step (c) further comprises mixing the second mixture with an additional amount of the first diluent prior to mixing with the second diluent to form the third mixture.

31. The method of any one of claims 25-29, wherein step (c) further comprises co-mixing the second mixture with an additional amount of the first diluent and the second diluent to form the third mixture.

32. The method of any one of claims 25-31, the third mixture is mixed in step (d) using a conical mill.

33. The method of any one of claims 25-32, wherein the second amount of the first diluent and the first mixture are mixed in a tumble blender.

34. The method of any one of claims 25-33, wherein the second mixture is mixed with the second diluent in a tumble blender.

35. The method of any one of claims 25-33, wherein a third amount of the first diluent is mixed with the third mixture.

36. The method of claim 35, wherein the third amount of the first diluent and the lubricant are co-mixed with the third mixture.

37. The method of any one of claims 25-36, wherein step (e) comprises: (i) mixing a portion of the third mixture with a lubricant; (ii) passing the mixture of (i) through a mesh screen; and (iii) mixing the mixture of (ii) with an additional portion of the third mixture to form the egg white protein formulation.

38. The method of any one of claims 25-37, wherein the third mixture is mixed with the lubricant in a tumble blender.

39. The method of any one of claims 25-38, wherein the egg white protein formulation is about 9 wt % to about 85 wt % of the first diluent.

40. The method of any one of claims 25-39, wherein the egg white protein formulation is about 10 wt % to about 50 wt % of the second diluent.

41. The method of any one of claims 25-40, wherein the egg white protein formulation is about 10 wt % to about 20 wt % of the second diluent.

42. The method of any one of claims 25-41, wherein the egg white protein formulation is about 0.1 wt % to about 2 wt % of the lubricant.

43. The method of any one of claims 25-42, wherein the egg white protein formulation comprises a glidant.

44. The method of claim 43, wherein the glidant is mixed with the egg white protein powder and the first amount of the first diluent during or prior to step (a).

45. The method of claim 43 or 44, wherein the glidant is colloidal silicon dioxide.

46. The method of any one of claims 25-45, wherein the egg white protein formulation is about 2 wt % to about 70 wt % egg white protein.

47. The method of any one of claims 25-44, wherein the egg white protein formulation is substantially free of colloidal silicon dioxide.

48. The method of claim 47, wherein the egg white protein formulation is about 1 wt % to about 5 wt % egg white protein.

49. A method of making an egg white protein formulation, comprising:
(a) mixing dried egg white protein powder, a first diluent, and a glidant to form a first mixture;
(b) mixing a second diluent and the first mixture at a higher shear force than used for mixing in step (a) to form a second mixture;
(c) mixing the second mixture and a lubricant to form the egg white protein formulation.

50. The method of claim 49, wherein the egg white protein formulation has about 50 wt % to about 80 wt % of egg white protein.

51. The method of claim 49 or 50, wherein the dried egg white protein powder, the first diluent, and the glidant are mixed in a tumble blender.

52. The method of any one of claims 49-51, wherein the second diluent and the first mixture are mixed in step (b) using a conical mill.

53. The method of any one of claims 49-52, wherein the second mixture and the lubricant are mixed in a tumble blender.

54. The method of any one of claims 49-53, wherein a second amount of the first diluent is mixed with the second mixture.

55. The method of claim 54, wherein the second amount of the first diluent and the lubricant are co-mixed with the second mixture.

56. The method of any one of claims 49-55, wherein the glidant comprises colloidal silicon dioxide.

57. The method of any one of claims 49-56, wherein step (c) comprises: (i) mixing a portion of the second mixture and the lubricant; (ii) passing the portion of the second mixture and the lubricant through a mesh screen; and (iii) mixing (ii) with an additional portion of the second mixture to form the egg white protein formulation.

58. The method of any one of claims 1-57, wherein the egg white protein formulation is made in a lot size of about 5 kg or more.

59. The method of claim 58, wherein the egg white protein formulation is made in a lot size of about 5 kg to about 50 kg.

60. The method of any one of claims 1-59, comprising determining an egg white protein blend uniformity for the egg white protein formulation.

61. The method of any one of claims 1-60, wherein the egg white protein formulation has an egg white protein blend uniformity relative standard deviation (RSD) of about 15% or less.

62. The method of any one of claims 1-61, comprising packaging the egg white protein formulation in a plurality of dosage containers.

63. The method of claim 62, wherein the dosage containers are capsules or sachets.

64. The method of claim 62 or 63, comprising determining an egg white protein content uniformity for the plurality of dosage containers.

65. The method of any one of claims 62-64, wherein the plurality of dosage containers has an egg white protein content uniformity relative standard deviation (RDS) of about 15% or less.

66. The method of any one of claims 1-65, wherein the first diluent is pregelatinized starch.

67. The method of any one of claims 1-66, wherein the second diluent is microcrystalline cellulose.

68. The method of any one of claims 1-67, wherein the lubricant is magnesium stearate.

69. The method of any one of claims 1-68, wherein the dried egg white protein powder comprises about 50 wt % to about 90 wt % egg white protein.

70. The method of any one of claims 1-69, wherein formation of the dried egg white protein powder comprises spray drying liquid egg whites.

71. The method of any one of claims 1-70, wherein the egg white protein powder has had glucose removed.

72. The method of any one of claims 1-71, wherein the dried egg white powder has been pasteurized.

73. The method of any one of claims 1-72, wherein the dried egg white protein powder is derived from a chicken egg.

74. The method of anyone of claims 1-73, further comprising characterizing ovomucoid, ovalbumin, ovotransferrin, or lysozyme in the dried egg white protein powder.

75. The method of anyone of claims 1-74, further comprising characterizing ovomucoid, ovalbumin, ovotransferrin, or lysozyme in the egg white protein formulation.

76. The method of claim 74 or 75, wherein characterizing ovomucoid, ovalbumin, ovotransferrin, or lysozyme comprises obtaining a high-performance liquid chromatography (HPLC) profile.

77. The method of claim 76, wherein the HPLC profile is a revere-phased HPLC (RP-HPLC) profile.

78. The method of claim 76, wherein the HPLC profile is a size-exclusion chromatography HPLC (SEC-HPLC) profile.

79. The method of any one of claims 76-78, comprising comparing the obtained HPLC profile to a reference HPLC profile.

80. The method of any one of claims 74-79, wherein characterizing ovomucoid, ovalbumin, ovotransferrin, or lysozyme comprises quantifying an amount of ovomucoid, ovalbumin, ovotransferrin, or lysozyme.

81. The method of claim 80, wherein quantifying the amount of ovomucoid, ovalbumin, ovotransferrin, or lysozyme comprises measuring an amount of ovomucoid, ovalbumin, ovotransferrin, or lysozyme compared to total protein in the egg white protein powder or the egg white protein formulation.

82. The method of claim 80, wherein quantifying the amount of ovomucoid, ovalbumin, ovotransferrin, or lysozyme comprises measuring an amount of ovomucoid, ovalbumin, ovotransferrin, or lysozyme compared to a total amount of ovomucoid, ovalbumin, ovotransferrin, and lysozyme in the egg white protein powder or the egg white protein formulation.

83. The method of any one of claims 74-82, wherein characterizing ovomucoid, ovalbumin, ovotransferrin, or lysozyme comprises measuring a potency of ovomucoid, ovalbumin, ovotransferrin, or lysozyme in the egg white protein powder or the egg white protein formulation.

84. The method of claim 83, wherein the potency of ovomucoid, ovalbumin, ovotransferrin, or lysozyme in the egg white protein powder or the egg white protein formulation is measured relative to a potency of ovomucoid, ovalbumin, ovotransferrin, or lysozyme in a reference sample.

85. The method of claim 83 or 84, wherein the potency of ovomucoid, ovalbumin, ovotransferrin, or lysozyme is measured using an immunoassay.

86. The method of claim 85, wherein the immunoassay comprises the use of one or more of an antibody that specifically binds ovomucoid, an antibody that specifically binds ovalbumin, an antibody that specifically binds ovotransferrin, or an antibody that specifically binds lysozyme.

87. The method of claim 85, wherein the immunoassay comprises the use of a pool of antibodies comprising two or more antibodies selected from the group consisting of an antibody that specifically binds ovomucoid, an antibody that specifically binds ovalbumin, an antibody that specifically binds ovotransferrin, and an antibody that specifically binds lysozyme.

88. The method of claim 86 and 87, wherein the antibody is an IgE antibody or an IgG antibody.

89. The method of any one of claims 84-88, wherein the potency of ovomucoid, ovalbumin, ovotransferrin, or lysozyme is measured using an enzyme-linked immunosorbent assay (ELISA).

90. An egg white protein formulation made according to the method of any one of claims 1-89.

91. An egg white protein formulation, comprising dried egg white protein powder, a first diluent, a second diluent, and a lubricant, wherein the egg white protein formulation is substantially free of colloidal silicon dioxide.

92. The egg white protein formulation of claim 91, wherein the egg white protein formulation comprises about 0.1 wt % to about 3.5 wt % egg white protein.

93. The egg white protein formulation of claim 91 or 92, wherein the first diluent is pregelatinized starch.

94. The egg white protein formulation of any one of claims 91-93, wherein the second diluent is microcrystalline cellulose.

95. The egg white protein formulation of any one of claims 91-94, wherein the lubricant is magnesium stearate.

96. The egg white protein formulation of any one of claims 91-95, wherein the egg white protein formulation consists essentially of the egg white protein powder, the first diluent, the second diluent, and the lubricant.

97. A method of treating an egg allergy in a patient, comprising:
orally administering to the patient a plurality of doses of a pharmaceutical composition comprising egg white protein according to an oral immunotherapy schedule comprising:
(a) an up-dosing phase comprising orally administering to the patient a series of escalating doses of about 1 mg to about 300 mg of egg white protein, wherein a given dose is administered to the patient for at least two weeks before the dose is escalated, and wherein the up-dosing phase is about 20 weeks to about 44 weeks in length; and
(b) a maintenance phase comprising orally administering to the patient a plurality of maintenance doses comprising egg white protein, wherein the maintenance phase is about 12 weeks in length or more.

98. The method of claim 97, wherein the patient has an egg-white-specific serum IgE (ew-IgE) level of about 7 kU_{A}/L or more at the start of treatment.

99. The method of claim 97, wherein the patient has an egg-white-specific serum IgE (ew-IgE) level of about 5 kU_{A}/L or more at the start of treatment.

100. The method of any one of claims 97-99, wherein the series of escalating doses administered to the patient during the up-dosing phase comprises at least a 1 mg dose of egg white protein and a 300 mg dose of egg white protein.

101. The method of any one of claims 97-100, wherein the series of escalating doses administered to the patient during the up-dosing phase comprises at least 10 different doses of egg white protein.

102. The method of any one of claims 97-101, wherein the series of escalating doses administered to the patient during the up-dosing phase comprises doses of about 1 mg, about 3 mg, about 6 mg, about 12 mg, about 20 mg, about 40 mg, about 80 mg, about 120 mg, about 160 mg, about 200 mg, about 240 mg, and about 300 mg of egg white protein.

103. The method of any one of claims 97-102, wherein a dose administered during the up-dosing phase is escalated only if the patient tolerates the previous dose.

104. The method of any one of claims 97-103, wherein the maximum dose administered to the patient during the up-dosing phase is about 300 mg of egg white protein.

105. The method of any one of claims 97-104, wherein the maintenance dose administered to the patient during the maintenance phase is about 300 mg of egg white protein or more.

106. The method of any one of claims 97-105, wherein the maintenance dose administered to the patient during the maintenance phase is about 300 mg of egg white protein.

107. The method of any one of claims 97-106, wherein the maintenance dose is administered to the patient only if the patient tolerates the maximum dose administered to the patient during the up-dosing phase.

108. The method of any one of claims 97-107, wherein the patient tolerates a dose of about 600 mg raw egg white protein at the end of the maintenance phase.

109. The method of any one of claims 97-108, wherein the patient tolerates a dose of about 1000 mg raw egg white protein at the end of the maintenance phase.

110. The method of any one of claims 97-109, wherein the patient tolerates a dose of about 2000 mg raw egg white protein at the end of the maintenance phase.

111. The method of any one of claims 97-110, wherein the patient tolerates a cumulative dose of about 2000 mg cooked egg white protein at the end of the maintenance phase.

112. The method of any one of claims 97-111, wherein the patient tolerates a cumulative dose of about 2000 mg baked egg white protein at the end of the maintenance phase.

113. The method of any one of claims 97-112, wherein the patient is unable to tolerate a dose of about 300 mg of raw egg white protein prior to the start of treatment.

114. The method of any one of claims 97-113 wherein the patient is unable to tolerate a cumulative dose of about 2000 mg of cooked egg white protein prior to the start of treatment.

115. The method of any one of claims 97-114, wherein the patient is unable to tolerate a cumulative dose of about 2000 mg of baked egg white protein prior to the start of treatment.

116. The method of any one of claims 97-113, wherein the patient tolerates a cumulative dose of about 2000 mg of cooked egg white protein prior to the start of treatment.

117. The method of any one of claims 97-113 and 116, wherein the patient tolerates a cumulative dose of about 2000 mg of baked egg white protein prior to the start of treatment.

118. The method of any one of claims 97-117, wherein the oral immunotherapy schedule comprises an initial escalation phase prior to the up-dosing phase, the initial escalation phase comprising orally administering to the patient a series of escalating doses of about 0.2 mg to about 2 mg of egg white protein in a single day, wherein a single administration of any given dose is administered to the patient, and wherein the doses are spaced by at least 15 minutes.

119. The method of claim 118, wherein the patient is treated according to the oral immunotherapy schedule only if the patient tolerates a dose of about 1.0 mg of raw egg white protein on the first day of treatment.

120. The method of any one of claims 97-119, wherein the patient is about 4 years of age or older prior to the start of treatment.

121. The method of any one of claims 97-120, wherein the patient is about 4 years to about 26 years of age prior to the start of treatment.

122. A method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising:
orally administering to the patient a first dose of the pharmaceutical composition; and
orally administering to the patient a second dose of the pharmaceutical composition, wherein the second dose is reduced, skipped, or at least a portion of the dose is delayed if the patient experiences an adverse event related to the administration of the first dose.

123. The method of claim 122, wherein the second dose is divided into a first portion and a second portion, wherein the first portion is administered according to a predetermined dosing schedule, and wherein the second portion is delayed relative to the predetermined dosing schedule, if the patient experiences the adverse event related to the administration of the first dose.

124. The method of claim 123, wherein the second portion is delayed by about 8 hours to about 12 hours after the first portion is administered.

125. The method of claim 122, wherein the second dose is skipped if the patient experiences the adverse event related to the administration of the first dose.

126. The method of claim 122, wherein the second dose is reduced relative to the first dose if the patient experiences the adverse event related to the administration of the first dose.

127. The method of claim 126, wherein subsequent doses of the pharmaceutical composition are reduced relative to the first dose for about one week or more prior to escalating the subsequent doses.

128. The method of claim 126 or 127, wherein subsequent doses of the pharmaceutical composition are reduced relative to the first dose for about one week to about two weeks prior to attempting to escalate the subsequent doses.

129. The method of any one of claims 126-128, wherein subsequent doses of the pharmaceutical composition are reduced relative to the first dose for about one week to about two weeks prior to escalating the subsequent doses.

130. The method of any one of claims 122-129, wherein the adverse event related to the administration of the first dose is a mild allergenic adverse event.

131. The method of any one of claims 122-130, wherein the adverse event related to the administration of the first dose is a moderate allergenic adverse event or a severe allergenic adverse event.

132. The method of any one of claims 122-131, wherein the first dose and the second dose are administered to the patient during the up-dosing phase of the oral immunotherapy.

133. The method of any one of claims 122-131, wherein the first dose and the second dose are administered to the patient during the maintenance phase of the oral immunotherapy.

134. A method of adjusting a dosage of a pharmaceutical composition comprising egg white protein during oral immunotherapy for an egg allergy in a subject, the oral immunotherapy comprising (i) an up-dosing phase comprising orally administering to the patient a series of escalating doses of the egg white protein, and (ii) a maintenance phase comprising orally administering to the patient a plurality of maintenance doses comprising the egg white protein; the method comprising:
orally administering to the patient a first dose of the pharmaceutical composition; and
orally administering to the patient a second dose of the pharmaceutical composition, wherein the second dose is reduced or skipped if the patient experiences a concurrent factor associated with increased sensitivity to an allergen that is not related to the administration of the first dose.

135. The method of claim 134, wherein the concurrent factor associated with increased sensitivity to an allergen is an atopic disease flare-up, inflammation, an illness, or menses.

136. The method of claim 134 or 135, wherein the second dose is skipped if the patient experiences the concurrent factor associated with increased sensitivity to an allergen not related to the administration of the first dose.

137. The method of claim 134 or 135, wherein the second dose is reduced relative to the first dose if the patient experiences the concurrent factor associated with increased sensitivity to an allergen not related to the administration of the first dose.

138. The method of claim 137, wherein subsequent doses of the pharmaceutical composition are reduced relative to the first dose for about one week or more prior to escalating the subsequent doses.

139. The method of claim 137 or 138, wherein subsequent doses of the pharmaceutical composition are reduced relative to the first dose for about one week to about two weeks prior to attempting to escalate the subsequent doses.

140. The method of any one of claims 137-139, wherein subsequent doses of the pharmaceutical composition are reduced relative to the first dose for about one week to about two weeks prior to escalating the subsequent doses.

141. The method of any one of claims 134-140, wherein the first dose and the second dose are administered to the patient during the up-dosing phase of the oral immunotherapy.

142. The method of any one of claims 134-141, wherein the first dose and the second dose are administered to the patient during the maintenance phase of the oral immunotherapy.

143. The method of any one of claims 134-142, wherein the concurrent factor associated with increased sensitivity to an allergen is an unintended exposure to a food that the patient is allergic to.

144. The method of any one of claims 97-143, wherein the egg white protein in the pharmaceutical composition is raw egg white protein.

145. The method of any one of claims 97-144, wherein the pharmaceutical composition is mixed with a food vehicle prior to administration.

146. A pharmaceutical composition for use in the method of any one of claims 97-145.

147. The pharmaceutical composition of claim 146, wherein the pharmaceutical composition comprises the egg white protein formulation prepared according to the method of any one of claims 1-96.

148. A pharmaceutical composition for use in the manufacture of a medicament for a method of treating an egg allergy in a patient according to the method of any one of claims 97-145.

149. The pharmaceutical composition of claim 148, wherein the pharmaceutical composition comprises the egg white protein formulation prepared according to the method of any one of claims 1-96.
